(19) United States
(12) Patent Application Publication
(10) Pub. No.: US 2024/0317826 A1
(43) Pub. Date: Sep. 26, 2024

(54) COMPOSITIONS AND METHODS FOR TREATING AND/OR IDENTIFYING AN AGENT FOR TREATING HIV-1 INFECTIONS

(71) Applicants: Albert Einstein College of Medicine, Bronx, NY (US); New York Blood Center, Inc., New York, NY (US)

(72) Inventors: Ganjam V. Kalpana, Scarsdale, NY (US); Asim Kumar Debnath, Fort Lee, NJ (US)

(21) Appl. No.: 18/290,059

(22) PCT Filed: May 11, 2022
(86) PCT No.: PCT/US22/28726

§ 371 Date: Nov. 9, 2023

Related U.S. Application Data
(60) Provisional application No. 63/187,270, filed on May 11, 2021.

(51) Int. Cl. C07K 14/47; A61K 38/00; A61K 45/06; A61P 31/18

(52) U.S. Cl. CPC C07K 14/4702; A61K 45/06; A61P 31/18; A61K 38/00; C07K 2319/21; C07K 2319/23

(57) ABSTRACT

The present invention relates, in part, to compositions and methods for treating and/or identifying an agent for treating HIV infections.

### CROSS-REFERENCE TO RELATED APPLICATIONS

This application claims the benefit of U.S. Provisional Application No. 63/187,270, filed on May 11, 2021, the entire contents of which are incorporated herein in their entirety by this reference.

### STATEMENT OF GOVERNMENTAL RIGHTS

This invention was made with government support under GM112520-04S1 and R21AI156932-01 awarded by the National Institutes of Health. The government has certain rights in the invention.

### BACKGROUND OF THE INVENTION

The AIDS pandemic continues to affect ˜38,000,000 people worldwide. Despite the advent of multiple combinations of antiretroviral therapies (cART), HIV has not been eradicated, and currently there are no vaccines. While the current cART drugs inhibit circulating plasma viral replication, they are not effective in eliminating latent reservoirs. During replication, the HIV-1 viral DNA becomes integrated into host genome to become a “provirus”, which is an essential step in viral replication. This integrated provirus is subsequently transcribed to produce viral RNA, which is translated and the viral proteins are assembled to produce progeny virions. In the infected patients undergoing antiretroviral therapy, integrated HIV provirus often becomes transcriptionally silent and constitutes a pool of persistent or latent reservoir. These latent reservoirs cannot be detected by the immune system as they do not produce viral RNA and/or proteins and hence cannot be eliminated. The HIV reservoirs thus pose a major obstacle to eradicate HIV-1. When these latent reservoirs gets reactivated, they become the source for rebound virus in patients and often these viruses accumulate resistant mutations to current cART and hence are not easily treatable, presenting a major obstacle to suppress rebound virus. Thus two major problems associated with current HIV-1 treatment strategy are: (i) inability to eliminate latent reservoirs; and (ii) drug resistant viruses.

The HIV cure/eradication strategies are at the forefront of NIH priorities for HIV-1 research. Many strategies have been proposed to eradicate HIV-1 from ART-suppressed patients. One such strategy is called “shock and kill” therapy, where the latent cells are activated with Latency Reversing Agents (LRA) that reactivate the transcriptionally silent provirus, the infected cells are subsequently eliminated by immune system and viral spread is suppressed by simultaneously treating with antiretrovirals.

Currently, there are several FDA approved anti-HIV drugs that target entry, reverse transcription, integration and proteolytic processing, which are often used in combination1. A majority of these drugs target viral enzymes (reverse transcriptase, integrase and protease). Because of the high mutation rates, the virus develops resistance to these drugs and hence a cocktail of drugs are used to reduce the rate of emergence of resistant variants. However, there are two potential gaps in the arsenal of FDA approved drugs currently used for HIV-1 treatment as follows.

First, there are no FDA approved drugs that target late events of HIV-1 replication including transcription, post-transcriptional events, assembly, particle production and virion morphogenesis, which are important stages of HIV-1 life cycle undertaken by a reactivated virus from latently infected cell. Such reactivated virus may escape the drug treatment as none of the drugs target production of the virus and these viruses may start new spreading infections.

Second, there is only one FDA approved drug (Maraviroc) (Pau and George (2014) Infect Dis Clin North Am 28:371-402) that target host-virus interactions: HIV-1 hijacks several host proteins during its replication. Many of these host factors that interact with viral proteins are essential factors for viral replication and are called “dependency factors”. Virus is defective in the absence of these host proteins. The host-virus interactions are many and distinct from each other. The host-virus interactions are specifically useful drug targets, since host does not mutate and since the interaction with host factor is essential for viral replication, if the virus develops escape mutations that renders it defective for interaction with host factor and drugs, this virus will be defective for replication. Therefore, targeting specific host-virus interactions constitutes a novel territory for development of anti-HIV drugs.

Third, there are no FDA approved drugs that target viral protein-RNA interactions. HIV-1 protein integrase interacts with HIV-1 genomic RNA and this binding is essential for its function (Kessl et al. (2012) J Biol Chem 287:16801-16811; Dixit et al. (2021) Nat Commun 12:2743). However, currently, there are no FDA approved drugs that target viral protein-nucleic acid interactions. Thus, development of a new treatment regime that would target the late events of HIV-1 replication on a patient is much needed. Furthermore, if this drug targets both host-virus and viral protein-RNA interactions, it is additionally beneficial.

### SUMMARY OF THE INVENTION

The present invention is based, at least in part, on addressing the two gaps in the HIV-1 treatment strategies and developing one or more “first-in-class inhibitors” based on the host-virus protein-protein interactions between HIV-1 integrase (IN) and host factor INI1/SMARCB1 that target the late events of HIV-1 replication. As disclosed herein, the present invention relates to the development of a new drug target in the late events of HIV-1 replication and a new treatment regime that targets host-virus interactions.

These three novel ways of targeting HIV-1, namely (1) targeting late events of HIV-1 replication, (2) targeting host-virus interactions, and (3) targeting viral RNA-protein interactions, provide mechanisms of targeting HIV-1 that are distinct from those known and practiced in the art. The distinct mechanisms of targeting HIV-1 by the inhibitors of the present disclosure have unique advantages. For example, the inhibitors described herein (1) can be used in a combination therapy with drugs that are currently FDA-approved (e.g., those targeting entry, reverse transcription, integration, or proteolytic processing), and (2) have the ability to effectively target HIV-1 that is resistant to the currently FDA-approved drugs.

Certain aspects of the invention provide compositions comprising an agent that disrupts an intracellular protein-protein interaction between IN and host factor INI1/SMARCB1 or interaction between IN and TAR RNA. In some embodiments, the agent includes, but not limited to, peptides, antibodies, small molecules, RNAs, and other modulators disclosed herein. As described herein, numerous embodiments are further provided that can be applied to any aspect of the present invention and/or combined with any other embodiment described herein. In some embodiments, the agent is a small molecule, CRISPR single-guide RNA (sgRNA), RNA interfering agent, antisense oligonucleotide, peptide or peptidomimetic inhibitor, aptamer, antibody, or intrabody. In other embodiments, the RNA interfering agent is a small interfering RNA (siRNA), CRISPR RNA (crRNA), a small hairpin RNA (shRNA), a microRNA (miRNA), or a piwi-interacting RNA (piRNA).

Other aspects of the invention provide a method of treating a subject afflicted with an HIV infection by administering to a subject a therapeutically effective dose of an agent that disrupts an intracellular protein-protein interaction between HIV-1 IN and host factor INI1/SMARCB1. The disruption of IN-INI1/SMARCB1 interaction targets late events of HIV-1 replication including assembly, particle production and particle morphogenesis.

Yet other aspects of the invention provide a method of reducing a side effect of a therapeutic regime by administering to a subject a therapeutically effective dose of an agent that disrupts an intracellular protein-protein interaction between HIV-1 IN and host factor INI1/SMARCB1, wherein the subject has received at least one therapeutic regime selected from surgery, antiretroviral therapy (ART), highly active antiretroviral therapy (HAART) or a combination thereof, and the subject is experiencing at least one side effect as a consequence of the therapeutic regime.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1A-FIG. 1E show NMR structure of Rpt1+linker domains of INI1 and molecular modeling of IN binding Rpt1+linker+Rpt2 fragment. FIG. 1A shows a schematic representation of various domains of INI1 and the inhibitory fragment S6 (WHD (in yellow)=Winged Helix DNA binding domain, DBD (in cream)=DNA binding domain; RPT (in red)=Repeat; NES (in turquoise)=Nuclear export signal; HR3 (in blue)=homology region 3; arrows represent repeats). FIG. 1B shows superposition of the residues 183-265 of the 20 lowest-energy structures of the INI1 Rpt1+linker fragment. Note the disordered nature of the linker region (aa 250-265, shown in pale pink). The helices are in green and beta sheet in blue. FIG. 1C shows ribbon diagram (in rainbow colors) of a lowest energy representative structure of Rpt1 (aa183-245). FIG. 1D shows superimposition of C-alpha atoms of five different structures (6AX5 in pink, 57LA in turquoise, 5L7B in yellow, 5GJK in magenta, 6LTJ in black) showing alignment of the Rpt1 region (aa 183-248). FIG. 1E shows ribbon diagram (in rainbow color) of a representative structure of INI1183-304 modeled using Robetta based on the NMR structure 6AX5.

FIG. 2A-FIG. 2F show molecular docking of IN-CTD with INI1₁₈₃₋₃₀₄. FIG. 2A shows ribbon diagram of bound complex of INI1₁₈₃₋₃₀₄/CTD model obtained from HADDOCK. FIG. 2B shows surface structure of INI1183-304/CTD modeled complex. FIG. 2C shows electrostatic surface; negatively charged (red), positively charged (blue) and hydrophobic (white). FIG. 2D-FIG. 2E show exploded views of the interface residues, displaying the ionic interactions (FIG. 2D), and hydrophobic non polar interactions (FIG. 2E). FIG. 2F shows representation of the regions between INI1183-304/CTD showing a hydrophobic tunnel enclosed by ionic bonds at the two ends. In all panels, INI1183-304 and its residues are shown in pink, and IN-CTD and its residues are shown in blue. Orange dotted line with arrow represents the hydrophobic tunnel.

FIG. 3A-FIG. 3C show molecular docking studies of Rpt1 INI1₁₈₃₋₃₀₄ binding to IN CTD using MDockPP. FIG. 3A shows surface representation of bound modeled complexes of CTD/Rpt1. FIG. 3B shows an interface of CTD/INI1-Rpt1 complexes. INI1-Rpt1 cartoon and residue labels are shown in pink, CTD cartoon and residues are shown in blue. FIG. 3C shows exploded view to show the presence of hydrophobic patch in the interface of IN-CTD/INI1183-304 complex structure generated by MDockPP.

FIG. 4A-FIG. 4F show particle morphology and replication of INI1-interaction defective IN mutants. FIG. 4A shows TEM analysis of wild type (WT) and R228A mutant HIV-1NL4-3 particles. Note the empty capsids with unpackaged RNP in R228A mutant. FIG. 4B shows Cryo electron tomography (CryoET) studies to demonstrate the defect in particle morphology of the virions harboring W235E mutations. Leftmost panel indicates CryoET structure of wild type (WT) particle. Therest of the panels indicate various particle morphologies observed in mutant virions. FIG. 4C shows IN binding is necessary for INI1 incorporation into HIV-1 virions. The gel images are from one out of three representative experiments. Immunoblot analysis of concentrated WT, W235E, or R228A HIV-1NL4-3 virions produced in 293 T cells. Top three panels represent immunoblot analysis of concentrated virions and the bottom four panels correspond to producer cell lysates. Western analysis was carried out using α-IN (to detect IN), α-p24 [to detect Capsid (CA, p24) and Gag (pr55)], α-BAF47 (to detect INI1) or α-GAPDH (as loading control) antibodies. FIG. 4D-4J shows analysis of replication of W235E mutant. FIG. 4D shows fluorescence microscopy images of CEM-GFP cells infected with HIV-1NL4-3 (25 ng p24 each) of WT or W235E mutant in a multiday infection. FIG. 4E shows Graphic illustration of virus particle release in the culture supernatant of the experiment in d, measured by p24 ELISA (Representative of two independent experiments). FIG. 4F shows infectivity of HIV-1-Luc reporter virus harboring either a wild type (WT) or a W235E mutant integrase. The graph represents luciferase activity of infected cells, 24 hours post-infection (n=3 independent experiments, Mean±SEM). FIG. 4G-FIG. 4I show graphic representation of effect of W235E IN mutations on earlyRT products (FIG. 4G), late RT products (FIG. 4H), and two LTR circles (FIG. 4I), as measured by qRT-PCR, at indicated times, post-infection. The data represent average of three independent experiments (n=3 independent experiments, Mean±SEM). FIG. 4J shows graphic representation of effect of W235E mutant on integration as measured by Alu-Gag PCR at 24 h post-infection. Data are compared to WT and represents average of three independent experiments (n=3 independent experiments, Mean±SEM).

FIG. 5A-FIG. 5D show molecular docking studies to compare Rpt1 INI1183-304 and TAR (trans-activation response element) binding to IN CTD. FIG. 5A shows surface representation of bound modeled complexes of CTD/TAR. FIG. 5B shows interface residues of CTD/TAR complexes. TAR cartoon and nucleotide labels are shown in reddish-orange. Note that interacting phosphate groups, bases, and sugars are shown. CTD and the interacting residues are shown in blue. FIG. 5C shows superimposition of docking complex models of IN-CTD/INI1₁₈₃₋₃₀₄ and IN-CTD/TAR generated by MDockPP. INI1 portion is indicated in pink, RNA in Orange and three-dimensional surface of CTD portion in light blue. Please note the close proximity of phosphate groups of TAR (in Dark Blue) nucleotides with negatively charged residues of INI1₁₈₃₋₃₀₄ (in Magenta), both of which are involved in interacting with CTD. FIG. 5D shows ribbon diagram showing the superimposition of Rpt1/CTD and TAR/CTD complexes.

FIG. 6A-FIG. 6E show TAR RNA mimicry of Rpt1 domain and model for role of INI1 during particle production. FIG. 6A shows Surface electrostatic computation of INI1₁₈₃₋₂₄₅ NMR structure indicating negatively (red) and positively (blue) charged and hydrophobic (white) residues. FIG. 6B and FIG. 6C show a cartoon illustrating the similarity of INI1-Rpt1 to TAR RNA. Ribbon diagram of NMR structure of INI1 Rpt1 (left) where the side chains of all 11 negatively charged residues are depicted as red spheres, and that of TAR RNA where phosphate groups of the interacting nucleotides are depicted as red spheres. FIG. 6D shows ribbon diagram of NMR structure of TAR RNA (PDB ID: 1ANR). FIG. 6E shows a model to understand the role of RNA mimicry of INI1 Rpt1 domain during HIV-1 assembly. Panel 1: In a producer cell where both INI1 and genomic RNA are present, INI1 acts as a place-holder and binds to IN portion of GagPol to prevent RNA binding to it, which otherwise may cause steric hindrance. Both RNA and INI1 are incorporated into the virions resulting in correct particle morphogenesis. Panel 2: RNA-interaction-defective and INI1-interaction-defective mutants of IN are impaired for binding to both RNA and INI1 and hence there is no steric hindrance for assembling GagPol. However, during particle maturation, lack of binding to RNA and/or INI1 could lead to morphologically defective particles, as shown in the empty conical capsid and unpackaged materials on the side of the capsid in the virion. Panel 3: Lack of INI1 leads to binding of RNA to IN portion of GagPol, which results in defective assembly and particle production. Gag (bright blue), RT (Reverse transcriptase; in blue), IN (wheat color), IN mutant (purple), INI1 (green), and HIV-1 RNA with TAR (red) are represented with the same colors both in the cells and in the virion particles, as indicated. Yellow, light green, and gray ovals represent possible INI1-binding proteins.

FIG. 7A-7C show designing of stapled peptides. FIG. 7A shows ribbon diagram showing INI1₁₈₃₋₃₀₄/IN-CTD complex. INI1183-304 is shown in pink with interface alpha helix colored in Magenta and CTD is shown in blue. FIG. 7B shows helix 1 bound to IN-CTD. FIG. 7C shows Schematic representation of stapled peptide sequences and position of stapling in peptides. SP80 is the linear peptide derived from Alpha helix 1 of INI1 Rpt-1 domain. The peptide synthesis, stapling and preparation was done by CPC Scientific San Jose, CA.

FIG. 8A-FIG. 8D show quantitative alpha interaction assay to determine the effect of stapled peptides on the interaction of IN and IN-CTD with INI1 and INI1₁₈₃₋₃₀₄ or TAR RNA. FIG. 8A shows an effect of stapled peptides on interaction of GST-CTD with 6His-SUMO-INI1₁₈₃₋₃₀₄. FIG. 8B shows an effect of stapled peptides on Interaction of GST-CTD with TAR-RNA. FIG. 8C shows Effect of stapled peptides on Interaction of GST-IN (full length) with 6His-INI1 (full length). FIG. 8D shows an effect of stapled peptides on Interaction of GST-IN (full length) with TAR-RNA. Fixed concentrations of GST-IN, 6His-INI1, and TAR RNA were incubated with increasing concentration of stapled peptides and the interaction was detected as Alpha Score. The IC50 values were determined by fitting the data to a four-parameter dose-response curve using Graph Pad prism. All the graphs represent the average of three independent experiments (Mean+/−SEM). DMSO control (no peptide), SP38, and SP39 are two different stapled peptide; SP80 linear peptide (without stapling) and SP83 stapled peptide SP38 with D225G mutation.

FIG. 8e shows inhibition of IN/TAR RNA and IN/INI1 complex formation by Stapled peptide, SP-38, in 293T cells: Panels a-c show the result of RNA-co-IP: Panel a shows the RT-PCR analysis of the bound RNA; Panel b shows the immunoblot analysis of bound HA-INI1; Panel c shows the immunoblot analysis of bound YFP-IN. Panels d-g show the input RNA and protein levels within transfected cells: the RT-PCR analysis of input TAR RNA levels (panel d); Immunoblot analysis of input HA-INI1 (panel e); input YFP-IN (panel f); and GAPDH level for loading control (panel g). Lane 1 is RNA-co-IP using isotype IgG control antibodies and lanes 2-7 are RNA-co-IP using anti-GFP antibodies to pull down IN. There is ˜1000 fold inhibition of RNA and INI1 binding to IN in the presence of SP-38 peptide (compare lane 6 to lane 5). However, the control mutant peptide, SP83 (D225G) does not inhibit RNA and INI1 binding to IN (Compare lanes 5, 6 and 7).

FIG. 9A-FIG. 9C show an inhibition of HIV-1 replication by Stapled peptides in a multiday replication assay in CEM-GFP cells. FIG. 9A shows a schematic representation of experimental plan. FIG. 9B and FIG. 9C show images representing the expression of GFP in CEM GFP cells in presence or absence of 1 and 5 micromolar Stapled peptides or DMSO control. FIG. 9D and FIG. 9E show p24 ELISA analysis of culture supernatants from CEM-GFP cells from panels FIG. 9B and FIG. 9C samples. The CEM-GFP cells were infected with HIV-1NL4-3 and treated with stapled peptides. Culture supernatants were collected on multiple days as indicated and p24 analysis was carried out.

FIG. 10A-FIG. 10B show the mechanism of HIV-1 inhibition by Stapled peptides. FIG. 10A shows schematic representation of experimental design to determine the mechanism of inhibition by stapled peptides. (1) Transfection of pNL4-3 plasmid in to 293T cells in the presence of stapled peptides and determine the particle production by analyzing p24 in the supernatant and cell lysates. (2) Virions produced are analyzed for their infectivity in the absence of stapled peptides. (3) Virions are subjected to Transmission Electron Microscopy (TEM) to determine the particle morphogenesis. (4) Purified and concentrated virions are subjected to Western blot analysis for the presence of INI1. FIG. 10B shows p24 ELISA of virions produced in the presence of stapled peptides as indicated in the panel A (1). Top panel represents p24 in virions and bottom panel shows intracellular p24 level after transfection.

FIG. 11A-FIG. 11D show virions produced in the presence of Stapled peptides are defective for infection. FIG. 11A and FIG. 11B show infectivity of virions in CEM GFP cells produced in 293T cells that were treated with various stapled peptide (at 5 μM concentration) or DMSO. Equal amounts of virions treated with stapled peptides were used to infect CEM-GFP cells and the infected cells were imaged on days 1, 3, 6, 9 and 12 (FIG. 11A) and subjected to p24 ALphaLisa (FIG. 11B). FIG. 11C and FIG. 11D show Infectivity of the virions produced in the presence of increasing concentrations of stapled peptide during production in 293T cells. The 293T cells transfected with pNL4-3 were treated with increasing concentrations of stapled peptides and equal amount of virions from these cells were used to infect CEM-GFP cells. The infected cells were imaged on day 6 (FIG. 11C) and subjected to p24 ALphaLisa (FIG. 11D).

FIG. 12A-FIG. 12C show effects of stapled peptides on virion morphogenesis and incorporation of INI1. FIG. 12A shows electron microscopy images of HIV-1 virion treated with SP38 Staple peptide. The middle panel is the expanded version and the lower panel represents the carton of the two viral particles from the middle panel. Note the empty capsid shell and eccentric electron dense material, typical of morphologically defective virions. FIG. 12B shows Immunoblot analysis of the virions produced in the presence of stapled peptides SP38 and SP83. Lane 1, Marker; Lane 2, DMSO; Lane 3, Virions produced in presence of SP83 (D225E mutant peptide); and Lane 4, Virions produced in the presence of SP38 stapled peptide. Note the lack of INI1 incorporation into virions when produced in the presence of SP38. FIG. 12C shows the quantitation of different morphological forms of virions in the presence and absence of the stapled peptides. About 200 virions each were scored to quantify four different types of morphological forms—Conical (Co), Eccentric (Ec), Immature (Im) and Unclear (Uc). Note the virions produced in the presence of SP-38 (SP38-V) showed increased proportion of eccentric virions, demonstrating that SP-38 treatment produced non-infection virions.

FIG. 13A-FIG. 13C show staple peptides inhibit infection of reactivated HIV-1 from latent cells. FIG. 13A shows schematic representation of co-culture experiment where stapled peptides are added to inhibit infection of reactivated HIV-1 from latent cells. Cells harboring latent virus (J1.1) were co-cultured with CEM-GFP cells in the presence and absence of stapled peptides. Addition of PMA reactivates HIV-1 from latent cells which infect CEM cells to induce expression of GFP. FIG. 13B shows image of J1.1+CEM GFP cells with or without PMA and Stapled peptide (5 M). Images were taken on day 2 postinduction. FIG. 13C shows graphic representation of p24 levels in co cultured cells from FIG. 13B on day 5. Experiments was done in triplicates and the graph represents mean+/−SEM. Note that SP38 inhibits p24 levels in induced and uninduced conditions.

FIG. 14A-FIG. 14F show molecular docking of IN-CTD with INI1183-304. FIG. 14A shows ribbon diagram of bound complex of INI1₁₈₃₋₃₀₄/CTD model obtained from HADDOCK. FIG. 14B shows surface structure of INI1183-304/CTD modeled complex. FIG. 14C shows electrostatic surface; negatively charged (red), positively charged (blue) and hydrophobic (white). FIG. 14D and FIG. 14E show exploded views of the interface residues, displaying the ionic interactions (FIG. 14D), and hydrophobic non polar interactions (FIG. 14E). FIG. 14F shows Representation of the regions between INI1183-304/CTD showing a hydrophobic tunnel enclosed by ionic bonds at the two ends. In all panels, INI1183-304 and its residues are shown in pink, and IN-CTD and its residues are shown in blue. Orange dotted line with arrow represents the hydrophobic tunnel.

FIG. 15A-FIG. 15C show in vitro binding studies to validate the interacting interface residues predicted in the CTD/INI1₁₈₃₋₃₀₄ complex. FIG. 15A shows of a portion of S6/Rpt1 fragment of INI1 and the IN-interaction-defective substitution mutations (E3, E4, and E10) identified in a random genetic, reverse yeast two-hybrid screen and their effect on S6-mediated inhibition of HIV-1 particle production. FIG. 15B shows GST-pull down assay to demonstrate the binding of INI1183-304 and its mutants with IN, CCD and CTD. Representative images from one out of three experiment is shown. Top panel represents bound proteins and the bottom two panels represent the loading control. Top two panels represent the Western blot using α-BAF47 antibodies to detect 6His-SUMO-INI1₁₈₃₋₃₀₄. Bottom panels represent the Coomassie-stained gel of GST-fusion proteins. FIG. 15C shows GST-pull down assay to determine the interaction of His6-IN(WT), His6-IN(W235E) mutant with GST-INI1, GST-SAP18, GST-LEDGF, and GST-Gemin2. Representative images from one out of three experiment is shown. Top panel represents the bound proteins and the two panels below the top represent the loading controls. Non-adjacent lanes from the same gel are spliced together for the figure and uncropped gels are provided in the source data. Graphs at the bottom represent quantitation of the bound proteins expressed as fraction bound after normalizing to the loading control. The graphs represent the mean of two independent experiments, WT is Wild type IN (shown in blue) and W235E shown in red.

FIG. 16A-FIG. 16H show quantitative Alpha protein-protein interaction assay to determine the interaction of IN, CTD, and the mutants with INI1 and INI1₁₈₃₋₃₀₄. FIG. 16A shows Interaction of GST-CTD with His6-SUMO-INI1183-304. A titration curve was generated with increasing concentrations of His6-SUMO-INI1₁₈₃₋₃₀₄ with two different fixed concentration of GST-CTD and the interactions were detected as Alpha Score. The KD values were determined by nonlinear regression analysis using specific binding with Hill slope analysis in GraphPad Prism. Data from one representative experiment is depicted. FIG. 16B and FIG. 16C show effect of salt on the interactions of GST-CTD either with His6-SUMO-INI1183-304 or biotinylated(Bio)-TAR RNA (n=3 independent experiments). The interaction was tested using fixed concentrations of GST-CTD (0.75 μM) and increasing concentrations of His6-SUMO-INI1₁₈₃₋₃₀₄ (or Bio-TAR RNA) in two different NaCl conditions (100 and 500 mM). FIG. 16D and FIG. 16E show inhibition of GST-CTD interaction with His6-SUMO-INI1183-304 or Bio-TAR RNA (n=6 independent experiments). Interactions were set up between GST-CTD (0.186 μM) with His6-SUMO-INI1183-304 (0.094 μM), or GST-CTD (0.03 μM) with Bio-TAR RNA (0.1 μM) and increasing concentration of the third component (indicated in the X-axis) was added to the reaction. The IC₅₀ values were determined by fitting the data to a four-parameter dose-response curve using GraphPad prism. FIG. 16F and FIG. 16H show Interaction of GST-CTD and its substitution mutants with INI1₁₈₃₋₃₀₄ and TAR RNA (n=3 independent experiments). Representative Coomassie gel (from one out of three independent experiments) showing equal loading of the wild type and mutant proteins for the binding assays (FIG. 16F) and uncropped gels are provided in the source data, Interaction of GST-CTD and mutants with INI1183-304 (FIG. 16G), and with biotinylated-TAR RNA (FIG. 16H). The graphs represent the % of the interaction of mutants as compared to that of wild type (WT) IN set at 100%. For both panels (FIG. 16G) and (FIG. 16H), WT and mutants are represented in different colors as indicated in the key provided next to the bar graphs. In all panels, except in a, graphs represent Mean±SEM. In all panels the pink cartoon Rpt1 represents INI1183-304, blue cartoon CTD, IN-CTD, and red stem-loop, TAR RNA.

FIG. 17A-FIG. 17D show INI1 competes with TAR RNA for binding to IN in vivo and facilitates particle production. a Co-immunoprecipitation of INI1 with IN and mutants in vivo. FIG. 17A shows MON cells were transfected with YFP-IN/IN mutants and HA-INI1 and then subjected to co-immunoprecipitation using α-HA antibodies. Representative images from one out of three independent experiments is shown. The top two panels illustrate results of co-immunoprecipitation using α-HA antibodies. The lower two panels represent the input control. Lanes 7-9 represent controls, use of isotype IgG antibody (lane 9) or lack of INI1 or IN (lanes 7-8). FIG. 17B shows RNA-co-IP analysis to determine the competition of INI1 and TAR RNA for binding to IN. The gel images are from one out of three representative experiments. The top three panels (i)-(iii) represent the RNA and proteins present in the immune complexes and the bottom three panels (iv)-(vi) represent proteins and RNA in the input controls. Panels (iii) and (vi), graphic representation of relative amounts of TAR RNA bound compared to control, as determined by qRT-PCR (n=3 independent experiments), normalized to control (lane 2). Immunoprecipitation was carried out using isotype IgG antibody (lane 1) or α-GFP antibodies to pull down YFP-IN (lanes 2-7). Lane 3 represents negative control without YFP-IN. Lanes #4-7 represent RNAco-IP in the presence of increasing HA-INI1. FIG. 17C shows IN-interaction-defective INI1 mutant do not compete with TAR in vivo. The gel images are from one out of three representative experiments. Panels (i)-(vi) are as in FIG. 17B. Lanes represent results of RNA-co-IP of YFP-IN with TAR RNA in the presence of WT INI1 (lane 3), INI1 (D225G) (lane 4), and INI1 (D225E) (lane 5). FIG. 17D shows INI1 binding to IN is necessary for particle production. The top panel represents virus-associated and the middle panel, the cell-associated p24, expressed as % of wild type. The bottom panel represents release efficiency as a fraction of viral and cell-associated p24 of each mutant, as compared to wild type, expressed in %. EV=Empty vector; WT=wild type. The bars represent the average of three independent experiments (Mean±SEM). The bars are color-coded as indicated in the key provided next to the graph. Uncropped gels/blots for all the images in this figure are provided in the source data.

FIG. 18A-FIG. 18J show particle morphology and replication of INI1-interaction defective IN mutants. FIG. 18A shows TEM analysis of wild type (WT) and R228A mutant HIV-1NL4-3 particles. Note the empty capsids with unpackaged RNP in R228A mutant. FIG. 18B shows cryo electron tomography (CryoET) studies to demonstrate the defect in particle morphology of the virions harboring W235E mutations. Leftmost panel indicates CryoET structure of wild type (WT) particle. The rest of the panels indicate various particle morphologies observed in mutant virions. FIG. 18C shows IN binding is necessary for INI1 incorporation into HIV-1 virions. The gel images are from one out of three representative experiments. Immunoblot analysis of concentrated WT, W235E, or R228A HIV-1NL4-3 virions produced in 293 T cells. Top three panels represent immunoblot analysis of concentrated virions and the bottom four panels correspond to producer cell lysates. Western analysis was carried out using α-IN (to detect IN), α-p24 [to detect Capsid (CA, p24) and Gag (pr55)], α-BAF47 (to detect INI1) or α-GAPDH (as loading control) antibodies. Representative of two independent experiments. Uncropped blots of this Western analysis are provided in the source data. FIG. 18D-FIG. 18J show analysis of replication of W235E mutant. FIG. 18D shows fluorescence microscopy images of CEM-GFP cells infected with HIV-1NL4-3 (25 ng p24 each) of WT or W235E mutant in a multiday infection. FIG. 18E shows graphic illustration of virus particle release in the culture supernatant of the experiment in d, measured by p24 ELISA (Representative of two independent experiments). FIG. 18F shows Infectivity of HIV-1-Luc reporter virus harboring either a wild type (WT) or a W235E mutant integrase. The graph represents luciferase activity of infected cells, 24 hours post-infection (n=3 independent experiments, Mean±SEM). FIG. 18G-FIG. 181 show graphic representation of effect of W235E IN mutations on early RT products (FIG. 18G), late RT products (FIG. 19H), and two LTR circles (FIG. 181), as measured by qRT-PCR, at indicated times, post-infection. The data represent average of three independent experiments (n=3 independent experiments, Mean±SEM). FIG. 18J shows graphic representation of effect of W235E mutant on integration as measured by Alu-Gag PCR at 24 h post-infection. Data are compared to WT and represents average of three independent experiments (n=3 independent experiments, Mean±SEM).

FIG. 19A-FIG. 19F show molecular docking studies to compare Rpt1 INI1183-304 and TAR binding to IN CTD. FIG. 19A and FIG. 19C show surface representation of bound modeled complexes of CTD/Rpt1 and CTD/TAR. FIG. 19B and FIG. 19D show interface CTD/INI1-Rpt1 and CTD/TAR complexes. INI1-Rpt1 cartoon and residue labels are shown in pink, CTD cartoon and residue labels are shown in blue and TAR cartoon and nucleotide labels are shown in reddish-orange. Note that interacting phosphate groups, bases, and sugars are shown. FIG. 19E shows orientations of the key residues on CTD after docking shown in magenta (interacting with INI1-Rpt1) or black (interacting with TAR). FIG. 19F shows Superimposition of the CTD/Rpt1 and CTD/TAR complexes shows the identical orientation of CTD and nice overlap of Rpt1 and TAR RNA regions. In all panels, IN-CTD is represented in bright blue, INI1-Rpt1 in pink, TAR RNA in orange colors respectively.

FIG. 20A-FIG. 20B show cloning, expression and purification of overlapping fragments of INI1 containing Rpt1. FIG. 19A shows cartoon representing various overlapping fragments of INI1. Numbers below the bars represent amino acid residue positions. The top bar represents full length INI1, and the bars 1-5 below the top bar represents INI1 fragments. Number in the parentheses indicate clone numbers. WHD (in yellow)=Winged Helix DNA binding domain; DBD (in cream)=DNA Binding Domain; RPT (in red)=Repeat; NES (in turquoise)=Nuclear Export Signal; HR3 (in blue)=homology region 3; and arrows represent repeats. FIG. 19B shows Coomassie stained SDS/PAGE gel indicating purified INI1 fragments from one out of three independent experiments. The fragments were cloned as His₆-SUMO-fusions and were purified in two steps using Ni-NTA column as described in the methods for purification of INI1₁₈₃₋₂₆₅. Lane numbers correspond to the fragments in FIG. 20A.

FIG. 21A-FIG. 21B show purification of INI1183-265 fragment. FIG. 21A shows schematic representation of INI1 (top bar) and INI1 fragments, the transdominant negative mutant S6(INI1₁₈₃₋₂₉₄), INI1₁₈₃₋₂₆₅ fragment, and the Sumo fusion of INI1₁₈₃₋₂₆₅ fragment. Numbers below the bar represent amino acid positions. WHD (in yellow) Winged Helix DNA binding domain; DBD (in cream)=DNA Binding Domain; RPT (in red)=Repeat; NES (in turquoise)=Nuclear Export Signal; HR3 (in blue)=homology region 3; and arrows represent repeats. FIG. 21B shows Coomassie gel indicating proteins from various stages of purification of INI1₁₈₃₋₂₆₅ as indicated in the labels for the lanes (representative gels from one out of three independent experiments is shown). The left panel indicates two-step purification of INI1₁₈₃₋₂₆₅. The right panel indicates peak fractions collected from subsequent gel filtration chromatography. The numbers above the lanes indicate fraction numbers. The graph below the right panel indicates the absorbance (at 280 and 260 nm) of the fractions collected from the gel filtration column.

FIG. 22A-FIG. 22B show ¹H¹⁵N HSQC NMR spectrum of 1 mM INI1₁₈₃₋₂₆₅ and analytical Ultra Centrifugation of INI1₁₈₃₋₂₆₅. FIG. 22A shows time-derived distribution of INI1₁₈₃₋₃₀₄ determined as described in FIG. 22B. FIG. 22B shows analytical ultracentrifugation. The crosses denotes the data points. The line denotes the best fit to the noninteracting single component model. Forty four of the 120 sedimentation scans were used to calculate the time-derivative distribution. INI1₁₈₃₋₂₆₅ sediments as a single component characterized by S_{20,w}=1.035 (1.033, 1.038) S and an apparent molecular weight (S/D)_{20,w}=9.92 (9.55, 10.09) kDa. The diffusion coefficient corresponding to the best fit molecular mass is 9.66 F. Since the molecular weight calculated from the sequence of INI1₁₈₃₋₂₆₅ is 9.566 kDa, we conclude that under the solution conditions analyzed this protein is monomeric.

FIG. 23A-FIG. 23F show in vitro binding of IN and its domains with INI1 and its fragments. FIG. 23A shows cartoon representing various INI1 fragments (1-6) as in FIG. 20a, all of which were expressed as His₆-SUMO fusions and used for in vitro binding with IN as in FIGS. 23B and 23C. FIG. 23B shows results of in vitro binding of various His₆-SUMO-INI1 fragments with GST-IN. Top panel: Western blot of lysates expressing INI1 fragments used for binding experiment; Middle panel: Loading control for GST-IN and GST proteins; Bottom panel: Bound INI1 fragments. Note the positive interaction of INI1₁₈₃₋₃₀₄ fragment in the last lane. FIG. 23C-23D show binding of purified INI1₁₈₃₋₃₀₄ and INI1₁₆₆₋₃₀₄ fragments with GST-fusion of IN and domains: GST-pull down assay was carried out as described in the methods and bound proteins were detected using α-BAF47 antibodies. FIG. 23C shows Coomassie blue stained gel of INI1₁₈₃₋₃₀₄ and INI1₁₆₆₋₃₀₄ proteins loading control. FIG. 23D shows (top panel) Coomassie blue stained gel of GST fusions of IN, central core, C-terminal and N-terminal domains (CCD, CTD and NTD) as loading control. Bottom panel, Immunoblot of the bound proteins, IN1₁₈₃₋₃₀₄ and IN1₁₆₆₋₃₀₄ fragments. FIG. 23E-23F show in vitro binding of domain of IN with full-length INI1. FIG. 23E shows cartoon representing IN and its three domains. FIG. 23F shows, top panel, Immunoblot of the bound INI1. Bottom panel, GST-fusions of IN and domains with His₆-INI1 as input control. The “*” indicates the position of full length protein in each case. In the panels FIGS. 23B-23D and 23F, representative images from one out three independent experiments are shown. “MW” is molecular weight markers.

FIG. 24A-FIG. 24D show modeling of INI1₁₈₃₋₃₁₉ fragment containing Rpt1-linker-Rpt2 regions and validation of INI1₁₈₃₋₃₀₄ model. FIG. 24A shows ribbon diagram representing the superimposition of representative INI1183-319 models from five different clusters obtained from Robetta. These five models are color coded in pink, yellow, turquoise, brown and green colors. Note that while Rpt1 region of each model is perfectly aligned, the spatial positioning of the Rpt2 in relation to Rpt1 is altered due to the differential folding of the linker region. While two Rpt2 models (brown and green) occupy space right of the Rpt1 the other three Rpt2 models (pink, yellow and turquoise), the left side of Rpt1. FIG. 24B-24D show validation of the modeling of INI1₁₈₃₋₃₀₄ structure. FIG. 24B shows the Ramachandran plot indicating the presence of 90.1% and 9.9% residues in the favored and allowed regions, and 0% residues in the outlier region, respectively. FIG. 24C shows profile 3D plot (using VERIFY3d v3.1) to determine how well the structure is folded in the 3D space. FIG. 24D shows Z score plot (obtained using ProSA 2003) to determine the energetics of the modelled protein.

FIG. 25A-FIG. 25B show an exploded view of T214 residue in the IN-CTD/INI1₁₈₃₋₃₀₄ complex structure; and In vitro binding of His₆-fusions of IN and mutants W235E, W235F and W235K with GST-INI1. FIG. 25A shows INI1-Rpt1 portion and its residues are indicated in pink and IN-CTD portion and its residues are indicated in blue. FIG. 25B shows the in vitro binding assay was carried out by using the bacterial lysates expressing His₆-TN or its mutants and GST-INI1 immobilized on G-beads. The top panel represents the Western blot of the bound proteins, middle panel represents Western blot of the input control of His₆-IN fusion proteins and the bottom panel represents the input control of the GST-INI1 fusion proteins. (Please note: Uncropped gels are provided in the Supplementary data files).

FIG. 26A-FIG. 26B show an Alpha assay to determine the interaction between full length IN/INI1 and Competition of IN-CTD and INI1₁₈₃₋₃₀₄ interaction with viral RNA nucleotides, ntd (237-279). FIG. 26A shows interaction of His₆-Sso7d-IN with GST-INI1. The graph represents one representative experiment. Increasing concentrations of GST-INI1 were incubated with fixed concentration of His₆-Sso7d-IN and the interactions were detected as Alpha Score. The K_{D} values were determined by nonlinear regression analysis using specific binding with Hill slope analysis in GraphPad Prism. FIG. 26B shows increasing concentration of HIV-1 viral RNA fragment from the region 237-279 was used to test its effect on binding of IN-CTD with INI1₁₈₃₋₃₀₄. Note that the IN-CTD/INI1₁₈₃₋₃₀₄ interactions were not significantly inhibited by HIV-1 RNA ntd (237-279). The graph is derived from plotting values from six independent experiments (n=6 independent experiments). The graph represents Mean+/−SEM.

FIG. 27A-FIG. 27B show quantitation of virus particle morphology of wild type and mutant IN virions. FIG. 27A shows graphical representation of % of normal (conical) and abnormal particles in the Transmission Electron Microscopy analysis of WT and W235E mutant. While the “conical” in the legend represents the wild type virion particles, “empty shell” represents the lack of electron dense material within the capsid core and presence of eccentric accumulation of electron dense materials. (WT, n=103 particles; and R228A n=186 particles). FIG. 27B shows a table depicting the % of various normal and defective particles identified in WT and W235E mutant using the CryoET analysis.

FIG. 28A-FIG. 28B show modeling of IN-CTD/INI1183-304 and IN-CTD/TAR RNA interactions using MDockPP. FIG. 28A shows ribbon diagram of the exploded view of IN-CTD/INI1₁₈₃₋₃₀₄ complex structure model generated by MDockPP to show the presence of hydrophobic patch in the interface residues. INI1 portion is indicated in pink and INI1 residues in green; and CTD portion is indicated in blue and CTD residues in red. FIG. 28B shows superimposition of docking complex models of IN-CTD/INI1₁₈₃₋₃₀₄ and IN-CTD/TAR generated by MDockPP. INI1 portion is indicated in pink, RNA in Orange and three-dimensional surface of CTD portion in light blue. Please note the close proximity of phosphate groups of TAR (in Dark Blue) nucleotides with negatively charged residues of INI1₁₈₃₋₃₀₄ (in Magenta), both of which are involved in interacting with CTD.

For any figure showing a bar histogram, curve, or other data associated with a legend, the bars, curve, or other data presented from left to right for each indication correspond directly and in order to the boxes from top to bottom of the legend.

### DETAILED DESCRIPTION OF THE INVENTION

The present invention is based, at least in part, on addressing the two gaps in the HIV-1 treatment strategies and developing one or more “first-in-class inhibitors” based on the host-virus protein-protein interactions between HIV-1 integrase (IN) and host factor INI1/SMARCB1 that target the late events of HIV-1 replication. As disclosed herein, the present invention relates to the development of a new drug target in the late events of HIV-1 replication and a new treatment regime that targets host-virus interactions.

Certain aspects of the invention provide a method of treating a subject afflicted with an HIV infection by administering to a subject a therapeutically effective dose of an agent that disrupts an intracellular protein-protein interaction between HIV IN and host factor INI1/SMARCB1. The disruption of IN-INI1/SMARCB1 interaction targets late events of HIV-1 replication including assembly, particle production and particle morphogenesis.

Other aspects of the invention provide a method of reducing a side effect of a therapeutic regime by administering to a subject a therapeutically effective dose of an agent that disrupts an intracellular protein-protein interaction between HIV IN and host factor INI1/SMARCB1, wherein the subject has received at least one therapeutic regime selected from surgery, antiretroviral therapy (ART), highly active antiretroviral therapy (HAART) or a combination thereof, and the subject is experiencing at least one side effect as a consequence of the therapeutic regime.

Yet other aspects of the invention provide a composition, comprising a peptide having at least about 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 1 (lys-Leu-Met-Thr-Pro-Glu-Met-Phe-Ser-Glu-Ile-Leu-Cys-Asp-Leu-Asn); and/or a peptide having at least about 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO. 1 (lys-Leu-Met-Thr-Pro-Glu-Met-Phe-Ser-Glu-Ile-Leu-Cys-Asp-Leu-Asn) and one or more unnatural amino acids, or a pharmaceutically acceptable salt, a metabolite, or a carrier thereof. In some embodiments, the peptide is stapled by forming a covalent linkage between the side-chains of the one or more unnatural amino acids and/or the one or more unnatural amino acids comprises (S)-2-(4-pentenyl) alanine and (R)-2-(7-octenyl) alanine.

Yet other embodiments, at least two unnatural amino acids are present within the peptide, and wherein the at least two unnatural amino acids are located within about 1, 2, 3, 4, 5, 6, 7, 8, 9, 10, 11, 13, 14, or 15 amino acid apart from each other. In other embodiments, the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 2 (lys-Leu-Met-Thr-Pro-Glu-Met-Phe-S5-Glu-Ile-Leu-S5-Cys-Asp-Leu-Asn), wherein S5 is (S)-2-(4-pentenyl) alanine. In other embodiments, the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 3 (lys-Leu-Met-Thr-Pro-Glu-R8-Met-Phe-Glu-Ile-Leu-S5-Cys-Asp-Leu-Asn), wherein R8 is (R)-2-(7-octenyl) alanine and S5 is (S)-2-(4-pentenyl) alanine. Yet other embodiments, the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 4 (lys-Leu-Met-Thr-Pro-Glu-Met-Phe-S5-Glu-Ile-Leu-S5-Cys-Gly-Leu-Asn), wherein S5 is (S)-2-(4-pentenyl) alanine. The composition according to any one of the preceding embodiments, wherein the peptide is in an alpha-helical conformation or in a linear conformation. In some embodiments, the peptide disrupts the interaction between human immunodeficiency virus integrase (IN) and INI1/SMARCB1 complex and/or the interaction between IN and trans-activation response element (TAR) RNA. In some embodiments, the composition according to any one of preceding embodiments further comprises at least one antiretroviral agent.

Further, the present invention provides methods of treating an HIV infection, comprising: administering to a subject a therapeutically effective dose of the composition according to any one of the preceding embodiments or aspects, or a pharmaceutically acceptable salt, a metabolite, or a carrier thereof. The subject can be a human, an animal, a cell, or a tissue. In accordance with the invention, the HIV infection comprises, but not limited to, conditions and/or diseases associated with an HIV-infection, acquired immunodeficiency syndrome (AIDS), or a combination thereof. Consistent with these embodiments, the composition is administered orally, subcutaneously, intramuscularly, or intravenously.

Yet other aspects of the invention provide methods of reducing a side effect of a therapeutic regime, comprising: administering to a subject a therapeutically effective dose of the composition according to any one of the preceding embodiments or a pharmaceutically acceptable salt, a metabolite, a carrier thereof, wherein: the subject has received at least one therapeutic regime selected from surgery, antiretroviral therapy (ART), highly active antiretroviral therapy (HAART) or a combination thereof, and the subject is experiencing at least one side effect as a consequence of the therapeutic regime. In accordance with these embodiments or aspects, the subject has previously been or can concurrently being treated with at least one antiretroviral agent including, but not limited to, zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, vivecon, and combinations thereof. In some embodiments, the side effect is selected from drug-resistance, relapse, retention of HIV-infected lymphocytes, generation of a viral reservoir, and combinations thereof. Yet in other embodiments, the subject is a human or an animal.

In further aspects of the invention include a method of treating an HIV infection, comprising: administering to a subject at least one therapeutically effective dose of an agent that inhibits, modulates, or disrupts the interaction between IN and INI1/SMARCB1 complex and/or the interaction between IN and TAR RNA, wherein the agent comprises any one of peptides, RNAs, antibodies, and/or small molecules as disclosed herein specification, figures, and/or tables. Yet in some embodiments, the composition or the agent disclosed herein, partially or completely, inhibits the generation of infectious HIV particles, lowers the infectivity of HIV, inhibits spread of HIV, and/or inhibits the infection by the reactivated virus from latent cells.

Current antiretrovirals target reverse transcription, integration, entry and maturation. But there are no known drugs that can target assembly and particle production. The present invention provides new class of agents or drugs that can target assembly, particle production and particle maturation. Also, there are no FDA approved drugs that disrupt the interaction of integrase with host factor. Agents and/or drugs as disclosed herein target the integrase/Gag-Pol and host factor interaction and there are no drugs that have dual activity to target both viral-host protein-protein and viral-viral protein-RNA interactions. These agents and/or drugs target one or both of these interactions as it is derived from the novel principle of RNA mimicry of the INI1 with viral TAR RNA. As disclosed herein, the present invention provides the potential agents or drugs to target reactivated HIV-1 latent reservoir as it inhibits late events of HIV-1 replication. These agents or drugs can be used in combination with LRA in a “shock and kill” therapy, where latent virus is activated with LRA and allowed to produce virus. Accordingly, the virus that is produced will be defective in morphology when agents, drugs, or compositions in accordance with the present disclosure and hence will inhibit the spreading infection. Further, the agents, drugs, and/or compositions as disclosed herein have the ability to overcome the problem associated with resistant mutants. As disclosed herein, the genetic studies suggest that escape mutants of IN that are defective for binding to INI1 inside the cells are able to produce particles but are blocked at another step during the replication, that is particle morphogenesis and infection. This property makes this agent/drug/composition as disclosed herein attractive as it reduces the probability of emergence of resistant mutants.

### Definitions

The articles “a” and “an” are used herein to refer to one or to more than one (i.e., to at least one) of the grammatical object of the article. By way of example, “an element” means one element or more than one element.

The term “activity” when used in connection with proteins or protein complexes means any physiological or biochemical activities displayed by or associated with a particular protein or protein complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes ability to maintain the form of protein complex), antigenicity and immunogenicity, enzymatic activities, etc. Such activities may be detected or assayed by any of a variety of suitable methods as will be apparent to skilled artisans.

The term “administering” is intended to include modes and routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the nonhuman antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

“Antiretroviral therapy agents” or “antiretroviral agents” can include, but are not limited to, zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, and vivecon, or any combination thereof.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In some embodiments, the control comprises obtaining a “control sample” from which expression product levels are detected and compared to the expression product levels from the test sample. Such a control sample may comprise any suitable sample, including but not limited to a sample from a control HIV patient (can be stored sample or previous sample measurement) with a known outcome; normal tissue or cells isolated from a subject, such as a normal patient or the HIV patient, cultured primary cells/tissues isolated from a subject such as a normal subject or the HIV patient, adjacent normal cells/tissues obtained from the same organ or body location of the HIV patient, a tissue or cell sample isolated from a normal subject, or a primary cells/tissues obtained from a depository. In preferred embodiments, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care antiretroviral therapy (ART), highly active antiretroviral therapy (HAART)). It will be understood by those of skill in the art that such control samples and reference standard expression product levels can be used in combination as controls in the methods of the present invention. In some embodiments, the control may comprise normal or infected cell/tissue sample. In preferred embodiments, the control may comprise an expression level for a set of patients, such as a set of HIV patients, or for a set of HIV patients receiving a certain treatment, or for a set of patients with one outcome versus another outcome. In the former case, the specific expression product level of each patient can be assigned to a percentile level of expression, or expressed as either higher or lower than the mean or average of the reference standard expression level.

The term “determining a suitable treatment regimen for the subject” is taken to mean the determination of a treatment regimen for a subject that is started, modified and/or ended based or essentially based or at least partially based on the results of the analysis according to the present invention. One example is starting an adjuvant therapy after surgery whose purpose is to decrease the risk of recurrence. The determination can, in addition to the results of the analysis according to the present invention, be based on personal characteristics of the subject to be treated. In most cases, the actual determination of the suitable treatment regimen for the subject will be performed by the attending physician or doctor.

A molecule is “fixed” or “affixed” to a substrate if it is covalently or non-covalently associated with the substrate such that the substrate can be rinsed with a fluid (e.g., standard saline citrate, pH 7.4) without a substantial fraction of the molecule dissociating from the substrate.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the phrase “located within about 1, 2, 3, 4, 5, 6, 7, 8, 9, 10, 11, 13, 14, or 15 amino acid apart from each other” means the amino acids are separated by the number of amino acids indicated therein. For example, if X and Y are located within 2 amino acids apart from each other, then there are two amino acids (AA) between X and Y, i.e., X-AA-AA-Y.

The term “mode of administration” includes any approach of contacting a desired target (e.g., cells, a subject) with a desired agent (e.g., a therapeutic agent). The route of administration, as used herein, is a particular form of the mode of administration, and it specifically covers the routes by which agents are administered to a subject or by which biophysical agents are contacted with a biological material.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

In yet other embodiments, the agent is a small molecule inhibitor, CRISPR single-guide RNA (sgRNA), RNA interfering agent, antisense oligonucleotide, peptide or peptidomimetic inhibitor, aptamer, antibody, or intrabody. In other embodiments, the RNA interfering agent is a small interfering RNA (siRNA), CRISPR RNA (crRNA), a small hairpin RNA (shRNA), a microRNA (miRNA), or a piwi-interacting RNA (piRNA).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In some embodiments, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. “Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In some embodiments, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA). In other embodiments, an siRNA is a small hairpin (also called stem loop) RNA (shRNA). In some embodiments, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

The term “subject” refers to any healthy animal, mammal or human, or any animal, mammal or human afflicted with an HIV. The term “subject” is interchangeable with “patient.”

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically-effective amount” and “effective amount” as used herein means that amount of a compound, material, or composition comprising a compound encompassed by the present invention which is effective for producing some desired therapeutic effect in at least a sub-population of cells in an animal at a reasonable benefit/risk ratio applicable to any medical treatment. Toxicity and therapeutic efficacy of subject compounds can be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to administration of a suitable control agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to administration of a suitable control agent.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, nonionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra). Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a cater material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate. Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits or enhances) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel. Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. U.S.A. 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In some embodiments, an agent encompassed by the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays.

“Treat,” “treating” or “treatment” of any disease refers to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treat,” “treating” or “treatment” also refers to inhibiting the disease, either physically, (e.g., stabilization of a discernible symptom), physiologically, (e.g., stabilization of a physical parameter), or both, and to inhibiting at least one physical parameter that can or cannot be discernible to the subject. In certain embodiments, “treat,” “treating” or “treatment” refers to delaying the onset of the disease or at least one or more symptoms thereof in a subject which can be exposed to or predisposed to a disease even though that subject does not yet experience or display symptoms of the disease.

TABLE 1
Statistics for the 20 Lowest Energy NMR Structures of INI1₁₈₃₋₂₆₅.

| | |
|---|---|
| <SA>^{a} | SA_{lowest} |

Experimental restraints^{b}

| | | |
|---|---|---|
| All, Å (1754) | 0.122 ± 0.01 | 0.137 |
| Intraresidue (515) | 0.006 ± 0.006 | 0.075 |
| Sequential (428) | 0.197 ± 0.025 | 0.23 |
| Short (339) | 0.035 ± 0.007 | 0.028 |
| Long (382) | 0.136 ± 0.007 | 0.139 |
| Ambiguous (90) | 0.003 ± 0.007 | 0.001 |
| Hydrogen-bond restraints, Å (58) | 0.013 ± 0.003 | 0.126 |
| Dihedral-angle restraints, ° (116) | 0.63 ± 0.39 | 0.52 |
| NOE violations >0.4 Å | 10.7 ± 2.8 | 11 |
| Angle violations >3° | 1.6 ± 1.1 | 2 |

Deviations from idealized covalent geometry^{c}

| | | |
|---|---|---|
| Bonds, Å (1348) | 0.007 ± 0.001 | 0.007 |
| Angles, ° (2457) | 0.7 ± 0.01 | 0.68 |
| Improper dihedrals, ° (666) | 0.97 ± 0.19 | 0.89 |

Structural statistics for the ensemble^{d}

| | | |
|---|---|---|
| PROCHECK parameters | | |
| Most-favored region, % | 87.9 ± 2.5 | 89.3 |
| Additionally allowed, % | 9.7 ± 2.2 | 9.3 |
| Generously allowed, % | 0.9 ± 1.4 | 0.0 |
| Disallowed, % | 1.5 ± 1.2 | 1.3 |
| Number of bad contacts | 0.2 ± 0.4 | 0 |

RMSD from the average structure^{E}

| | | |
|---|---|---|
| Backbone (N, Ca, C), Å | 0.49 ± 0.11 | 0.41 |
| Heavy atoms, Å | 0.93 ± 0.11 | 0.87 |

^{a}<SA> represents the set of 20 selected conformers obtained by restrained dynamical simulated annealing refined in a box of water in CNS/Xplor-NIH. SA_{lowest} refers to the lowest energy structure of the set.
^{b}Sum averaging of NOE distance restraints was used for groups with degenerate proton chemical shifts. The interproton unambiguous distance restraint list comprised 515 intraresidue, 428 sequential (|i − j| = 1|), 339 short range (1 < |i − j| < 5), 382 long-range (|i − j| > 5) and 90 ambiguous. Hydrogen-bonds restraints were applied as pairs of distance restraints: HNÂ•Â•Â•O, 1.2-2.2 Å; NÂ•Â•Â•O, 1.2-3.2 Å. The final values for the respective force constants were: NOE, 30 kcal mol − 1 Å − 2; H-bonds, 50 kcal mol − 1 Å − 2; dihedral angles, 200 kcal mol − 1 rad − 2.
^{c}The final values for the respective force constants were: bond lengths, 500 kcal mol − 1 Å − 2; angles and improper torsions, 500 kcal mol − 1 rad − 2; the improper torsion angle restraints serve to maintain planarity and chirality.
^{d}The program PROCHECK (Laskowski et al., 1996) was used to assess the stereochemical parameters of the family of conformers for Ini1. The figures indicate the percentage of residues with backbone φ and ψ angles in separate regions of the Ramachandran plot, defined in the program. The number of bad contacts per 100 residues is expected to be in the range 0-30 for protein crystal structures of >3.0-Å resolution.
^{e}The precision of the atomic coordinates is defined as the average pair-wise rmsd between each of the 20 conformers and a mean coordinate structure SA generated by iterative best-fit of the backbone atoms (N, Ca, and C) over residues 183-248 of Ini1 (omitting the flexible C termini residues), followed by coordinate averaging.

TABLE 2
RMSDs of other NMR and crystal structures
as compared to 6AX5 (residues 188-246)

| | Cα RMSD | Backbone RMSD | Heavyatom RMSD | |
|---|---|---|---|---|
| PDB | (Å) | (Å) | (Å) | Method |
| 5L7B | 1.20 | 1.25 | 1.98 | NMR |
| 5L7A | 0.99 | 1.04 | 1.78 | crystal |
| 5GJK | 1.24 | 1.29 | 1.83 | crystal |
| 6LTJ | 1.12 | 1.15 | 1.77 | CryoEM |

TABLE 3
RMSDs between the Rpt1 structure generated by Robetta and
other experimentally resolved structures (residues 188-246)

| | Cα | Backbone | Heavyatom | |
|---|---|---|---|---|
| | RMSD | RMSD | RMSD | |
| PDB | (Å) | (Å) | (Å) | Method |
| 6AX5 | 1.11 | 1.16 | 1.93 | NMR |
| 5L7B | 0.76 | 0.79 | 1.70 | NMR |
| 5L7A | 0.80 | 0.85 | 1.51 | crystal |
| 5GJK | 0.76 | 0.77 | 1.34 | crystal |
| 6LTJ | 0.80 | 0.84 | 1.49 | CryoEM |

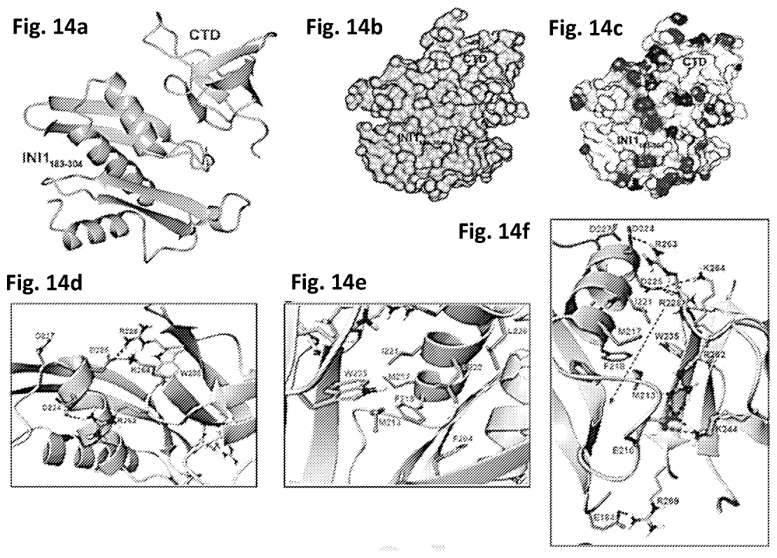

TABLE 4
Comparison of the MDockPP result and the Haddock result
(FIG. 14) of docking INI1₁₈₃₋₃₀₄ with IN CTD.

| | MDockPP | Haddock |
|---|---|---|
| ITScorePP score | −359.2 | −207.3 |
| Total buried surface area (Å²) | 865.0 | 642.2 |
| Steric clashes | 0 | 0 |
| Electrostatics | good match | good match |

TABLE 5
MDockPP results of docking TAR RNA with IN CTD.

| | MDockPP |
|---|---|
| ITScorePP score | −380.5 |
| Total buried surface area (Å²) | 826.0 |
| Steric clashes | 0 |
| Electrostatics | |

TABLE 6
A list of primers and Oligonucleotide
Table 6a:
Primers used for cloning INI1₁₈₃₋₂₆₅, INI1₁₈₃₋₃₀₄ and IN fragments

| | |
|---|---|
| Primers | VFOR-5′-TCCGAATTCGAGCTCCGTCGACAAGC-3′ |
| for | VREV-5′-TCCACCAATCTGTTCTCTGTGAGCCTC-3′ |
| amplifying | |
| the Vector | |
| Primers | S6(Rpt1)-For 5′-GGCTCACAGAGAACAGATTGGTGGATCC ccc gag gtg |
| for | ctg gtc ccc atc cgg ctg-3′ |
| amplifying | INII(aa 265)-Rev 5′-GCTTGTCGACGGAGCTCGAATTCGGATTA cta ctt |
| INI1₁₈₃₋₂₆₅ | gat gat gac gcg ctg gtc tga--3′ |
| and | INII(aa 304)-Rev 5′-GCTTGTCGACGGAGCTCGAATTCGGATTA gcc |
| INI1₁₈₃₋₃₀₄ | caa ccc cag ctc cga gca cag ctt cag-3′ |
| fragments | |

Table 6b:
Primers used for mutagenesis of INI1₁₈₃₋₃₀₄ and pCGN-INI1

| Clone | Primer sequence |
|---|---|
| E3(D225G) | Forward: 5′GCGGGTTCAAATCCAGACCGTCACAGAGGATTTCT-3′ |
| | Reverse: 5′-AGAAATCCTCTGTGACGGTCTGGATTTGAACCCGC-3′ |
| E4(T214A) | Forward-CATGAATGAGAAGTTGATGGCGCCTGAGATGTTTTCAGA-3′ |
| | Reverse 5-TCTGAAAACATCTCAGGAGCCATCAACTTCTCATTCATG-3′ |
| E10(D227G) | Forward-5′-CGTCAGCGGGTTCAAACCCAGATCGTCACAGAG-3′ |
| | Reverse-5′CTCTGTGACGATCTGGGTTTGAACCCGCTGACG-3′ |
| D225E | Forward 5′-GAAATCCTCTGTGACGAGCTGGATTTGAACCCGCT-3′ |
| | Reverse 5′-AGCGGGTTCAAATCCAGCTCGTCACAGAGGATTTC-3′ |

Table 6c:
Primers used for mutagenesis of IN-CTD

| Clone | Primer sequence |
|---|---|
| IN(W235F | Forward: 5′- |
| | TTTGCTGGTCCTTTGAAAAGTGGATTTCTGCTGTCCCTGTA-3′; |
| | Reverse: 5′ |
| | TACAGGGACAGCAGAAATCCACTTTTCAAAGGACCAGCAAA-3′ |
| IN-CTD | Forward: 5′- |
| (W235E) | GACCTTTGCTGGTCCTTTCTCAAGTGGATTTCTGCTGCTC-3′; |
| | Reverse: |
| | 5′GGACAGCAGAAATCCACTTGAGAAAGGACCAGCAAAGCTC-3′ |
| IN-CTD | Forward: 5′- |
| (W235A) | GAGCTTTGCTGGTCCTTTCGCAAGTGGATTTCTGCTGCTC-3′; |
| | Reverse: 5′- |
| | GGACAGCAGAAATCCACTTGCGAAAGGACCAGCAAAGCTC-3′ |
| IN-CTD | Forward: 5′- |
| (W235K) | GGACAGCAGAAATCCACTTAAGAAAGGACCAGCAAAGCTC-3′; |
| | Reverse: 5′- |
| | GAGCTTTGCTGGTCCTTTCTTAAGTGGATTTCTGCTGCTC-3′ |
| IN-CTD | |
| (W235F) | |
| IN-CTD | Forward-5′ |
| (K264A/ | CCATCTGTTTTCCATAATCCCTAATGATCGCTGCTGCTCTTCTTGGC |
| K266A) | ACTACTTTTATGTCACTAT-3′; |
| | Reverse-5′ATAGTGACATAAAAGTAGTGCCAAGAAGAGCAGCAGCGATCATT |
| | AGGGATTATGGAAAACAGATGG-3′ |
| IN-CTD | Forward-5′- |
| (K269A/ | CACCTGCCATCTGTGCTCCATAATCCGCAATGATCTTTGCTTTTCTT |
| K273A) | CTTGGCAC-3′ |
| | Reverse-5′- |
| | GTGCCAAGAAGAAAAGCAAAGATCATTGCGGATTATGGAGCACAG |
| | ATGGCAGGTG-3′ |
| IN-CTD | Forward:-5′ CAA AGT GGA TTT CTG CTG TCC GCG TAA TAA ACC |
| (R228A) | CGA AAA TTT TGA ATT TTT G-3′ |
| | Reverse:-CAA AAA TTC AAA ATT TTC GGG TTT ATT ACG CGG ACA |
| | GCA GAA ATC CAC TTT G-3′ |
| IN-CTD | Forward:-CTA CTG CCC CTT CAC CTG CCC AGA GGA GCT TTG CTG |
| (K244A) | Reverse:-CAG CAA AGC TCC TCT GGG CAG GTG AAG GGG CAG |
| | TAG |

Table 6d:
Sequence of the TAR RNA oligonucleotide

| RNAS | Sequence |
|---|---|
| Biotin | 5′-biotin dT/GGUCUCUCUGGU |
| labelled | UAGACCAGAUCUGAGCCUGGGAGCUCUCUGGCUA |
| TAR | ACUAGGGAACC/3′-biotin dT |
| (HIV-1vRNA | |
| (1-57)- | |
| BIO-TAR) | |
| Unlabeled | 5′ GGUCUCUCUGGUUAGACCAGA |
| TAR | UCUGAGCCUGGGAGCUCUCUGGCUAACUAGGG AACC 3′ |
| (HIV-1VRNA | |
| (1-57)- | |
| TAR): | |
| Unlabeled | 5′ GCAGGACUCGGCUUGCUGAA |
| Control | GCGCGCACGGCAAGAGGCGAGGG 3′ |
| RNA | |
| (HIV-1VRNA | |
| (237-279): | |

Table 6e:
primers and probes used to detect various stages of HIV-1 replication

| | |
|---|---|
| Early | ert2f 5′-GTGCCCGTCTGTTGTGTGAC-3′, |
| RT | ert2r 5′-GGCGCCACTGCTAGAGATTT-3′, and |
| primers | ert2 probe 5′-CTAGAGATCCCTCAGACCCTTTTAGTCAGTGTGG-3′. |
| Late RT | MH531 forward 5′-TGTGTGCCCGTCTGTTGTGT-3′, |
| primers, | MH532 reverse 5′-GAGTCCTGCGTCGAGAGAGC-3′, and |
| | LRT probe 5′-CAGTGGCGCCCGAACAGGGA-3′. |
| 2LTR | MH535 forward 5′-AACTAGGGAACCCACTGCTTAAG-3′, |
| circles | MH536 reverse 5′-TCCACAGATCAAGGATATCTTGTC-3′, and |
| | MH603 probe 5′-ACACTACTTGAAGCACTCAAGGCAAGCTTT-3′. |
| Alu-gag | Alu forward 5′-GCC TCC CAA AGT GCT GGG ATT ACA G-3′ |
| primers | HIV gag (Reverse) nucleotides (nt) 1505-1486 5′-GTT CCT GCT |
| | ATG TCA CTT CC-3′ |

### Latency Reversing Agents for HIV-1

One of the most explored therapeutic approaches aimed at eradicating HIV-1 reservoirs is the “shock and kill” strategy which is based on HIV-1 reactivation in latently-infected cells (“shock” phase) while maintaining antiretroviral therapy (ART) in order to prevent spreading of the infection by the neosynthesized virus. This kind of strategy allows for the “kill” phase, during which latently-infected cells die from viral cytopathic effects or from host cytolytic effector mechanisms following viral reactivation. Several latency reversing agents (LRAs) with distinct mechanistic classes have been characterized to reactivate HIV-1 viral gene expression.

Exemplary LRAs are discussed in Ait-Ammar et al. (2020) Frontiers in Microbiology 10:3060, which is incorporated herein by reference. Exemplary LRAs include PMA, JQ1, panobinostat, anti-CD3+anti-CD28, PHA, PMA, prostratin, bryostatin, PMA+ionomycin, TNF-alpha, IL-7+IL-2, SAHA, MRK-1, MRK-11, HMBA, ionomycin, romidepsin, panobinostat, ingenol-3-angelate, Bryostatin-1, IL-15, disulfram, ingenol mebutate, MMQO, JQ1+bryostatin, JQ1+ingenol-B, 5-AzadC+panobinostat, 5-AzadC+romidepsin, chaetocin, and ingenol 3, 20-dibenzoate. Certain classes of LRAs are also shown in Table 7.

TABLE 7
Classes of HIV-1 latency reversing agents

| LRA classes | Examples | Targets |
|---|---|---|
| PKC agonists | Prostratin Bryostatin-1 ingencis: ingenol-B, | NF-κB activation |
| | ingenol 3,20-dibenzoate (ingenol-db), | |
| | ingenol-3-angelate (ingenol mebutate, PEPOOS) | |
| MAPK agonist | Procyanidin trimer C1 | MAP Kinase activation |
| CCR5 antagonist | Miraviroo | NF-κB activation |
| Tat vaccine | Tat Cyl vaccine | Activation of HIV-1 LTR |
| | Tat-R5M4 protein | |
| SMAC mimetics | SBI-0 [symbol] 37142 | Induction of |
| | Birinapant | non-canonical |
| | | NF-κB pathways |
| Inducers of | BETis: JQ1, I-BET, I-BET151, OTX015, | Release of P-TEFb |
| P-TEFb release | UMB-136, MMQO, CPI-203, RVX-208, | |
| | PFI-1, BI-2536 and BI-6727 | |
| Activators of Akt pathway | Disulfram | Upregulation of Akt |
| | | signaling pathway |
| Benzotriazole | 1-hydroxybenzotriazole (HOBt) | STAT5 activation |
| derivatives | | |
| Epigenetic modifiers | HDACis: TSA, trapoxin, SAHA, romidepsin, | HDAC inhibition |
| | panobinostat, entinostat, givinostat, valproic acid | |
| | MRK-1/11, AR-42, fZ,899;nepinostat, chidamide | |
| | HMTis: chaetocin, EPZ-6438, GSK-343, DZNEP, | Suv39H1, G9a, SMYD2 |
| | BIX-01294, UNC-0638 | |
| | DNMTis: 5-AzaC, 5-AzadC | DNMT1, 3a, 3b |
| Immunomodulatory | TLR agonists: TLR2 (Pam3CSK4), TLR7 | |
| LRAs | (GS-9020), TLR8, TLR9 (MGN 1703) agonists | |
| | IL-15 agonist (ALT-803) | |
| | Immune checkpoint inhibitors: anti-PD-1 | |
| | (nivolumab, pembrolizumab), anti-CTLA-4 | |
| | (Ipilimumab) | |

[symbol]

indicates data missing or illegible when filed

### Exemplary Embodiments

1. A composition, comprising
a peptide having at least about 80%, 81%, 82%, 83%, 84%, 85%, 86%, 87%, 88%, 89%, 90%, 91%, 92%, 93%, 94%, 95%, 96%, 97%, 98%, 99%, and/or 100% sequence Leu-Asn); and/or
a peptide having at least about 80%, 81%, 82%, 83%, 84%, 85%, 86%, 87%, 88%, 89%, 90%, 91%, 92%, 94%, 94%, 95%, 96%, 97%, 98%, 99% and/or 100% sequence identity to SEQ ID NO. 1 (lys-Leu-Met-Thr-Pro-Glu-Met-Phe-Ser-Glu-Ile-Leu-Cys-Asp-Leu-Asn) and one or more unnatural amino acids,
or a pharmaceutically acceptable salt, a metabolite, or a carrier thereof.

2. The composition according to 1, wherein the peptide is stapled by forming a covalent linkage between the side-chains of the one or more unnatural amino acids.

3. The composition according to 1 or 2, wherein the one or more unnatural amino acids comprises (S)-2-(4-pentenyl) alanine and/or (R)-2-(7-octenyl) alanine.

4. The composition according to any one of 1-3, wherein two unnatural amino acids are present within the peptide, and wherein the two unnatural amino acids are located within about 1, 2, 3, 4, 5, 6, 7, 8, 9, 10, 11, 13, 14, or 15 amino acid apart from each other.

5. The composition according to any one of 1-4, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-X-Glu-Ile-Leu-X-Cys-Asp-Leu-Asn), wherein X is a non-natural amino acid.

6. The composition according to any one of 1-5, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 2 (lys-Leu-Met-Thr-Pro-Glu-Met-Phe-S5-Glu-Ile-Leu-S5-Cys-Asp-Leu-Asn), wherein S5 is (S)-2-(4-pentenyl) alanine.

7. The composition according to any one of 1-5, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-S5-Glu-Ile-Leu-R8-Cys-Asp-Leu-Asn), wherein wherein R8 is (R)-2-(7-octenyl) alanine and S5 is (S)-2-(4-pentenyl) alanine.

8. The composition according to any one of 1-5, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-R8-Glu-Ile-Leu-S5-Cys-Asp-Leu-Asn), wherein wherein R8 is (R)-2-(7-octenyl) alanine and S5 is (S)-2-(4-pentenyl) alanine.

9. The composition according to any one of 1-4, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-X-Met-Phe-Glu-Ile-Leu-X-Cys-Asp-Leu-Asn), wherein X is a non-natural amino acid.

10. The composition according to any one of 1-4 and 9, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 3 (lys-Leu-Met-Thr-Pro-Glu-R8-Met-Phe-Glu-Ile-Leu-S5-Cys-Asp-Leu-Asn), wherein R8 is (R)-2-(7-octenyl) alanine and S5 is (S)-2-(4-pentenyl) alanine.

11. The composition according to any one of 1-4 and 9, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 3 (lys-Leu-Met-Thr-Pro-Glu-S5-Met-Phe-Glu-Ile-Leu-R8-Cys-Asp-Leu-Asn), wherein R8 is (R)-2-(7-octenyl) alanine and S5 is (S)-2-(4-pentenyl) alanine.

12. The composition according to any one of 1-4 and 9, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 3 (lys-Leu-Met-Thr-Pro-Glu-S5-Met-Phe-Glu-Ile-Leu-S5-Cys-Asp-Leu-Asn), wherein S5 is (S)-2-(4-pentenyl) alanine.

13. The composition according to any one of 1-4, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-X-Glu-Ile-Leu-X-Cys-Gly-Leu-Asn), wherein X is a non-natural amino acid.

14. The composition according to any one of 1-4 and 13, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 4 (lys-Leu-Met-Thr-Pro-Glu-Met-Phe-S5-Glu-Ile-Leu-S5-Cys-Gly-Leu-Asn), wherein S5 is (S)-2-(4-pentenyl) alanine.

15. The composition according to any one of 1-4 and 13, wherein the peptide is stapled and has at least 80%, 81%, 82%, 83%, 84%, 85%, 86%, 87%, 88%, 89%, 90, 91%, 92%, 93%, 94%, 95%, 96%, 97%, 98%, 99%, and/or 100% sequence identity to (lys-Leu-Met-Thr-Pro-Glu-Met-Phe-S5-Glu-Ile-Leu-R8-Cys-Gly-Leu-Asn), wherein R8 is (R)-2-(7-octenyl) alanine and S5 is (S)-2-(4-pentenyl) alanine.

16. The composition according to any one of 1-4 and 13, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-R8-Glu-Ile-Leu-S5-Cys-Gly-Leu-Asn), wherein R8 is (R)-2-(7-octenyl) alanine and S5 is (S)-2-(4-pentenyl) alanine.

17. The composition according to any one of 1-16, wherein the peptide is in an alpha-helical conformation or in a linear conformation.

18. The composition according to any one of 1-17, wherein the peptide disrupts the interaction between human immunodeficiency virus integrase (IN) and INI1/SMARCB1 complex and/or the interaction between IN and trans-activation response element (TAR) RNA.

19. The composition according to any one of 1-18, further comprising at least one antiretroviral agent and/or at least one latency reversing agent (LRA).

20. The composition according to 19, wherein the at least one antiretroviral agent selected from zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, vivecon, and any combination thereof.

21. The composition according to 19, wherein the at least one latency reversing agent is selected from bryostatin-1, ingenol-B, ingenol 3, 20-dibenzoate, ingenol-3-angelate (ingenol mebutate, PEP005), procyanidin trimer C1, maraviroc, tat oyi vaccine, tat-R5M4 protein, SBI-0637142, birinapant, JQ1, I-BET, I-BET151, OTX015, UMB-136, MMQO, CPI-203, RVX-208, PFI-1, BI-2536, BI-6727, HMBA, disulfiram, 1-hydroxybenzotriazol, TSA, trapoxin, SAHA, romidepsin, panobinostat, entinostat, givinostat, valproic acid, MRK-1/11, AR-42, fimepinostat, chidamide, chaetocin, EPZ-6438, GSK-343, DZNEP, BIX-01294, UNC-0638, 5-AzaC, 5-AzadC, Pam3CSK4, GS-9620, MGN1703, ALT-803, anti-PD1 antibody (e.g., nivolumab, pembrolizumab), anti-CTLA4 antibody (e.g., ipilimumab), and any combination thereof.

22. The composition according to any one of 1-21, wherein the composition is a pharmaceutical composition.

23. A method of treating an HIV infection, comprising:
administering to a subject an agent that inhibits or disrupts (i) the interaction between IN and INI1/SMARCB1 complex and/or (ii) the interaction between IN and TAR RNA.

24. The method of 23, wherein the subject is administered with the composition according to any one of 1-22.

25. The method according to 23 or 24, wherein the subject is a human, an animal, a cell, or a tissue.

26. The method according to any one of 23-25, wherein the HIV infection further comprises (i) a condition and/or a disease associated with an HIV-infection, (ii) acquired immunodeficiency syndrome (AIDS), or (iii) a combination thereof.

27. The method according to any one of 23-26, wherein the composition is administered orally, subcutaneously, intramuscularly, or intravenously.

28. A method of reducing a side effect of a therapeutic regime, comprising: administering to a subject the composition according to any one of 1-22, wherein: the subject has received at least one therapeutic regime selected from surgery, antiretroviral therapy (ART), highly active antiretroviral therapy (HAART) or a combination thereof, and the subject is experiencing at least one side effect as a consequence of the therapeutic regime.

29. The method according to 28, wherein the subject has previously been or concurrently being treated with at least one antiretroviral agent selected from zidovudine, didanosine, zalcitabine, stavudine, lamivudine, maraviroc, enfuvirtide, abacavir, emtricitabine, tenofovir, nevirapine, efavirenz, etravirine, rilpivirine, elvitegravir, dolutegravir lopinavir, indinavir, nelfinavir, amprenavir, ritonavir, darunavir, atazanavir, bevirimat, vivecon, and combinations thereof.

30. The method according to 28 or 29, wherein the side effect is selected from drug-resistance, relapse, retention of HIV-infected lymphocytes, generation of a viral reservoir, and combinations thereof.

31. The method according to any one of 28-30, wherein the subject is a human or an animal.

32. The method according to any one of 23-31, wherein the composition or the agent, partially or completely, inhibits the generation of infectious HIV particles, lowers the infectivity of HIV, inhibits spread of HIV, and/or inhibits the infection by the reactivated virus from latent cells.

### EXAMPLES

### Example 1: Materials and Methods for Examples 2-8

Cloning of INI1183-265, INI1183-304, and IN fragments. Fragments containing INI1183-265 and INI1183-304 were cloned into pET28a-h6-smt3 vector using sequence and ligation-independent cloning (SLIC) method54. Briefly, vector sequences were PCR-amplified using the primers, VFOR and VREV (Table 6a). INI1₁₈₃₋₂₆₅ and INI1₁₈₃₋₃₀₄ fragments were amplified using the forward primer S6(Rpt1)-For and two different reverse primers, INI1(aa 265)-Rev and INI1(aa 304)-Rev, respectively (Table 6a). PCR was performed using Phusion Polymerase followed by digestion of PCR amplified fragment with DpnI for 2-4 h or overnight at 37° C., purified using Qiagen PCR purification kit (Catalogue #28104) and then gel purified (Catalogue #28704). The gel-purified fragments were subjected to T4 DNA polymerase reaction to generate single stranded overhangs in the absence of dNTPs at room temperature for 30 m followed by quenching the reaction by the addition of 0.5 mM dNTP and immediately heating at 65° C. for 10 m to deactivate the T4 enzyme. The SLIC reaction is set up by mixing 1:3 molar ratio of vector to insert, 10× ligation buffer, and 20 mM ATP and incubated at 37° C. for 30 m. A total of 1-2 μl of the reaction mixture was then transformed into E. coli. The resulting transformants were sequenced to confirm the presence of INI1 insert using T4 terminator primer (Novagen Catalogue #69337 5 pmole/μl).

To clone GST-IN, GST-NTD, GST-CCD, and GST-CTD, fragments encoding the three IN domains were PCR amplified using the HIV-1Hx3B DNA as a template, and were inserted into pGEX3xPL vector to obtain the respective GST-fusion expression constructs.

Expression and purification of INI1₁₈₃₋₂₆₅. Expression of His6⁻SUMO⁻INI¹183-265 protein from the plasmid (pET28a-h6-smt3-INI1183-265) in E. coli was confirmed by immunoblot analysis using α-6His [Clontech, Catalogue #631212; Lot #8071803; 1:1000 dilution) and α-BAF47 (BD Transduction laboratories, Catalogue #612110; Lot #7144795; 1:1000 dilution) antibodies. E. coli strain BL21(DE3)lysS harboring the expression plasmid was induced with 1 mM IPTG, resuspended in lysis buffer (25 mM HEPES, pH 7.4, 10% glycerol, 1 mM PMSF and 0.1% Triton X-100), and subjected to sonication. The sonicated culture was rocked for 45 m, clarified by centrifugation and was loaded on to pre-equilibrated Ni-NTA column in buffer (1 mM HEPES, pH 7.4, 10% glycerol, 0.5 M NaCl and 30 mM Imidazole). The bound proteins were washed with several volumes of the same buffer and eluted in 25 mM HEPES, pH 7.5, 10% glycerol, 0.5 M NaCl, and 300 mM imidazole. The eluted protein was digested with SUMO protease for ˜16+h at 4° C. with rocking. After proteolysis, the buffer was exchanged with holding buffer (25 mM HEPES, pH 7.4, 10% glycerol, and 0.25 M NaCl), by spinning with Amicon Ultra-15 Centrifugal Filter unit. The His6-SUMO tag was removed by running protein on Ni-NTA column equilibrated with holding buffer. Finally, the eluted and purified protein was passed through 16/60 Superdex 200 gel filtration column, using the buffer, 25 mM HEPES, pH 7.4, 0.25 M NaCl, 2 mM DTT, and 5% glycerol. The protein eluted as a single peak and was collected and dialyzed in the buffer 25 mM HEPES, pH 7.4, 5% glycerol, 0.15 M NaCl, 1 mM EDTA, and 2 mM DTT.

To purify the labeled INI1₁₈₃₋₂₆₅ for NMR studies, Rosetta (DE3) cells (Novagen) were transformed with pET28a-h6-smt3-INI1₁₈₃₋₂₆₅ (clone 3.1) expression plasmid and the cultures were grown at 37° C. in 1 L of minimal medium supplemented with 1 g ¹⁵NH4Cl and 2 g ¹³C-glucose (Cambridge Isotope Laboratories). The bacteria were induced at a cell density of _{OD600} 0.6 by the addition of 0.5 mM IPTG and were then incubated at 22° C. overnight. The cells were pelleted by centrifugation at 7,000 g for 15 minutes and the pellets were stored at −80° C. for further processing. The pellets were thawed and resuspended in lysis buffer [20 mM HEPES pH 7.6, 500 mM NaCl, 20 mM imidazole, 10% glycerol containing 0.2 mM 4-(2-aminoethyl) benzene sulfonyl fluoride hydrochloride (AEBSF) and 4 units/ml of DNase I]. The cells were lysed using an Emulsiflex C3 (Avestin) and the lysate was centrifuged at 17,000 g for 1 hr at 4° C. The supernatant was applied to a prepacked His60 superflow column (Clontech) using an ÄKTA avant purification system (GE Healthcare). The column washed with 10 column volumes (CV) of lysis buffer without protease inhibitor and DNase. The protein was eluted with 20 mM HEPES pH 7.6, 500 mM NaCl, 500 mM imidazole, 10% glycerol, and then loaded onto a Superdex 200 16/60 column (GE Healthcare) in SEC buffer (20 mM HEPES pH 7.6, 150 mM NaCl, 5% glycerol and 10 mM DTT). The fractions containing the protein of interest were pooled together and digested with SUMO hydrolase (ratio 100 to 1 respectively) overnight at 4° C. The his-SMt3 tag was removed by loading the digested proteins onto a prepacked Ni Sepharose high performance column equilibrated in 20 mM HEPES pH 7.6, 150 mM NaCl, 5% glycerol, and 10 mM DTT. The column was washed with 3CV of SEC buffer. The flow-through and the washes containing the protein of interest were pooled together and concentrated using a Vivaspin 20 filter with a 3 kDa cutoff (Sartorius AG). The protein was finally loaded into a Superdex75 16/90 column (GE Healthcare) in NMR buffer (10 mM sodium phosphate pH 6.8, 150 mM NaCl, 1 mM EDTA, 5 mM TCEP). The fractions containing the protein were concentrated using a Vivaspin 20 filter with a 3 kDa cut-off (Sartorius AG) and snap frozen for storage at −80° C. SDS-PAGE was used to determine the sample purity and the correct identity of the purified protein was achieved by LC-MS/MS.

Nuclear magnetic resonance. Isotopically enriched INI1183-265 was purified and concentrated to 1 mM in 150 mM NaCl, 10 mM sodium phosphate, 10% D2O, and 5 mM BME (pH 7.4). All NMR data were acquired at 25° C. on a 600 MHz cryoprobe-equipped Agilent instrument, or at 900 MHz on a Bruker Avance II. NMR data was collected using manufacturer's TopSpin v2.3 operating software.

Sequence specific and side-chain assignments were obtained by standard nD triple resonance methods. All 3D experiments were acquired as non-uniform sampled experiments using the MDDNMR v2.1 approach55. All NMR data sets were processed with nmrPipe/nmrDraw56 and analyzed using CCPN Analysis 2.357. Chemical shifts were indirectly referenced to 4,4-dimethyl-4-silapentane-1-sulfonic acid (DSS). Interproton distance restraints were derived from 3D 15N- and 13C-edited NOESY-HSQC spectra with a mixing time of 150 ms. The full table of characterization is reported.

Analytical ultracentrifugation. Sedimentation velocity analyses were conducted in a Beckman XL-I analytical ultracentrifuge at 270,789×g and 25° C. using double sector centerpieces in the An60 rotor. Sedimentation was tracked using the absorption optics at 280 nm. The buffer was 20 mM HEPES pH 7.5, 300 mM NaCl, 2 mM DTT, and 5% glycerol. The reported sedimentation parameters were cor-rected to standard conditions (20, w) using values of the partial specific volume=0.742, solvent density=1.02 g/mL, and solvent viscosity=1.16 cp. The data were analyzed using the time-derivative method implemented in the program DCDT+58,59. The best fit values and 68.3% confidence limits are reported.

Protein structure modeling and analysis. Robetta was used to builds various models for Rpt1-Rpt2 and 183-304 fragments of INI1. Robetta generates three- and nine-residue fragment libraries that represent local conformations seen in the PDB, and then assembles models by fragment insertion to form low-energy global structures. Detailed methodology of Robetta can be accessed from²⁵ and robetta.bakerlab.org. To assess the overall stereochemical quality of the generated 3D model, the geometrical accuracy of the residues and 3D profile quality index were inspected with the PROCHECK (ver. 3.5)²⁶. Additionally, WHATIF (ver. 8.0) modeling package software was used to analyze the quality of model by checking clashes⁶⁰. The modeled protein is also validated by VERIFY3D, which checks compatibility of 3D models with its sequences²⁸. The statistics of non-bonded interactions between different atom types were detected and value of the error function was analyzed by ERRAT⁶¹. PROSA was used for final model to check energy criteria²⁹.

Molecular docking studies. First, the INI1₁₈₃₋₃₀₄ fragment and CTD were docked using HADDOCK v2.4⁶². The docking was done ab initio, without providing interaction restraints; instead center-of-mass restraints were provided to guide the docking. The center-of-mass restraints are automatically generated by calculating the dimensions of each molecule along the x, y and z axes (dₓ, d_{y}, d_{z}) and summing the average of the two smallest components per molecule. The resulting distance was used to define a restraint between the center of mass of each subunit with an additional upper bound correction of 1 Å. Finally, clustering was done to sort the docking solutions. The center of the best cluster was taken as the final model and was analyzed further in detail.

Second, both INI1₁₈₃₋₃₀₄ fragment and TAR RNA were docked separately onto IN-CTD using MDockPP⁴³. The docking process was performed by heavily sampling the relative binding orientations, creating 54,000 putative binding poses. These binding poses were screened by the following experimental constraints: The residues W235, R228, K264, K266, and R269 of CTD were required to be within 5 Å of the INI1₁₈₃₋₃₀₄ fragment and TAR RNA, respectively. For the INI1-Rpt1/IN-CTD structure prediction, an extra constraint was imposed that D225 of INI1-Rpt1 was within 5 A of IN-CTD. The surviving poses were then rescored with ITScorePP^{44,45}, and the top-ranking pose was selected and optimized using UCSF Chimera⁶³ (www.cgl.ucsf.edu/chimera). Structure visualization, structure characterization and analysis, and image rendering were carried out using CCP4 mg (www.ccp4.ac.uk/MG/), MacPymol (PyMOL v1.7.6.4 Enhanced for Mac OS X, pymol.org/), Maestro (Schrodinger, www.schrodinger.com/maestro), and UCSF Chimera (version 1.14 at www.cgl.ucsf.edu/chimera).

Generation of substitution mutations. Mutagenesis was carried out using QuickChange Lightning site-directed mutagenesis kit (Agilent Catalog #210518).

For introducing mutations into INI1183-304 fragment, pET28a-h6-smt3-INI1183-304 plasmid was used as template. Primers used for mutagenesis are provided in Table 6b. For introducing mutations into the CTD domain of IN, the plasmids pGEX3x-IN and pGST-IN-CTD(aa 201-288) were used as templates and the primers used for mutagenesis are provided in Table 6c.

The mutant clones were sequenced using the T7 terminator primers for pET clones and GST-forward and reverse primer for GST clones to confirm the presence of mutations in the sequence.

Generation of W235, R228A, and K244A mutant viruses. HIV-1_{NL4-3} viral clones containing IN mutations were generated in two stages. First, a 2.3 Kb Age I to Sal I (position 3485 bp to 5785 bp) fragment of _{HIV-1NL4-3} containing the IN open reading frame was subcloned into the pEGFPN1 vector to generate an intermediate vector pEGFPN1-IN-int. IN mutations were introduced into pEGFPN1-IN-int using the QuikChange II Mutagenesis Kit (Stratagene). The Sal I/Age I fragment containing the desired mutation was subsequently cloned into pNL4-3 and in some cases into pNL4-3.Luc.R-E-[HIV-Luc] to obtain mutant viral clones for use in multiday and single cycle infection assays.

GST pull-down assays. 5 ug of GST-IN or GST-CTD and their mutant proteins bound to glutathione sepharose 4B beads were incubated for 1 hour at 4° C. with normalized amounts of clarified bacterial cells lysates containing His6-SUMO-INI¹183-304 WT and its mutants in binding buffer (20 mM HEPES—pH 8.0, 5 mM DTT, 0.5% IGEPAL, 200 mM NaCl, and protease inhibitor tablet Roche Catalogue #11836170001). Following incubation, beads were washed 5-7 times with buffer containing 50 mM Tris-Cl pH 8.0, 1 mM EDTA, 500 mM NaCl, 0.5% IGEPAL, 25 mM PMSF. Bound proteins were separated by SDS-PAGE and analyzed using α-BAF47 antibody to detect bound INI1 or 6His-SUMO-INI1₁₈₃₋₃₀₄ proteins.

AlphaScreen proximity assay to detect the protein-protein and protein-RNA interactions. AlphaScreen assay was carried out using PerkinElmer Alpha-Lisa-6His Acceptor beads (Catalogue #AL128C), Alpha-Screen-GST donor beads (Catalogue #6765300), GST-tagged and His-tagged proteins for protein-protein interactions; and Alpha-Screen Streptavidin donor beads (Catalogue #6760002 S), Alpha-Lisa-GST acceptor beads (Catalogue #AL110C), Biotin-labeled RNA and GST-tagged proteins for protein-RNA interactions. The reaction was carried out according to the manufacturer's protocol in a reaction buffer contacting (25 mM HEPES pH 7.4, 100 mM NaCl (or 500 mM NaCl), 1 mM DTT, 1 mM MgCl2 and BSA 1 mg ml/ml). The mixture of proteins or protein and RNA were incubated for 1 hr at room temperate with shaking in a multi micro-plate shaker and then 20 ag ml/ml accepter beads were added and incubated for additional 1 h with shaking. Finally, 20 ag ml⁻¹ of donor beads were added and further incubated for 1 hr. The Alpha score readings were measured using Alpha-compatible Envision 2105 multi-plate reader (Perkin-Elmer). All the experiments related to this method were carried out 3 to 5 times and data were analyzed by using Graph Pad Prism 9 version 9.0.0 (GraphPad Software, CA). The RNA sequences used in this assay are listed in the Table 6d and were obtained from Integrated DNA technology.

Co-IP and RNA-co-IP to determine interaction of IN, INI1, and TAR RNA in vivo. For co-IP studies, pYFP-IN or mutants and pCGN-INI1 (expressing HA-INI1) were transfected into MON(INI1−/−) cells. The transfected cells were lysed in 20 mM Tris-HCL (pH 8), 150 mM NaCl, 1% Triton X 100, 2 mM EDTA, 40 μL/ml Protease cocktail inhibitor (Roche cat no: 11836170001) and 40 μL/ml RNAse inhibitor (Invitrogen Cat. No: 10777019). The lysates were pre-immunoprecipitated with isotype IgG antibodies (Santa Cruise Biotechnology, Catalogue #SC-51993; Lot #F0316, 5 ag/sample) and then immunoprecipitated using α-HA (Santa Cruz, Catalogue #SC-7392; Lot #L1218; 5 μg/sample) antibodies overnight with shaking at 4 C. The immunocomplex was subjected to Western blot analysis using either a-IN (NIH AIDS reagent and repository, Catalogue #3514; Lot #130353, 1:500 dilution) or a-BAF47 antibodies.

For RNA-co-IP, MON cells were transfected with pCMV-Tat and pLTR-luc plasmids along with pYFP-IN and pCGN-INI1. Immunopreciptation was carried out as above using α-GFP (Cell Signal, Catalogue #2555; Lot #8; 5 ag/sample) antibodies. The immunecomplexes were then split into two and RNA was isolated from one half using Trizol reagent (Invitrogen Catalogue #15596026) and subjected to qRT-PCR using Early RT primers. The other half was subjected to immunoblot analysis.

As a negative control for IP and RNA-co-IP, isotype specific IgG (Santa Cruise Biotechnology, Catalogue #SC-51993; Lot #F0316, 5n/sample) antibodies were used.

Virus production and multiday infection. _{HIV-1NL4-3} viral stocks of wild type, W235E and R228A mutants were prepared by transient transfection of 30-40% confluent 293 T cells in 10 cm² plates with 10 μg viral DNA. Virus was collected 48 h post-transfection and clarified by passing through a 0.45 μm cellulose acetate filter (Corning). Clarified supernatant was then treated for 30 m with 20 units/mL of DNase I (Roche Catalogue #04716788001) at 37° C. For the purpose of CryoET, the virions were concentrated using sucrose step gradient⁶⁴. Viral stocks were measured for p24 using a p24 enzyme-linked immunosorbent assay (ELISA) (Advanced Bioscience Laboratories).

For the purpose of multiday infections, 25 ng p24 of each _{HIV-1NL4-3} wild type or W235E mutant viruses were incubated with 200,000 CEM-GFP cells for 2 h in a 2 mL culture. After incubation with virus, 18 mL of complete RPMI1640 was added to the culture in a 75 cm² flask and incubated at 37° C. for several days. 1 mL of culture was collected every two days for 16 days. Viral replication was monitored by observing cellular GFP levels and by measuring p24 levels in the culture supernatant.

Real-time PCR to detect early and late RT products, 2LTR circles, and integrated product. A total of 50% confluent 293 T cells were spinoculated with 20 ng p24 of HIV-Luc virus for 2 h at 15° C. and spun at 400 g. Cells were collected at various time points post-infection and harvested for genomic DNA using the DNeasy Blood and Tissue Kit (QIAGEN). About 300-500 ng genomic DNA was used per 50 aL real-time PCR reaction containing 300 nM primers, 100 nM probe and 2× Tagman Universal Master Mix (Applied Biosystems). The primers and probes that were used to detect early and late RT products⁶⁵ are listed in the Table 6e. Cycling conditions on an ABI 7900HT are 2 m at 50° C., 10 m at 95° C., followed by 40 cycles of 15 s at 95° C. and 1 m at 60° C.

To detect the integrated provirus, nested Alu-PCR was performed⁶⁶. The genomic DNA was isolated 24 hours post-infection. In the first semi-quantitative PCR round, Alu-gag was amplified using the primers listed in Table 6e. The Alu-gag PCR product was then subjected to a second round of qPCR using the MH531, MH532, and late RT primers and probe (Table 6e) using conditions described above. Standard curves were generated by isolating genomic DNA from 293 T cells stably infected with HIV-1 containing GFP and a hygromycin-resistance marker⁶⁶ and by performing nested PCR from this cell line. Ct values were then plotted against dilutions of the hygromycin-resistant HIV-1 plasmid to create a standard curve.

Electron microscopy. Monolayer 293 T cell cultures producing _{HIV-1NL4-3} WT or R228A mutant were fixed with 2.5% glutaraldehyde, in 0.1 M sodium cacodylate buffer, post-fixed with 1% osmium tetroxide followed by 2% uranyl acetate, dehydrated through a graded series of ethanol, cells lifted from the monolayer with propylene oxide and embedded as a loose pellet in LX112 resin (LADD Research Industries, Burlington VT) in eppendorf tubes. Ultrathin sections were cut on a LeicaUltracut UC7 (Leica Microsystems Inc., Buffalo Grove, IL), stained with uranyl acetate followed by lead citrate and viewed on a JEOL JEM 1400Plus transmission electron microscope (JEOL USA, Peabody, MA) at 120 kV.

Cryo-ET. Grids of HIV-1 WT and W235E virions were prepared as follows⁶⁷. Purified fixed virus was mixed (2:1) with a suspension of colloidal gold particles (Electron Microscopy Sciences), applied to glow-discharged Quantifoil R2/2 200-mesh holey carbon grids (Structure Probe, Inc.), blotted, and plunge-frozen using a Leica EM GP (Leica Microsystems). For data acquisition, grids were transferred to a cryo-holder (type 914; Gatan), and single-axis tilt series were recorded at 200 keV on a JEOL 2200FS electron microscope equipped with an in-column energy filter (20 eV energy slit). Images were acquired on a K2 Summit direct electron detector (Gatan) in super-resolution mode at nominal 10 K magnification, giving a super-resolution sampling rate of 1.83 Å/pixel. Using SerialEM⁶⁸, dose-fractionated projections were typically acquired at 2° intervals from −56° to +56° with a target defocus of −2.5 am. The electron dose per exposure (five 0.2 s frames each) was −1.3 e⁻/Å², giving a total cumulative dose of −75 e⁻/Å².

For Image Processing and Analysis, Unbinned frames were aligned and averaged using the bseries function in Bsoft⁶⁹, and the motion-corrected tilt-series images were binned by four. Tomograms were reconstructed using Bsoft, and virions were extracted and denoised by 20 iterations of anisotropic nonlinear diffusion⁷⁰. Denoised particles were then manually inspected for defects in assembly.

Statistics and reproducibility. Data are presented as mean value ±SEM (standard error of mean), calculated using GraphPad Prism 9 version 9.0.0. All experiments were conducted a minimum of three independent times using three independent preparations of the same protein or virus and were found to be reproducible. N values are indicated within figure legends and refer to biological replicates (independent experiments conducted using different preparations of proteins or viruses).c

### Example 2: The Three Dimensional Structure of the Complex Between IN-INI1 Domains were Determined Using the NMR Structures, and the Interacting Residues were Identified

This interaction provides the first clues about the interface residues involved in interaction. Details of this interaction are part of present disclosure supra.

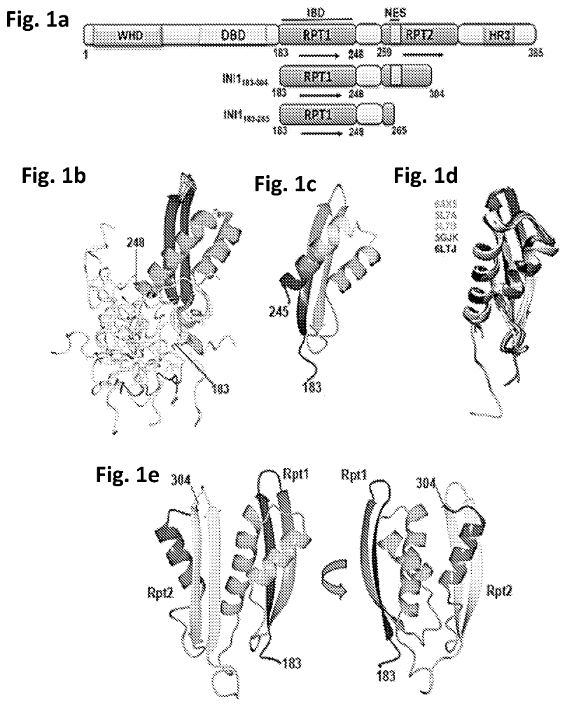

As disclosed herein, the NMR structure of INI1183-265 PDB ID: 6AX5 (http://doi.org/10.2210/pdb6AX5/pdb) and FIG. 1a-c] was solved. While Rpt1(INI1183-245) is sufficient for IN binding¹², a longer fragment S6(INI1183-294) harboring Rpt1+linker+partRpt2, shows stronger binding and acts as a dominant negative inhibitor of HIV-1¹⁴. To determine the contribution of INI1-Rpt2 for IN binding, we modelled INI1183-319 fragment containing Rpt1-linker-Rpt2 based on 6AX5 structure using Robetta¹⁶ (FIG. 1e). This modeled structure was validated using various tools including Ramachandran plot, PROCHECK version 3.5¹⁷ WHATIF ver. 8.0¹⁸, VERIFY3D v3.1¹⁹ and PROSA 2003²⁰, which strongly supported the features of the model (data not shown).

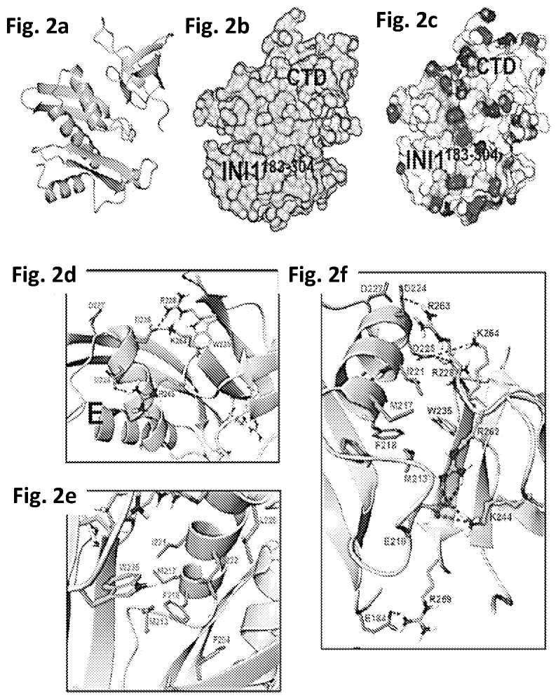

We computationally docked the INI1₁₈₃₋₃₀₄ structure with the NMR structure of IN-CTD [1QMC (http://doi.org/10.2210/pdb1QMC/pdb)]^{21,22} using HADDOCK, without interaction restraints (FIG. 2a-c). In the docked complex, the exposed negatively charged Rpt1 residues of INI1183-304 contacted the basic residues of IN-CTD, and the interaction of hydrophobic residues of the two proteins resulted in a hydrophobic core within the complex (FIG. 2d,e and f). In the model of the complex, acidic residues D225 and D224 from a1 of INTI1-Rpt1 made polar contacts with basic IN-CTD residues R228 (in strand 31), K264, and R263 (in the loop between 34 and 35 strands) (FIG. 2d). Additional ionic interactions were noted between INI1183-304 E210 (in the loop region between 32 and a1) and IN-CTD residues R262 (in the loop between 34 and 35 strands) and K244 (in the loop between 32 and 33 strands) (FIG. 2f). The TN-CTD W235 residue formed the center of the Rpt1/CTD interface, surrounded by a shallow hydrophobic patch/cage formed by F204, L226, L222, I221 and F228 residues from a1 of Rpt1 (FIGS. 2e and f). This model was validated by biochemical studies of interface residue mutants. Substitution mutations of multiple interface residues in IN and INI1 confirmed the model (data not shown).

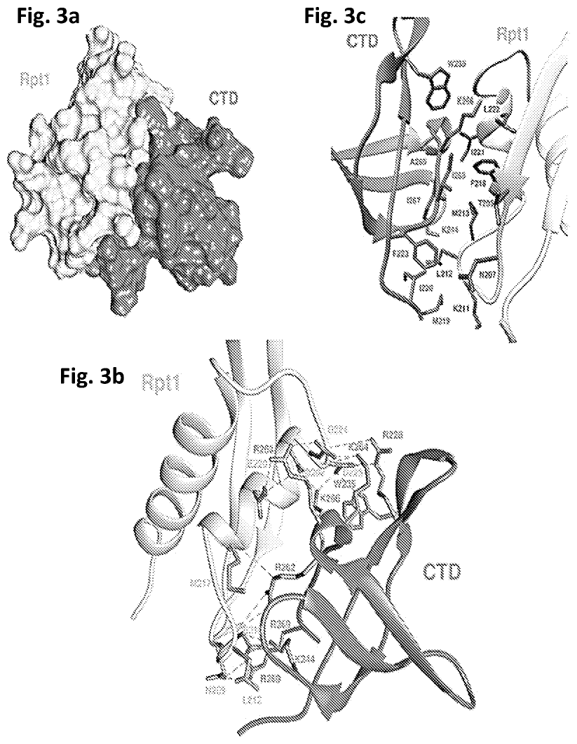

We further refined the IN-CTD/INI1183-304 complex model by providing interaction restraints for the interface residues based on experimental data, using the in-house docking software, MDockPP²³, (FIG. 3). The docked complex with the lowest (best) score of ITScorePP^{24,25}, indicated that upon complex formation ˜865.0 Å² of the solvent-accessible surface. We identified a total of 14 hydrogen bonds formed across the interface (FIG. 3b). The 6 residues on IN-CTD (R228, W235, K244, R262, R263 and K264) and 6 residues on INI1-Rpt1 (E210, L212, M217, E220, D224 and D225) constituted a strip of hydrogen-bond network, establishing binding specificity (FIG. 3b). In addition, the binding affinity was conferred by hydrophobic interactions between INI1-Rpt1 and IN-CTD (FIG. 3c). On IN-CTD, a patch of the van der Waal surface that consists of a group of mostly hydrophobic residues (I220, F223, W235, A265, 267 and I268) matched the shape of a patch on Rpt1 that was also defined by mostly hydrophobic residues (L212, M213, F218, I221 and L222) (FIG. 3c). The region of the hydrophobic interactions was encircled by the residues forming the hydrogen-bonding network.

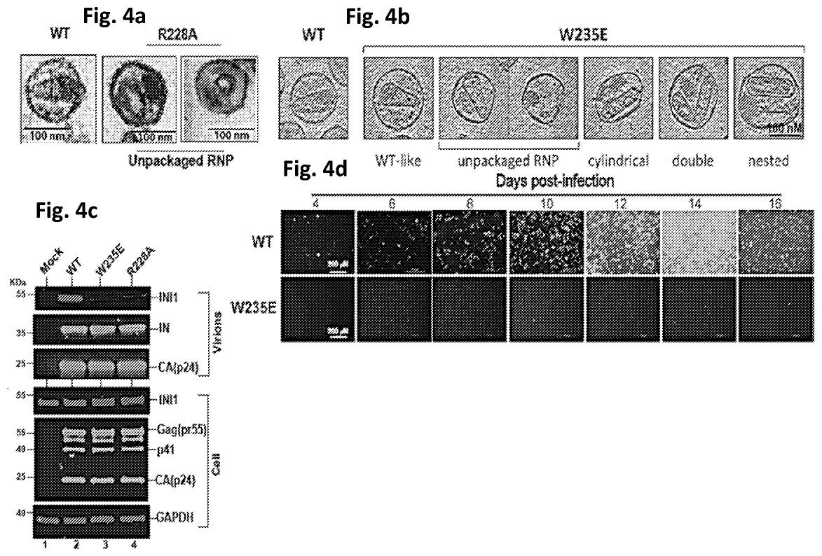

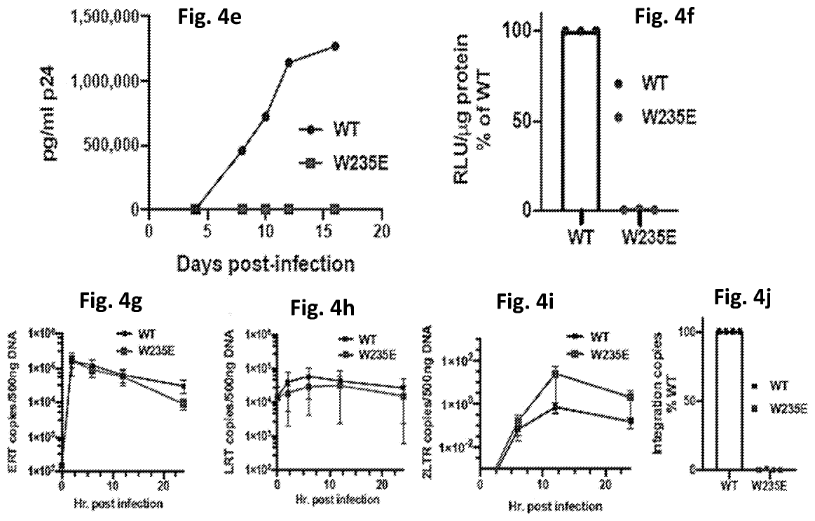

### Example 3: Biochemical and Virological Analyses Validated the IN-CTD/INI1183-304 Model and Demonstrate that IN-INI1 Interactions are Necessary for HIV-1 Replication (FIG. 4)

This was demonstrated by mutating the interface residues of IN. We found that mutating IN-interface residues led to: i) Disruption of IN-INI1 interaction in vitro and in vivo; ii) led to formation of morphologically defective particles by electron microscopy (FIGS. 4a and b); iii) inhibited the incorporation of INI1 into virions (FIG. 4c); and iv) produced virions which were defective for infection and integration (FIG. 4d-j). These studies established the functional significance of the interface residues.

The above studies also indicated that the interface IN residues overlapped with those IN residues important for its interaction with HIV-1 genomic RNA, specifically in the TAR region (data not shown). This hypothesis was confirmed by demonstrating that: i) IN mutants showed the same pattern of interaction with INI1 and TAR RNA; ii) INI1-Rpt1 and TAR RNA competed with each other for binding to IN in vitro and in vivo; and iii) Both INI1-interaction defective and RNA-interaction-defective mutants of IN led to production of non-infectious morphologically defective particles. These studies led us to hypothesize that TAR RNA and INI1183-304 binds to the same surface and residues of IN. To determine the structural basis of this similarity in binding we modeled the binding of IN-CTD to TAR RNA, which was not known before.

### Example 4: IN-CTD Interaction with TAR RNA Using the NMR Structures

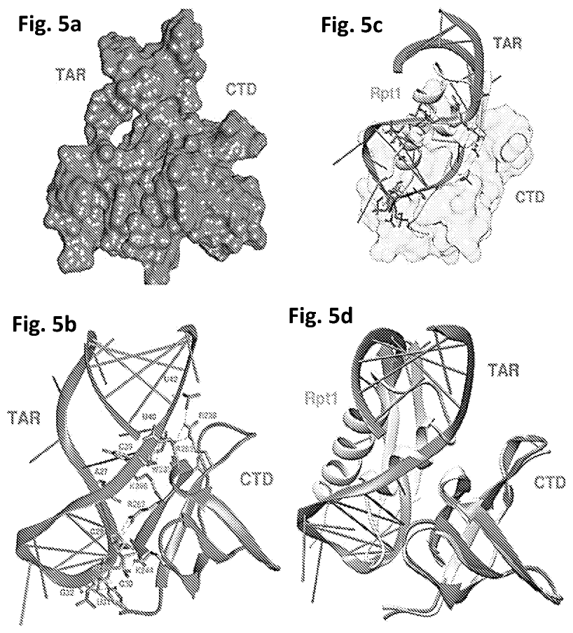

To understand the similarity in interaction of INI1-Rpt1 and TAR RNA with IN-CTD, we generated a docking model of IN-CTD/TAR RNA complex by using the lowest energy conformer from the NMR structures of TAR [1ANR (http://doi.org/10.2210/pdb1ANR/pdb)] and that of IN-CTD [1QMC (http://doi.org/10.2210/pdb1QMC/pdb)] and by using MDockPP²³. After applying the experimental restraints, the top docked structure by reranking with ITScorePR²⁵ showed that the TAR RNA forms a complex with IN-CTD through the same interface region of IN-CTD as in the IN-CTD/INI1183-304 complex, covering a similar net surface area of 826 Å² (FIG. 5a). This model indicated that the arginine and lysine residues of IN-CTD make multiple hydrogen bonds and electrostatic interactions with the phosphate backbone of TAR RNA (nucleotides 27-32 and 37-42) constituting the binding interface. IN-CTD specifically bound the minor groove of TAR RNA through 14 hydrogen bonds (similar to that of INI1-Rpt1) across the interface, including R228-U40, R228-U42, R262-C29, R263-C39, R269-U31, R269-G32, K244-C30, K266-A27 and K266-C39 (FIGS. 5b and c). Meanwhile, the hydrophobic residues at the interface of IN-CTD protruded into the minor groove to form non-polar interaction with the hydrophobic part of the bases (FIG. 5b).

### Example 5: INI1-Rpt1 Domain is an RNA Mimic and that INI1-Rpt1 and TAR RNA Structurally Mimic Each Other (FIG. 5)

When IN-CTD/TAR and IN-CTD/Rpt1 complexes w ere structurally aligned over IN-CTD, it became evident that both TAR and INI1-Rpt1 engage with the same binding interface and same residues of IN-CTD (FIG. 5d). There were only minor conformational differences in the side-chains in the IN-CTD interface residues, suggesting that no large side-chain rearrangements were required when switching from INI1-Rpt1 to TAR. The structural elements of INI1-Rpt1 and backbones of TAR seemed to perfectly line up in space (FIG. 5d). Moreover, the phosphate groups of TAR RNA were present in close proximity with the negatively charged residues of INI1-Rpt1 when the two docked complexes were superimposed (5c). In addition, similar buried solvent accessible surface areas, 865 Å² for IN-CTD/Rpt1 versus 826 Å² for IN-CTD/TAR, further supported that the two binding sites were the same for Rpt1 and TAR.

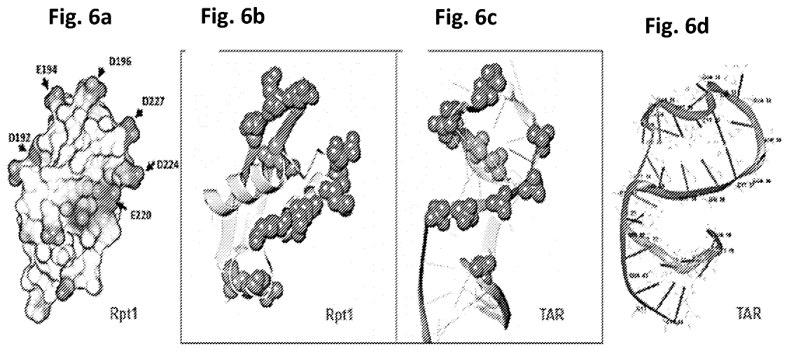

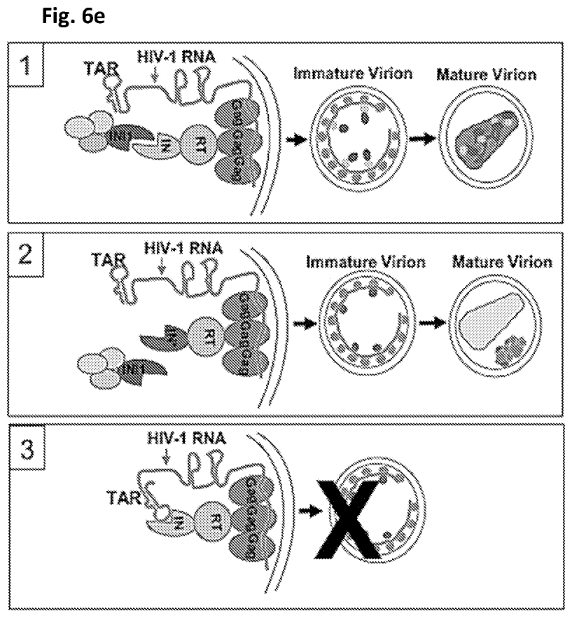

As presented herein, in the INI1-Rpt1 structure, the two β-sheets and helices of the INI1-Rpt1 (aa 183-248) came together to form a central hydrophobic barrel like core, which was decorated by a string of negatively charged surface exposed residues D192, E194, D196, E220, D224, D225 and D227 (FIG. 6a). Comparison of the arrangement of negatively charged residues of Rpt1 to the phosphate groups on the TAR RNA NMR structure (FIG. 6d), demonstrated a similar placement of negative charges on the two molecules (FIGS. 6b and c). This observation, combined with the observation of the close proximity of the charged residues of INI1-Rpt1 with the phosphate residues of the TAR RNA when the two complexes were superimposed (FIG. 5c), suggested that the INI1-Rpt1 domain structurally mimic TAR RNA involved in binding to IN-CTD (FIGS. 6b and c).

### Example 6: Stapled Peptide Inhibitors were have Synthesized Based in the Alpha Helix in the INI1-Rpt1 Structure that Forms the Interface Between INI1 and IN and have Used this Stapled Peptide Drug to Inhibit HIV-1 Replication

Structural mimicry between INI1-Rpt1 and TAR RNA, their similar interaction with IN, and the requirement of this interaction for production of infectious virions suggested that drugs that target IN-INI1 interaction could dually inhibit both IN-INI1 and IN-RNA interactions and that these drugs could inhibit late events of HIV-1 replication.

Stapled peptides Derived from INI1 Alpha 1 Helix Disrupt IN/RNA and IN/INI1 Interactions

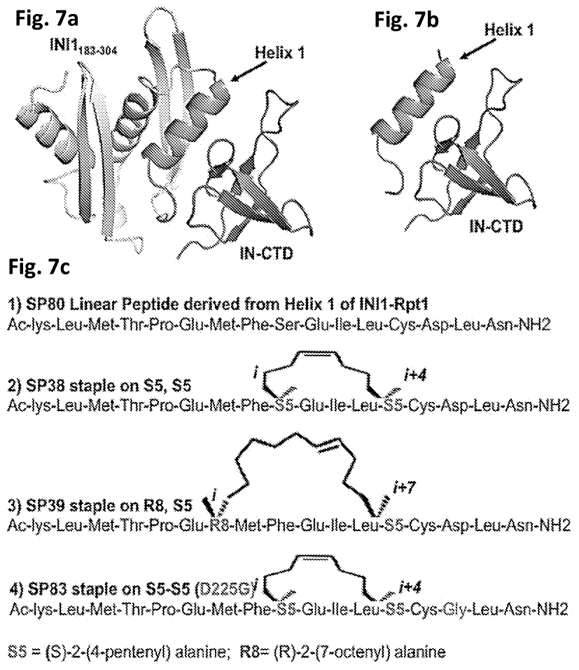

As a proof of concept, we chemically synthesized stapled peptides derived from interface α1helix region of INI1. This helix overlaps with the TAR RNA backbone that is at the interface interacting with TN-CTD, based on our model (FIG. 5d and FIGS. 7a and b). Four peptides were synthesized (FIG. 7c): i) Linear peptide (SP80); ii) Stapled peptide, stapled at i, i+4 residues (SP38); iii) Stapled peptide, stapled at i, +7 residues (SP39); and iv) mutant stapled peptide SP83, which is same as SP38 but mutated at the interface residue D225G, which has been shown to disrupt INI1 interaction with IN.

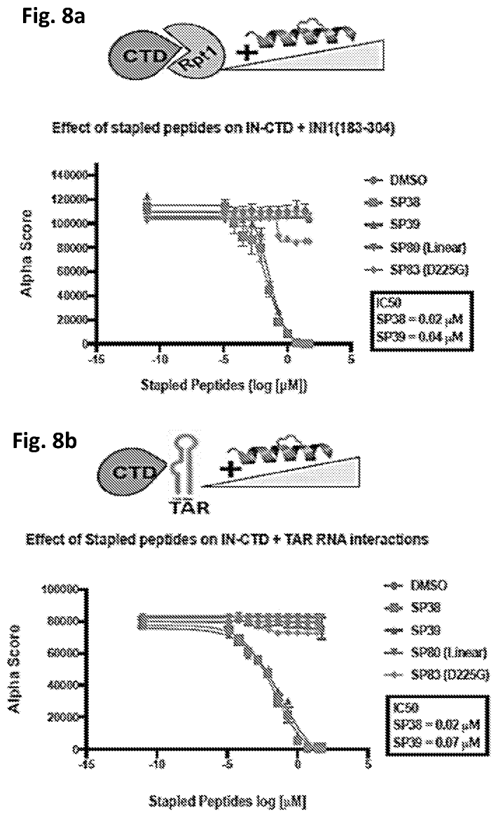

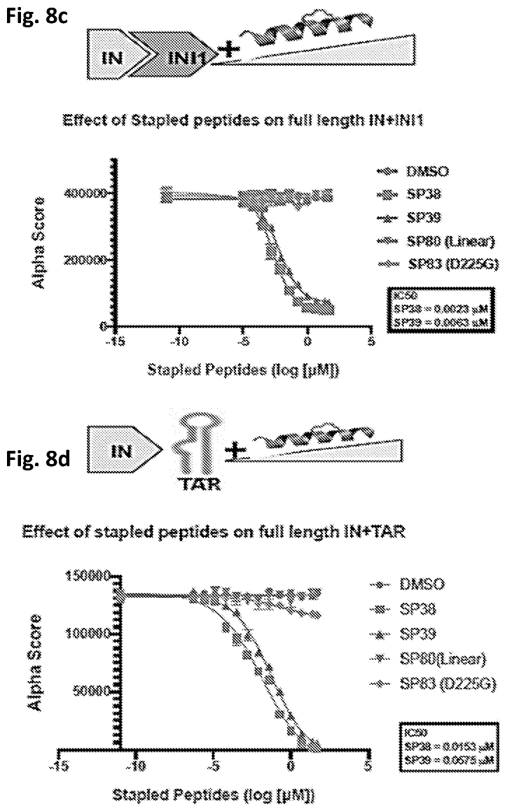

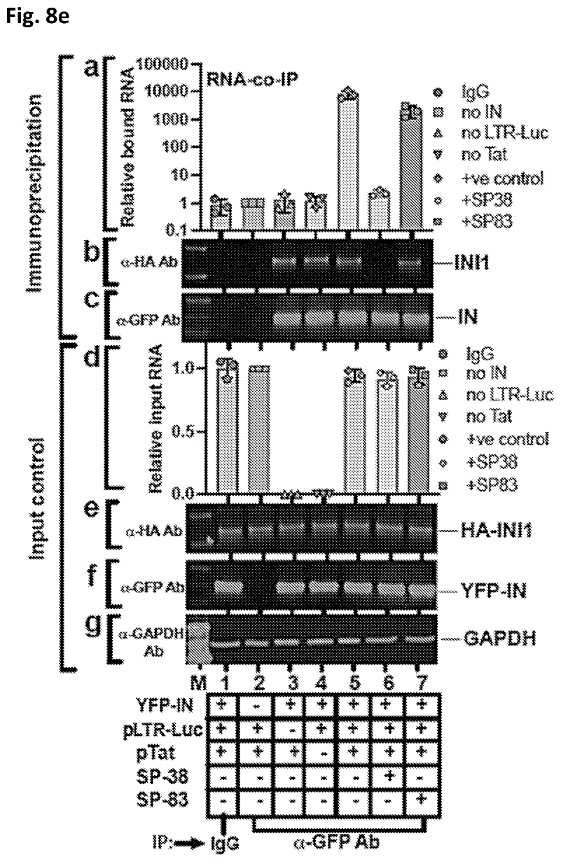

These peptides were tested for their ability to disrupt the interaction between IN-INI1 and IN-TAR RNA in vitro. As expected, linear peptide did not inhibit IN-INI1 or IN-TAR RNA interactions (FIG. 8). But stapled peptides (SP38 and SP39) but not the mutant peptide SP38 potently inhibited the IN-INI1 or IN-TAR RNA interactions with an IC50 at low nanomolar concentrations (FIG. 8).

### Stapled Peptides Inhibit IN/RNA Interactions in Cells

To determine if stapled peptides can inhibit interaction of IN with TAR RNA in cells, we carried out RNA-co-IP experiments (FIG. 8e). YFP-IN and TAR RNA were expressed by co-transfecting plasmids pYFP-fN, pLTIR-luc and pTat in 293T cells. Cells were treated with SP-38 or SP-83 (D225G mutant) peptides. After transfection, the cell lysates were treated with DNaseI and subjected to immunoprecipitation (IP) using α-GFP antibodies to pull down YFP-IN and associated complexes. RNA was separated from the immune complexes and subjected to RT-PCR to detect bound TAR RNA (FIG. 8e, panel a). Immunoblot analysis of bound proteins were carried out using: α-HA to detect bound HA-INI1 (FIG. 8e, panel b) and α-GFP to detect YFP-IN (FIG. 8e, panel c). RT-PCR of the RNA isolated from cell lysate was used as input control (FIG. 8e, panel d). Immunoblot analysis of total proteins from cell lysates as input control were carried out using α-HA to detect input HA-INI1 (FIG. 8e, panel e), α-GFP to detect input YFP-IN (FIG. 8e, panel f), and α-GAPDH to detect GAPDH as loading control (FIG. 8e, panel g). Results indicated that use of control IgG or lack of expression of GFP-IN or TAR RNA in 293T cells led to no RNA-co-IP (FIG. 8e lanes 1-4). Expression of both GFP-IN and TAR RNA in the cells led to successful RNA-co-IP by α-GFP antibodies (FIG. 8e, lane 5). Interestingly, addition of SP-38 but not SP-83(D225G) mutant peptide resulted in greater than 1000-fold inhibition of RNA binding in RNA-co-IP, while input levels of RNA were the same in the presence or absence of stapled peptides (FIG. e8, lanes 6 and 7). These studies established that SP-38 but not the mutant peptide SP-83, potently inhibits IN/RNA and IN/INI1 complex formation within cells.

### The SP38 Stapled Peptide Inhibits HIV-1 Replication at Low Micromolar Concentrations:

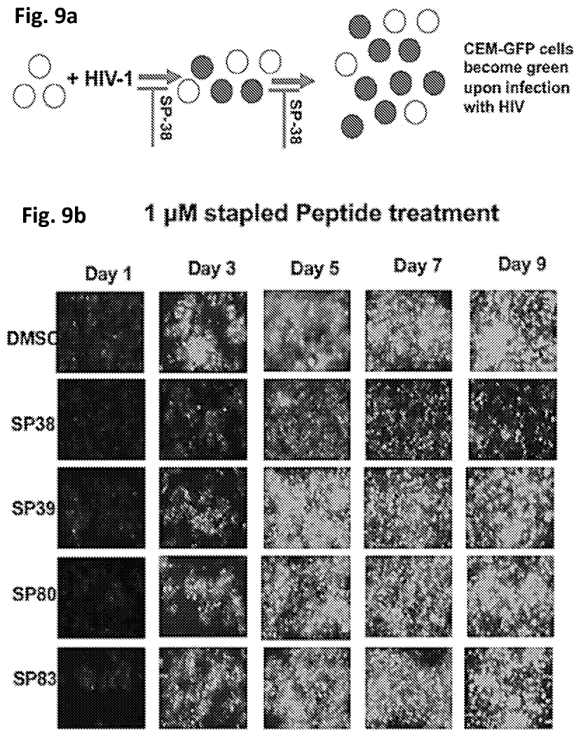

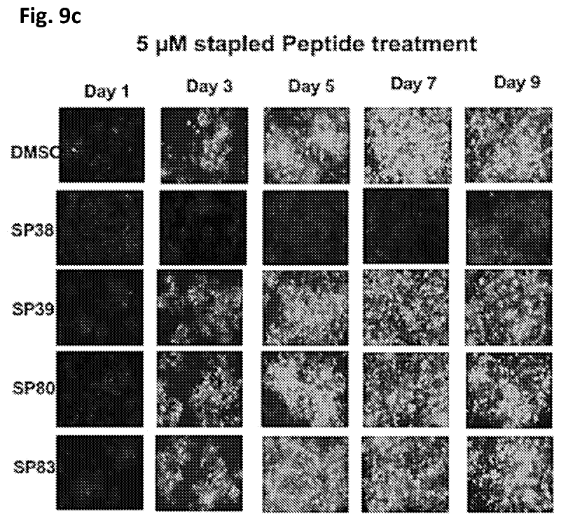

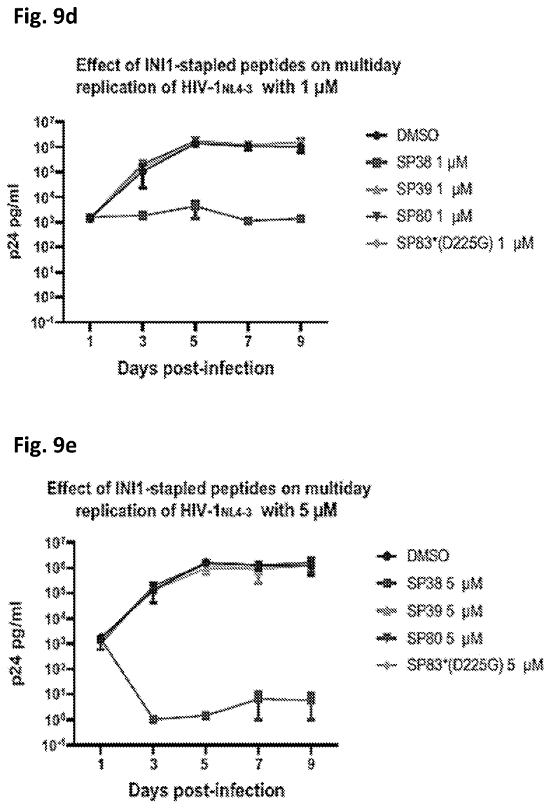

To determine if the stapled peptides can inhibit HIV-1 replication in cell culture, we used CEM-GFP cell lines, which harbor LTR-GFP as a reporter. HIV-1 infection of these cells activates GFP expression. By monitoring the GFP expression and by analyzing the p24 in the culture supernatants for virus production, we found that SP38 but not mutant peptide SP83 potently inhibited HIV-1 propagation in a multiday assay (FIG. 9). These results correlated with the ability of the peptides to inhibit IN/RNA and IN/INI1 interactions inside the transfected cells as shown in FIG. 8e.

### Mechanism of Inhibition Mediated by Stapled Peptides

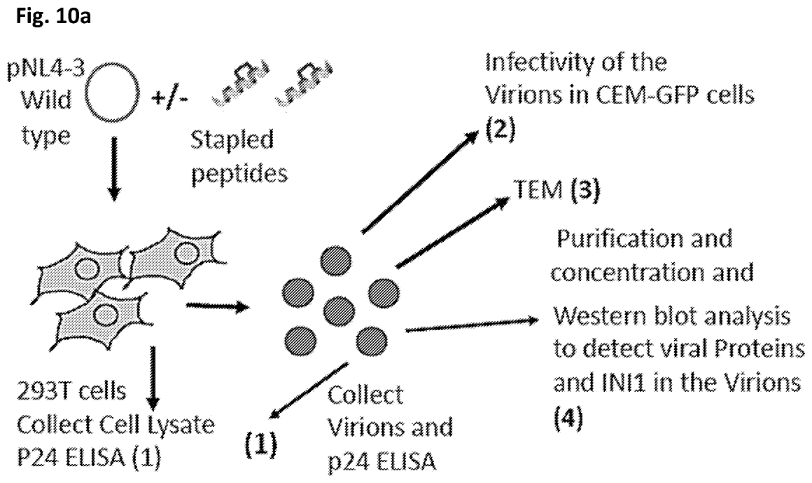

We hypothesized that INI1-derived Stapled peptides and drugs target IN and disrupt IN/RNA and IN/INI1 interactions. As a consequence of this inhibition, we expected that these stapled peptides and drugs would selectively inhibit virion particle morphogenesis during late events and that the morphologically defective particles would not be infectious, thus inhibiting HIV-1 replication. To test this hypothesis and to determine the mechanism of inhibition by stapled peptides, we tested the effect of stapled peptides on late events (production of HIV-1 particles and particle morphogenesis) and further tested the infectivity of particles that are produced (FIG. 10a). We found that stapled peptides do not inhibit particle production indicating that expression of HIV-1 viral RNA and viral proteins are not impaired by stapled peptides (FIG. 10b).

### Stapled Peptides Inhibit the Infectivity of Virions

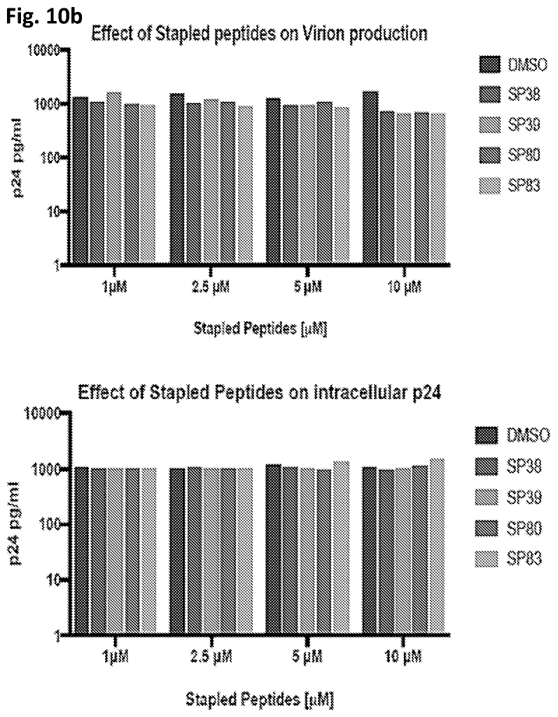

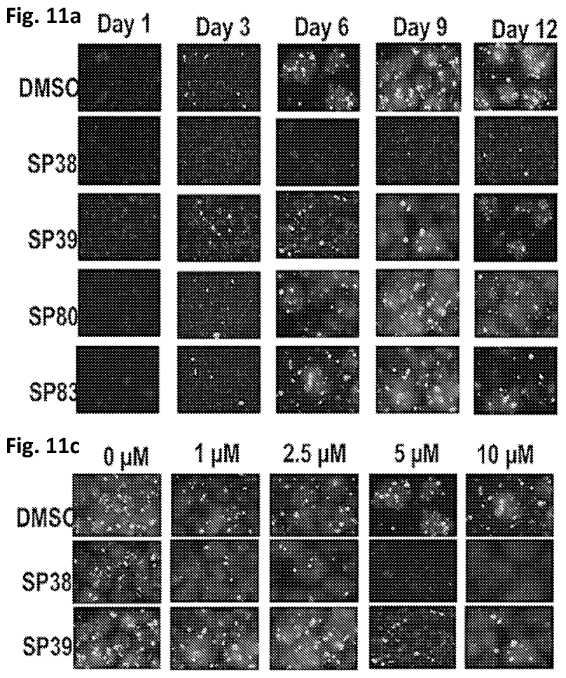

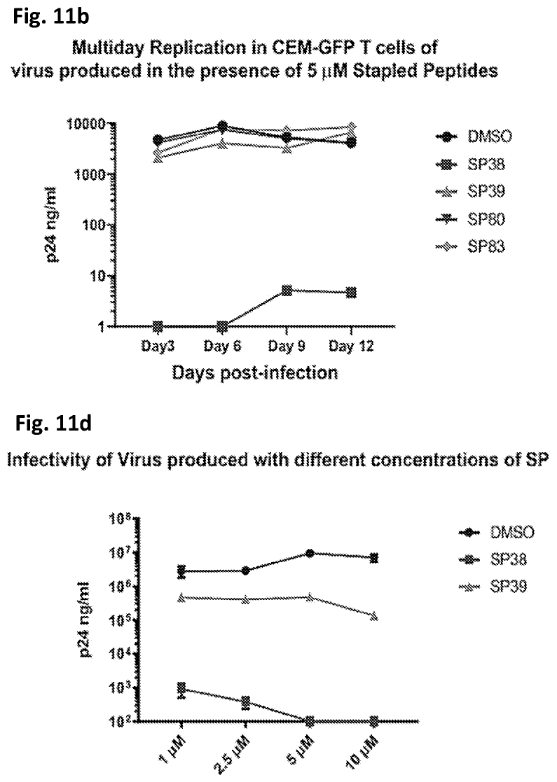

To determine the infectivity of virions produced in the presence of DMSO (DMSO-V), SP-38 (SP38-V), SP-80 (SP80-V) and SP83-D225G (SP83-V), we used equal amounts (5 ng p24) of virions to infect CEM-GFP cells in the absence of stapled peptides (FIGS. 11a and c). Furthermore, 5 ng p24 each of two sets of viruses produced in the presence of increasing concentrations (0, 1, 2.5, 5 and 10 microM) of peptides SP38 and SP39 were also used to infect CEM-GFP cells (FIGS. 11b and d). Replication of the virus was monitored by GFP expression in CMV-GFP cells and also by assaying for the production of p24 in the culture supernatants (FIGS. 11a-d). The results indicated that only SP38-V virions, but not control virions were defective for infection (FIGS. 11a-d).

### Stapled Peptides Inhibit the Particle Morphology of the Virions

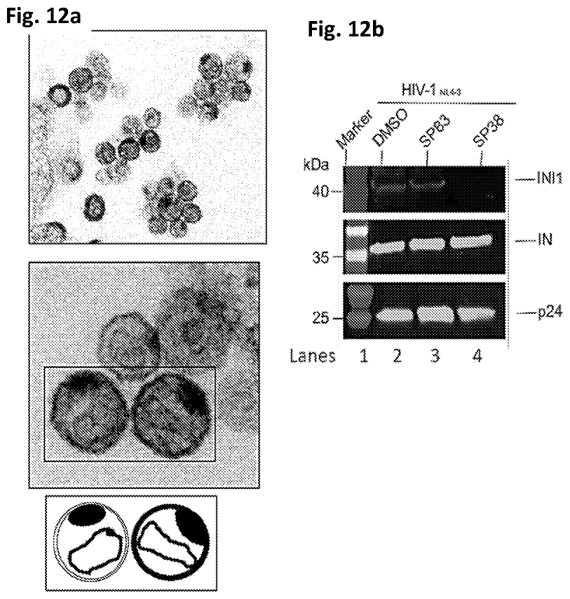

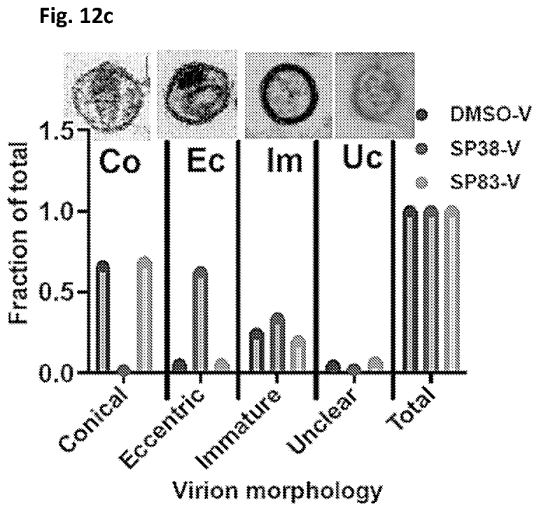

We have previously demonstrated that disruption of IN/INI1 interaction and IN/RNA interaction leads to defect in maturation of virion particles that results in production of “eccentric particles” that have electron dense material outside the capsid (Dixit et al. 2021). To determine if the virions produced in the presence of stapled peptides have similar defect in virion morphogenesis, we carried out TEM analysis of the virions (˜200 each). The results indicated that SP-38 treatment led to the production of a large number of morphologically defective “eccentric” particles and very few conical capsids, as opposed to controls where conical capsids were a majority (FIGS. 12a and c). These results established that the SP-38 did not affect viral protein expression, assembly, or particle production, but it selectively inhibited particle morphogenesis.

Stapled Peptides Inhibit Incorporation of INI1 into Virions

We tested the incorporation of INI1 into the virions. Electron microscopy analysis of ˜200 particles and Western analysis of concentrated virions were carried out to determine the presence or absence of INI1 (FIG. 12b). We found that INI1 was not incorporated into HIV-1 virions produced in the presence of SP38 but were incorporated into virions produced in the absence of peptides or in the presence of the control mutant peptide SP83 (FIG. 12b).

The above studies established that INI1-derived Stapled peptides disrupt IN-INI1 and IN-RNA interactions, and that they inhibit the particle morphogenesis and potently inhibit the infectivity of the particles.

### Stapled Peptides Inhibit the Infectivity of Viruses Produced in the Reactivated Latent Cells

The stapled peptides represents a novel class of drugs that selectively inhibit particle maturation to induce eccentric defective particles. This is an ideal drug to use in “shock and kill” therapy in combination with LRAs, where the LRAs reactivate latent virus, and produce virion particles. Since the stapled peptides did not inhibit viral RNA or protein expression, but inhibited the spread of the virus by generating morphologically defective virions, such drugs, when combined with LRAs, will be effective in preventing the spread of the reactivated virus.

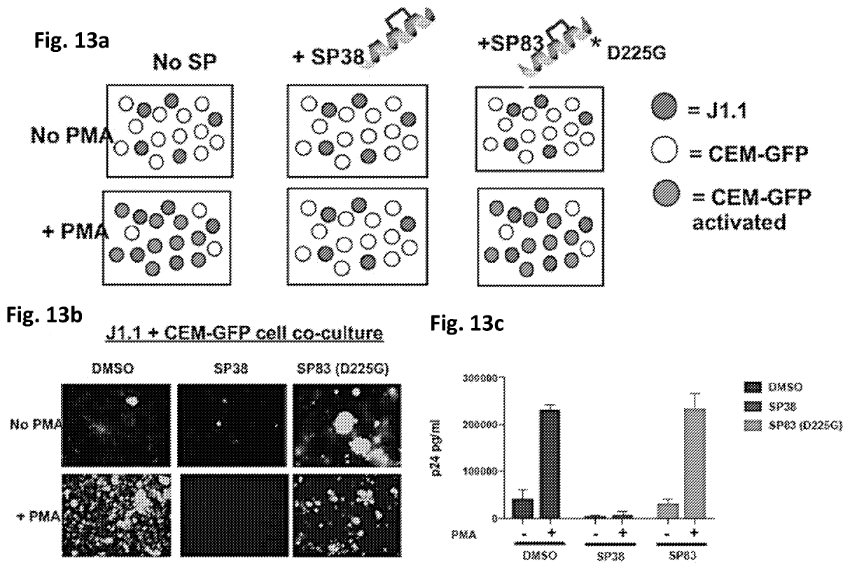

As a proof-of-concept to test if the stapled peptides inhibit spreading of the reactivated latent viruses, we used an in vitro cell line model of latency, J1.1 cells. J1.1 have a single latently integrated provirus which is activated using LRAs. We activated the latently integrated provirus by the addition of PMA and then mixed them with CEM-GFP cells in the cultures. Infection of CEM-GFP cells with virus produced from J1.1 upon reactivation turns the CEM-GFP T-cells green (FIGS. 13a and b right side panels). We tested the effect of addition of stapled peptides in this co-culture experiment (FIGS. 13a and b middle and left side panels). We found that SP38 but not SP83 mutant, inhibited the infection of CEM-GFP cells by the reactivated virus from latent cells as seen by the absence of green cells (FIGS. 13a and b middle and left side panels). We furthermore determined the p24 released from the culture supernatant as a measure of the virus production in the presence and absence of stapled peptides (FIG. 13c). Consistent with our hypothesis, SP38 but not Sp83 inhibited the virus spread in the culture (FIG. 13c).

Discovered herein is a novel concept that host factor INI1 mimics TAR RNA and both INI1 and TAR RNA bind the same surface of HIV-1 IN. As presented above, the binding of IN to RNA and INI1 are important for HIV-1 particle production and particle morphogenesis. Disrupting IN/RNA and IN/INI1 is a novel strategy to inhibit HIV-1 particle morphogenesis. Accordingly, we have developed new class of drugs based on the RNA-protein mimicry between INI1 and HIV-1 TAR RNA. We have developed stapled peptides derived from INI1 alpha1 helix that are dual acting to inhibit IN/INI1 and IN/RNA interactions. The stapled peptides impair the generation of infectious HIV-1 particles, without affecting particle production. The stapled peptides are “first-in-class” inhibitors of HIV-1 that disrupt the intracellular host-virus interactions between INI1 and IN and allows the production of reactivated virus from acutely or latently infected HIV-1 but inhibits the infectivity of these viruses, preventing them from spreading infection. This inhibition is due to the disruption of particle maturation that results in the production of morphologically defective HIV-1 virion particles. We predict that if the HIV-1 viruses were to develop resistant mutations as the viral escape mutants, it will be defective for replication. For example, even if the virus develops a mutation such that it no longer binds to the stapled peptide, and hence becomes resistant to the stapled peptide, the stapled peptide will also be defective for binding to both RNA and INI1. Thus, the resistant escape mutants will be defective for replication. This is the reason we propose that it will be hard to develop resistant viruses to stapled peptides.

### REFERENCES FOR EXAMPLES 2-6

1 Arts, E. J. & Hazuda, D. J. HIV-1 Antiretroviral Drug Therapy. Cold Spring Harb Perspect Med 2, a007161, doi:10.1101/cshperspect.a007161 (2012).
2 Lopez Angel, C. J. & Tomaras, G. D. Bringing the path toward an HIV-1 vaccine into focus. PLoS Pathog 16, e1008663, doi:10.1371/journal.ppat.1008663 (2020).
3 Vandegraaff, N. & Engelman, A. Molecular mechanisms of HIV integration and therapeutic intervention. Expert Rev Mol Med 9, 1-19, doi:S1462399407000257 [pii]10.1017/S1462399407000257 (2007).
4 Waheed, A. A. & Freed, E. O. HIV type 1 Gag as a target for antiviral therapy. AIDS Res Hum Retroviruses 28, 54-75, doi:10.1089/AID.2011.0230 (2012).
5 Siliciano, R. F. & Greene, W. C. HIV latency. Cold Spring Harb Perspect Med 1, a007096, doi:10.1101/cshperspect.a007096 (2011).
6 Khanal, S., Schank, M., El Gazzar, M., Moorman, J. P. & Yao, Z. Q. HIV-1 Latency and Viral Reservoirs: Existing Reversal Approaches and Potential Technologies, Targets, and Pathways Involved in HIV Latency Studies. Cells 10, doi:10.3390/cells10020475 (2021).
7 Ait-Ammar, A. et al. Current Status of Latency Reversing Agents Facing the Heterogeneity of HIV-1 Cellular and Tissue Reservoirs. Front Microbiol 10, 3060, doi:10.3389/fmicb.2019.03060 (2019).
8 Cano, J. & Kalpana, G. V. Inhibition of Early Stages of HIV-1 Assembly by INI1/hSNF5 Transdominant Negative Mutant S6. J Virol 85, 2254-2265, doi:JVI.00006-10 [pii] 10.1128/JVI.00006-10 (2011).
9 Kalpana, G. V., Marmon, S., Wang, W., Crabtree, G. R. & Goff, S. P. Binding and stimulation of HIV-1 integrase by a human homolog of yeast transcription factor SNF5 [see comments]. Science 266, 2002-2006 (1994).
10 La Porte, A., Cano, J., Wu, X., Mitra, D. & Kalpana, G. V. An Essential Role of INI1/hSNF5 Chromatin Remodeling Protein in HIV-1 Posttranscriptional Events and Gag/Gag-Pol Stability. J Virol 90, 9889-9904, doi: 10.1128/JVI.00323-16 (2016).
11 Morozov, A. et al. INI1 induces interferon signaling and spindle checkpoint in rhabdoid tumors.
   Clin Cancer Res 13, 4721-4730, doi:13/16/4721 [pii]
   10.1158/1078-0432.CCR-07-0054 (2007).
12 Morozov, A., Yung, E. & Kalpana, G. V. Structure-function analysis of integrase interactor 1/hSNF5L1 reveals differential properties of two repeat motifs present in the highly conserved region. Proc Natl Acad Sci USA 95, 1120-1125 (1998).
13 Sorin, M., Yung, E., Wu, X. & Kalpana, G. V. HIV-1 replication in cell lines harboring INI1/hSNF5
   mutations. Retrovirology 3, 56, doi:1742-4690-3-56 [pii]10.1186/1742-4690-3-56 (2006).
14 Yung, E. et al. Inhibition of HIV-1 virion production by a transdominant mutant of Integrase interactor 1. Nature Med. 7, 920-926 (2001).
15 Yung, E. et al. Specificity of interaction of INI1/hSNF5 with retroviral integrases and its functional significance. J Virol 78, 2222-2231 (2004).
16 Kim, D. E., Chivian, D. & Baker, D. Protein structure prediction and analysis using the Robetta server. Nucleic Acids Res 32, W526-531, doi:10.1093/nar/gkh468 (2004).
17 Laskowski, R. A., Rullmannn, J. A., MacArthur, M. W., Kaptein, R. & Thornton, J. M. AQUA and PROCHECK-NMR: programs for checking the quality of protein structures solved by NMR. J. Biomol. NMR 8, 477-486 (1996).
18 Vriend, G. WHAT IF: a molecular modeling and drug design program. J Mol Graph 8, 52-56, 29, doi:10.1016/0263-7855(90)80070-v (1990).
19 Eisenberg, D., Luthy, R. & Bowie, J. U. VERIFY3D: assessment of protein models with three-dimensional profiles. Methods Enzymol 277, 396-404, doi:10.1016/s0076-6879(97)77022-8 (1997).
20 Wiederstein, M. & Sippl, M. J. ProSA-web: interactive web service for the recognition of errors in three-dimensional structures of proteins. Nucleic Acids Res 35, W407-410, doi:10.1093/nar/gkm290 (2007).
21 Eijkelenboom, A. P. et al. Refined solution structure of the C-terminal DNA-binding domain of human immunovirus-1 integrase. Proteins 36, 556-564 (1999).
22 Eijkelenboom, A. P. A. M. et al. The DNA-binding domain of HIV-1 integrase has an SH3-like fold. Nature Structural Biology 2, 807-810 (1995).
23 Xu, X. et al. Performance of MDockPP in CAPRI rounds 28-29 and 31-35 including the prediction of water-mediated interactions. Proteins 85, 424-434, doi:10.1002/prot.25203 (2017).
24 Huang, S. Y. et al. Inclusion of the orientational entropic effect and low-resolution experimental information for protein-protein docking in Critical Assessment of PRedicted Interactions (CAPRI). Proteins 81, 2183-2191, doi:10.1002/prot.24435 (2013).
25 Huang, S. Y. & Zou, X. A knowledge-based scoring function for protein-RNA interactions derived from a statistical mechanics-based iterative method. Nucleic Acids Res 42, e55, doi:10.1093/nar/gku077 (2014).

### Example 7: INI1/SMARCB1 Rpt1 Domain Mimics TAR RNA in Binding to Integrase to Facilitate HIV-1 Replication

As disclosed herein, INI1/SMARCB1 binds to HIV-1 integrase (IN) through its Rpt1 domain and exhibits multifaceted role in HIV-1 replication. Determining the NMR structure of INI1-Rpt1 and modeling its interaction with the IN-C-terminal domain (IN-CTD) reveal that INI1-Rpt1/IN-CTD interface residues overlap with those required for IN/RNA interaction. Mutational analyses validate our model and indicate that the same IN residues are involved in both INI1 and RNA binding. INI1-Rpt1 and TAR RNA compete with each other for IN binding with similar IC50 values. INI1-interaction-defective IN mutant viruses are impaired for incorporation of INI1 into virions and for particle morphogenesis. Computational modeling of IN-CTD/TAR complex indicates that the TAR interface phosphates overlap with negatively charged surface residues of INI1-Rpt1 in three-dimensional space, suggesting that INI1-Rpt1 domain structurally mimics TAR. This possible mimicry between INI1-Rpt1 and TAR explains the mechanism by which INI1/SMARCB1 influences HIV-1 late events and suggests additional strategies to inhibit HIV-1 replication.

INI1/SMARCB1/hSNF5/BAF47 is an invariant component of the SWI/SNF chromatin remodeling complex, involved in a multitude of cellular functions, including transcription, cell cycle regulation, development, and tumor suppression^{1,2}. INI1 and SWI/SNF are frequently mutated in cancers^{1,2}. INI1 was first identified as a binding partner for HIV-1 integrase (IN)³, and studies suggest that it is required at multiple stages of HIV-1 replication including integration, HIV-1 transcription, post transcriptional Gag RNA and protein stability, virus assembly, and particle production⁴⁻¹⁴. Interestingly, HIV-1 IN also influences multiple stages of viral replication, including integration and particle morphogenesis¹⁵⁻¹⁸.

INI1/SMARCB1 interacts with various viral and cellular proteins^{8,19-23} via its two highly conserved imperfect repeat domains, Rpt1(aa 183-248) and Rpt2(aa 259-319), connected by a linker region (aa 249-258) (FIG. 1a)²⁴. Rpt1 (but not Rpt2) is necessary and sufficient for binding HIV-1 IN²⁴. INI1 is selectively incorporated into HIV-1, but not other lentiviral or ret-roviral particles¹¹. Furthermore, an INI1 fragment termed S6 (aa 183-294) harboring the Rpt1 domain, linker region and a part of Rpt2, trans-dominantly inhibits HIV-1 particle production by binding to IN within GagPol¹⁰. These studies indicate that INI1 is required for HIV-1 late events. INI1 influences integration in vitro depending on IN-INI1 stoichiometry, and the addition of SWI/SNF complex enhances integration into nucleosomal targets^{3,5,13}. How INI1 binds to IN and influences multiple stages of HIV-1 replication is not clearly understood.

As disclosed herein, we provide the solution structure of the conserved Rpt1+linker domain _{(INI1183-265)} of INI1 (PDB ID: 6AX5) and molecular docking of the IN-CTD/INI1-Rpt1 complex. These studies indicate that the IN residues at the IN/INI1 interface are the same as those needed for the interaction of IN with the TAR region of the HIV-1 genome¹⁸. IN/RNA interaction is necessary for particle morphogenesis^{15,18}. Interestingly, IN mutants defective for binding to INI1 also affect particle morphogenesis⁷. Our current analysis indicates that there is a remarkable similarity between INI1-Rpt1 and TAR RNA with regard to their binding to IN-CTD. Comparison of the negative charges on the electrostatic surfaces of the INI1-Rpt1 and the TAR RNA NMR structures, and modeling of the IN-CTD/TAR RNA complex by molecular docking and its comparison to IN-CTD/INI1-Rpt1 complex reveals that INI1-Rpt1 may structurally mimic TAR RNA. The structural mimicry between INI1-Rpt1 and TAR RNA explains the multifaceted role of INI1/SMARCB1 during HIV-1 replication in vivo and provides mechanistic insights into INI1-IN interactions.

### Example 8: NMR Structure of INI1₁₈₃₋₂₆₅ and Modeling INI1₁₈₃₋₃₀₄

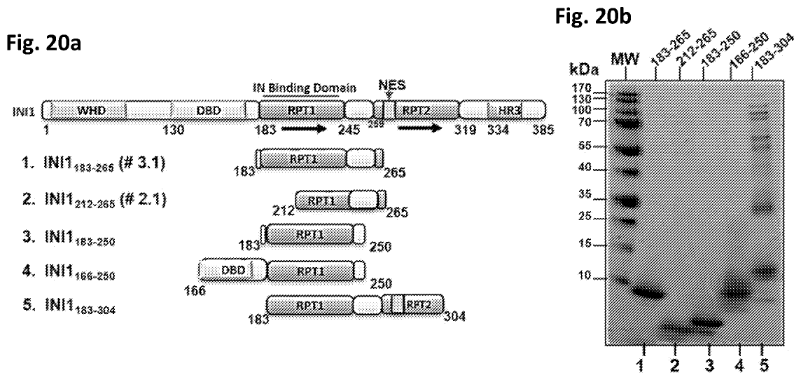

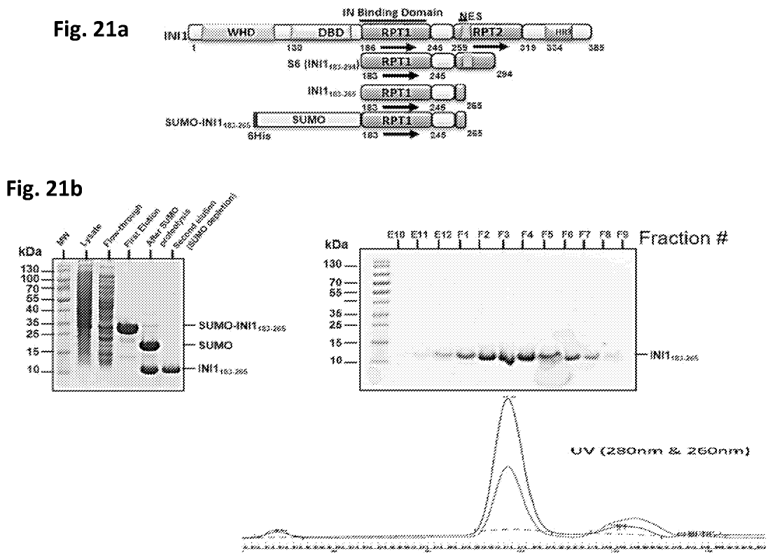

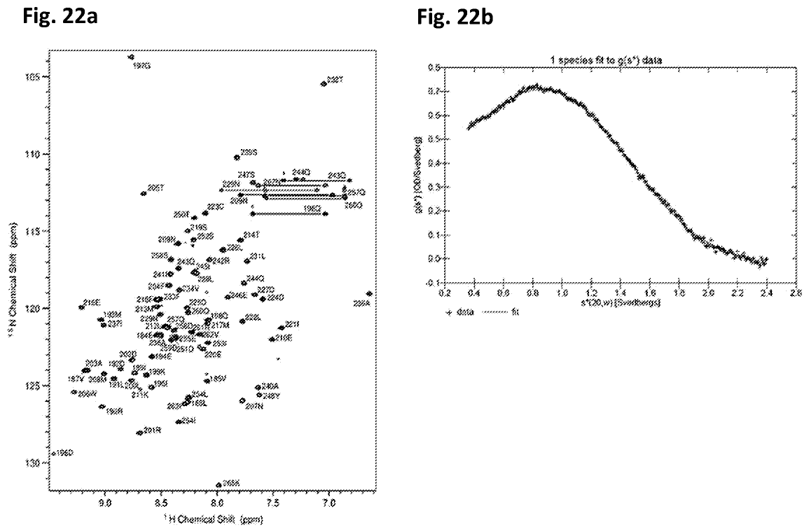

We selected INI1₁₈₃₋₂₆₅ for NMR study after screening several overlapping fragments harboring the Rpt1 domain, as this fragment exhibited good solution property (FIG. 1a and FIGS. 20 and 21). The uniformly ¹³C,¹⁵N-labeled INI1183-265 fragment was subjected to NMR analysis. The assigned ¹H1⁵N HSQC spectrum for non-deuterated _{INI1185-265} is shown in FIG. 22b. Unlike the multimeric full-length protein, the INI1183-265 domain is monomeric in solution as judged by both NMR self-diffusion and analytical ultracentrifugation measurements (FIG. 22b). The _{INI1183-265} fragment consists of the Rpt1 domain (aa183-248) and the linker region (aa 249-265) between Rpt1 and Rpt2. The NMR structure indicated the presence of a well-ordered Rpt1 domain-containing (3(3αα topology and a disordered linker segment [FIG. 1b, c, Table 1, and PDB ID: 6AX5]. Superimposition of our structure (6AX5) to other existing NMR, X-ray crystal, and cryoEM structures [5L7A, 5L7B, 5GJK, and 6LTJ] indicated that the (3(3αα core region of 6AX5 perfectly aligns with the other structures with an RMSD (root-mean-square deviation) of −1.0 Å (FIG. 1d and Table 2).

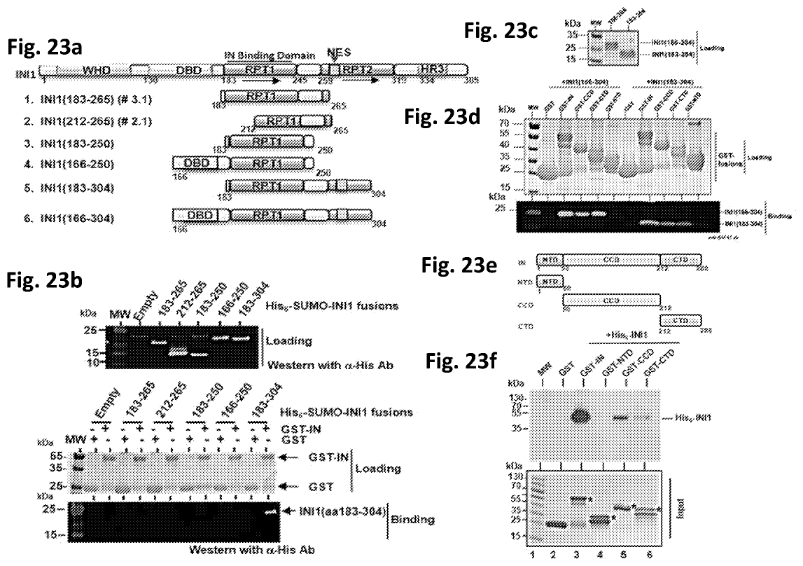

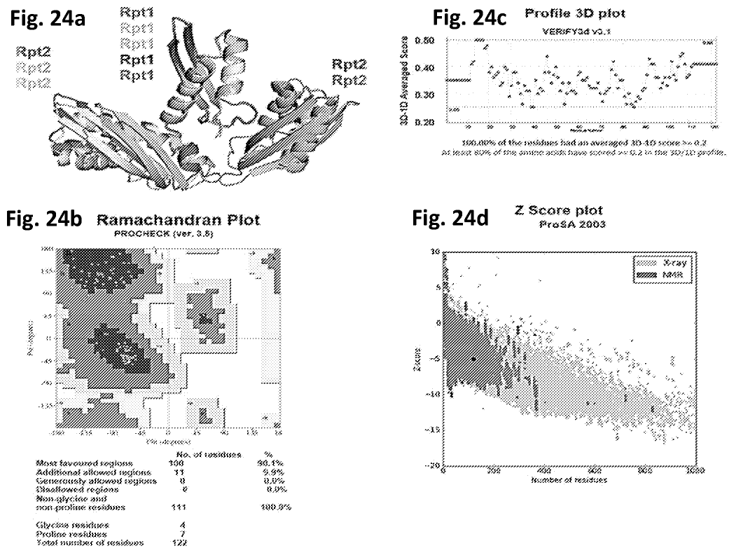

While Rpt1(INI1₁₈₃₋₂₄₅) is sufficient for IN binding, a longer fragment S6(INI1₁₈₃₋₂₉₄) harboring Rpt1+linker+partRpt2, shows stronger binding²⁴ and acts as a dominant-negative inhibitor of HIV-1¹⁰. Consistent with this, the fragments INI1183-304 and INI1₁₆₆₋₃₀₄ but not INI1₁₈₃₋₂₆₅ interacted strongly with full-length IN and central core (IN-CCD, aa 50-200) and C-terminal (IN-CTD, aa 201-288) domains in vitro (FIG. 23a-f). To determine the contribution of INI1-Rpt2 for IN binding, we modelled _{INI1183-319} fragment containing Rpt1-linker-Rpt2 based on 6AX5 structure using Robetta²⁵. This modeling simulation yielded five best clusters, each possessing well-ordered Rpt1 and Rpt2 domains. The Rpt2 region was topologically identical to Rpt1 and the five clusters differed from each other only in the linker region (FIG. 24a and Supplementary Data 1). We also modeled the structure of the strong binding _{INI1183-304} fragment by using Robetta²⁵, which showed that Rpt1 and Rpt2 domains were symmetrically arranged separated by the linker region (FIG. 1e and Supplementary Data 1). Further ab initio modeling of linker region in INI1183-304 exhibited significantly greater order than that observed in the NMR structure that lacked the Rpt2 (FIG. 1e). This modeled structure was validated using various tools including Ramachandran plot, PROCHECK version 3.5²⁶ WHATIF ver. 8.0²⁷, VERIFY3D v3.1²⁸, and PROSA 2003²⁹ (FIG. 24b-d), which strongly supported the features of the model. The highest scoring modeled _{INI1183-304} structure was used to study its interaction with IN. Analysis of the Rpt1 portion of this model with the existing structures exhibited RMSD of −1.0 Å (Table 3), and Rpt2 portion of this model exhibited RMSD of 1.25 Å with the cryoEM structure³⁰ (PDBID:6LTJ).

### Example 9: Interaction of INI1 and IN is Mediated by Extensive Hydrophobic and Complementary Ionic Interactions

While INI1183-304 fragment strongly binds to IN-CCD and IN-CTD domains (FIG. 23), the INI1₁₈₃₋₃₀₄/IN-CTD complexes were large and hence were not amenable for NMR studies. Therefore, we computationally docked the INI1183-304 structure with the NMR structure of IN-CTD [(1QMC)]^{31,32} using HADDOCK, without interaction restraints (FIG. 14 and Supplementary Data 1). The docked complex with the lowest (best) energy HADDOCK score of −184.3±21.5 indicated that the Rpt1 portion within the INI1183-304 fragment has the potential to directly interact with IN-CTD (FIG. 14a). The exposed negatively charged Rpt1 residues of INI¹183-304 contacted the basic residues of IN-CTD, and the interaction of hydrophobic residues of the two proteins resulted in a hydrophobic core within the complex (FIG. 14b,c and f). Upon complex formation, −642 Å² of solvent-accessible surface was buried, which is similar in size to buried surface area in the comparable SWIRM/INI1 Rpt1 complex [699.1 Å²]³³.

This docking study suggested that the INI1-Rpt1/IN-CTD interaction was mediated by both hydrophobic and specific ionic interactions (FIG. 14d-f). In the model of the complex, acidic residues D225 and D224 from α1 of INI1-Rpt1 made polar contacts with basic IN-CTD residues R228 (in strand (31), K264, and R263 (in the loop between (34 and (35 strands) (FIG. 14d). Additional ionic interactions were noted between INI1183-304 E210 (in the loop region between (32 and α1) and IN-CTD residues R262 (in the loop between (34 and (35 strands) and K244 (in the loop between (32 and (33 strands) (FIG. 14f). The IN-CTD W235 residue formed the center of the Rpt1/CTD interface, surrounded by a shallow hydrophobic patch/cage formed by F204, L226, L222, I221, and F228 residues from α1 of Rpt1 (FIG. 14e, f). This channel is surrounded by the ionic interactions formed between INI1-Rpt1 residues D225, D224 with IN-CTD residues R228, K264, and R263 at one end; between E210 of INI1 with K244 and R262 of IN-CTD, and between E184 of INI1 and R269 of IN-CTD residues at the other end. The validity of this model was confirmed by biochemical studies of interface residue mutants as follows.

### Example 10: Biochemical Interaction Studies Validate the INI1₁₈₃₋₃₀₄/CTD Model

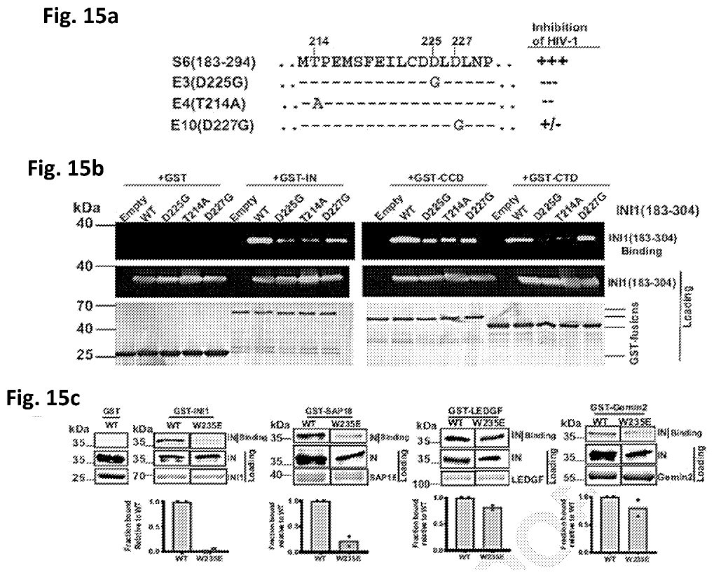

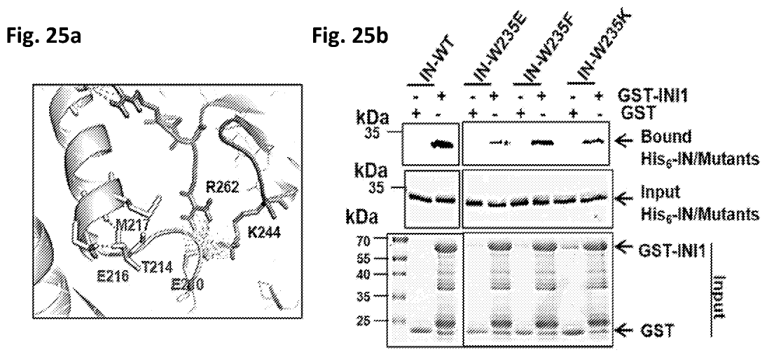

Previous reverse yeast-two hybrid genetic screening using a random mutation library identified D225G, T214A, and D227G (termed E3, E4, and E10) mutations in _{s6(IN11183-294)} that disrupted its ability to interact with integrase and inhibit HIV-1 particle production (FIG. 15a)¹⁰. D225G and T214A mutants were most defective for binding and inhibition, while the D227G mutant was less defective¹⁰. In our model, while D225 residue is at one end of a1 helix and makes ionic interactions with IN R228 and K264, T214 is at the other end of the INI1-Rpt1 α1 helix, facing the binding interface (FIG. 15d and FIG. 25a). Therefore, mutating these residues would disrupt the IN/INI1 interaction or destabilize the α1 helix. Interestingly, INI1-Rpt1 D227 faces away from the binding interface and substituting this residue should cause minimal disruption of the interaction (FIG. 14d). To test these predictions, we determined the interactions of GST-fusions of full-length IN, CCD, and IN-CTD with INI1183-304 wild type and D225G, D227G, T214A mutants (FIG. 15b). We found that INI1183-304 mutants D225G and T214A were highly defective and D227G mutant was least defective, for binding to IN and IN-CTD, consistent with the prediction of the model (FIG. 15b).

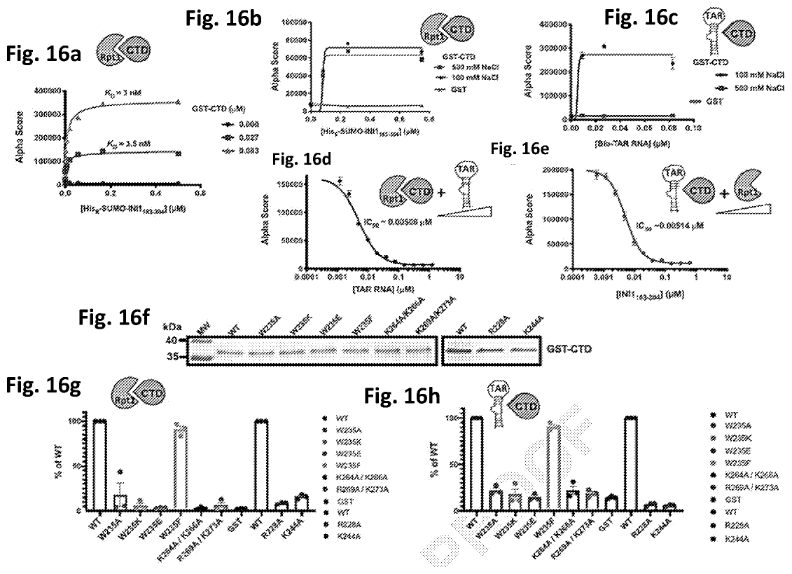

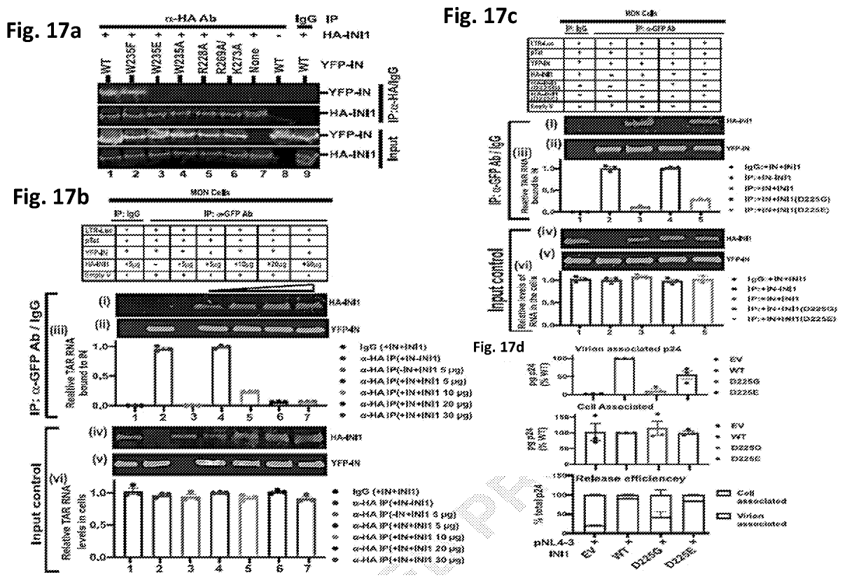

Our model also predicts that W235 residue is nestled in a hydrophobic cage, and substituting this residue with a charged but not with another aromatic residue would disrupt IN/INI1 interactions (FIG. 14d, e). Interestingly, previous reports have indicated that W235E and W235K, but not W235F substitution mutations selectively inhibit integration activity in vivo but not in vitro³⁴⁻³⁶. The reason for the differential effects of these mutants in vivo are not well understood. We carried out an in vitro GST-pull down assay to determine if the phenotypes observed for W235 mutants correlate with their ability to interact with INI1. IN-W235E mutant was defective for interaction with GST-INI1 (FIG. 15c). To determine the specificity, we also tested the interaction of IN-W235E mutant with GST-fusions of other binding partners of IN, namely LEDGF, Gemin2 and SAP18. Our data indicated that while wild type His6-IN binds to all four proteins, the His6-IN-W235E mutant was highly defective for binding to GST-INI1 (<5% binding), partially defective for binding to GST-SAP18 (˜20% binding) and was not significantly defective for binding to GST-fusions of LEDGF and GEMIN2 (FIG. 15c). These studies suggested that W235 residue is specifically important for interaction of IN with INI1. We next tested the ability of W235E, W235K, and W235F substitutions to interact with GST-IN. We found that while the W235F mutant retained the interaction with INI1 as strongly as the wild type, W235K and W235E mutants showed reduced interactions in vitro (FIG. 25b). These results were also validated by Alpha proximity assay^{37,38} and co-immunoprecipitations as described below (FIGS. 16 and 17).

### Example 11: Similarity Between the INI1₁₈₃₋₃₀₄ and TAR RNA for Binding to IN

During these studies, we unexpectedly noted that some of the critical residues at the interface of the IN-CTD/_{INI1183-304} complex, such as K264, R269, were the same residues shown to be important for the interaction of IN with HIV-1 genomic RNA15,18 (FIG. 14d, f). These mutations affect binding of the TAR region of HIV-1 genomic RNA to IN and lead to defective particle morphogenesis¹⁸. Our previous studies have indicated that INI1-interaction-defective-(IID) IN mutants in the core domain also lead to defects in particle morphogenesis⁷. Based on these observations, we hypothesized that INI1 and TAR RNA could bind to the same residues of IN-CTD and may have overlapping functions in particle morphogenesis. To test this hypothesis, we determined if. (i) INI1₁₈₃₋₃₀₄ and TAR compete with each other for binding to IN in vitro; (ii) IN-CTD mutations affect the binding of _{INI1183-304} and TAR to the same extent; (iii) INI1 can compete with IN binding to TAR in vivo; and (iv) INI1-interaction defective W235E and R228A mutant viruses form morphologically defective particles and are defective for incorporation of INI1 into the virnons.

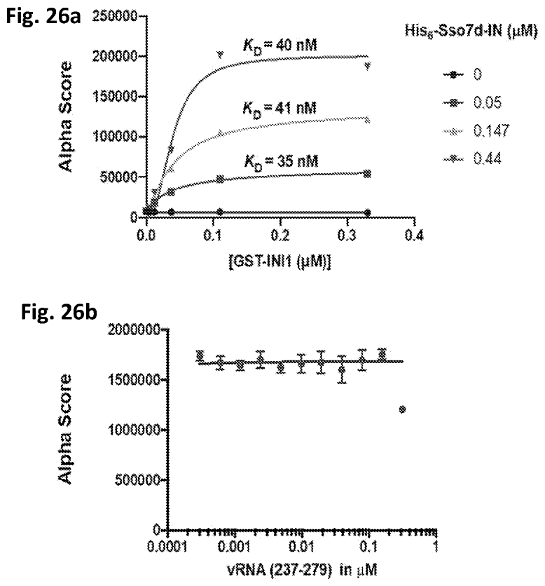

We established a quantitative protein-protein interaction Alpha proximity assay^{37,38} to assess the interaction of IN/INI1 and IN-CTD/INI1₁₈₃₋₃₀₄, the results of which showed low nM K_{D} for binding (FIG. 16a and FIG. 26a). While IN-CTD/INI1₁₈₃₋₃₀₄ interactions were unaffected by salt, consistent with the previous results¹⁸, IN-CTD/TAR interaction was sensitive to high NaCl (500 nM) (FIG. 16b, c).

First, we used Alpha assay to determine if there is competition between TAR RNA and _{INI1183-304} for binding to GST-CTD. Constant amounts of either Biotinylated-TAR or His6-SUMO-INI1₁₈₃₋₃₀₄ were incubated with GST-CTD, in the presence of increasing concentration of a third molecule (either INI1₁₈₃₋₃₀₄ or TAR RNA) under low salt conditions^{37,38}. We found that TAR RNA and _{INI1183-304} competed with each other for binding to IN-CTD with similar IC₅₀ value (≈5 nM, FIGS. 16d and e). The competition was specific, as another fragment of HIV-1 RNA (nts 237-279), containing a similar stem and loop content did not significantly inhibit the interaction between _{INI1183-304} and IN-CTD under these conditions (FIG. 26b). These results indicated that TAR and INI1183-304 compete with each other to bind to IN-CTD.

Second, we tested to determine if IN-CTD shows the same profile of interactions with both INI1₁₈₃₋₃₀₄ and TAR RNA. We tested the effect of a panel of IN-CTD substitution mutants from the IN-CTD/INI1₁₈₃₋₃₀₄ interface, R228A, K244A, W235E, W235K, W235F, W235A and the two mutants K264A/K266A and R269A/K273A that have been shown to block IN/TAR binding¹⁸ for their ability to interact with INI1₁₈₃₋₃₀₄ and TAR RNA at low salt conditions using Alpha assay. Our results indicated that all the mutant IN-CTD proteins tested, except for W235F, were defective for binding to INI1₁₈₃₋₃₀₄ consistent with our model (FIG. 16f, g). Interestingly, the same panel of mutants (except for W235F) were also defective for binding to TAR to the same extent (FIG. 16h). W235F IN-CTD mutant was not defective for binding to either TAR-RNA or INI1₁₈₃₋₃₀₄ whereas other mutants were defective for binding to both, suggesting that _{INI1183-304} and TAR RNA recognize the same residues for binding.

Third, to determine if the in vitro results can be recapitulated in vivo, we carried out co-immunoprecipitation (co-IP) and RNA-co-immunoprecipitation (RNA-IP) to test the interactions of full-length IN with INI1 and HIV-1 RNA in vivo. HA-INI1 and YFP—IN or YFP-IN mutants (W235E, W235F, W235A, R228A, R269A/K273A) were co-transfected into 293 T cells and immunoprecipitated using α-HA antibodies. All the mutants of IN and INI1 were expressed at comparable levels in cells as shown by the input control (FIG. 17a, lower two panels). α-HA antibodies immunoprecipitated equal amounts of INI1 in all the samples (FIG. 17a, second panel from the top). INI1 was able to co-immunoprecipitate WT IN and IN(W235F) (FIG. 17a, lanes 1 and 2, upper panel) but not the other IN mutants. Control samples where either INI1 or IN were missing or when isotype IgG antibody was used, showed no co-immunoprecipitation (FIG. 17a, lanes 7-9). These results establish that the IN residues identified at the interface are important for full-length IN-INI1 interaction in vivo.

We next determined if INI1 and TAR RNA competed with each other for binding to IN in vivo by RNA-co-IP in MON (INI1⁻/⁻) cells⁹. TAR RNA was expressed by transfecting pLTR-luc and pCMV-Tat in the presence or absence of YFP-IN and HA-INI1. Lysates of transfected MON cells were treated with DNase I to remove residual DNA and subjected to IP by using α-GFP antibodies to pull down YFP-IN and associated complexes. Protein and RNA were separated from the immune complexes, RNA was subjected to RT-PCR to detect viral RNA, and proteins were subjected to Western blot analysis to detect the presence of YFP-IN and HA-INI1 using α-GFP and α-HA antibodies. The results indicated that while IgG did not co-immunoprecipitate either INI1 or TAR-RNA (FIG. 17b, lane 1), α-GFP antibodies were able to pull down YFP-IN, which in turn, was able to RNA-co-IP TAR RNA in the absence of INI1 (FIG. 17b, lane 2). The presence of HA-INI1 led to co-immunoprecipitation of both INI1 and TAR RNA by IN (FIG. 17b, lane 4). To determine if INI1 and TAR RNA competed with each other for binding to IN, we transfected increasing concentrations of INI1 and a constant amount of IN. We found that increasing INI1 decreased the amount of bound TAR RNA but increased the amount of bound INI1 (FIG. 17b lanes 5-7, top three panels). We also ran all the input controls that showed that there was a uniform loading of IN and RNA (FIG. 17b, bottom three panels).

We additionally found that IN-binding-defective INI1 mutant (D225G) was unable to compete with TAR RNA to bind to IN (FIG. 17c, lane 4). However, mutant (D225E) with a conservative substitution, behaved like the wildtype and inhibited binding of TAR to IN (FIG. 17c, lane 5), suggesting that a negatively charged residue at 225 position is important for INI1 binding to IN. These results support the hypothesis that INI1 and TAR bind to IN and compete with each other in vivo.

To validate the IN-INI1 interaction model and to establish that INI1 interaction with IN is necessary for viral replication, we carried out complementation assay in INI1⁻/⁻ MON cells. Previously we demonstrated that lack of INI1 leads to a defect in viral particle production and that co-expression of INI1 complements this defect^{6,9}. We tested the function of INI1 mutants D225G and D225E on their ability to support particle production in INI1−/− MON cells. The results indicated that there was a defect in particle production in the absence of INI1 (FIG. 17d, EV=Empty vector). Co-transfection of wild type INI1, but not the IN-interaction-defective INI1(D225G) mutant, lead to particle production (FIG. 17d). On the contrary, the INI1(D225E) mutant significantly increased the particle production compared to the empty vector (FIG. 17d). These results are consistent with the observation that D225G does not bind to IN, and support the hypothesis that INI1 binding to IN is required for particle production.

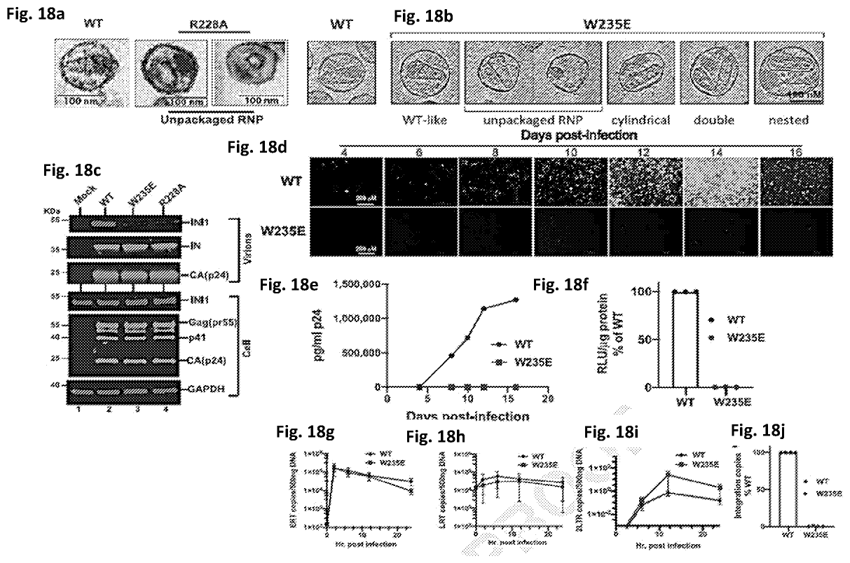

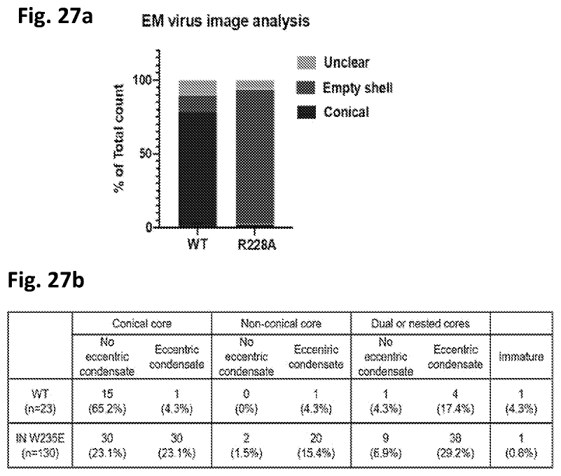

Finally, to determine the effect INI1-interaction defective mutations on particle morphogenesis, we produced W235E and R228A IN mutants of HIV-1_{NL4-3} in 293 T cells and carried out transmission Electron microscopy (TEM) and Cryo-Electron tomography (Cryo-ET) studies (FIGS. 18a and b). The EM structure of R228A virus demonstrated the presence of empty capsids with unpackaged ribonucleoprotein (RNP) appearing as eccentric electron dense material in ˜90% of virions (n=186), consistent with it being a class II IN mutant³⁹ (FIG. 18a and FIG. 27a). The Cryo-ET data indicated that W235E mutant particles (n=130) also resembled other class II IN mutants and a WT sampling (n=23) was consistent with previously characterized HIV-1 WT virion morphology^{40,41} (FIG. 18b and FIG. 27b). Compared to WT, W235E virions more frequently contained abnormal cores (54% versus 21%) or eccentric condensates of unpackaged RNP (68% versus 26%). W235E mutants were three-fold less likely to exhibit WT-like morphology, with RNP properly encapsidated in a conical core (23% versus 65%). Notably, conical capsids were over seven times more likely to contain RNP, with 81 filled out of 112 (72%), compared to cylindrical capsids, with only 5 filled of 52 (9.6%) (FIG. 18b and FIG. 27b). These findings together suggested that R228A and W235E mutations disrupted HIV IN role in particle morphogenesis.

It is known that INI1 is selectively incorporated into HIV-1 virions^{10,11}. We tested to determine if INI1/IN interaction is necessary for INI1 incorporation into virions using W235E and R228A mutants. Concentrated virions and the corresponding producer cell lysates were subjected to Western analysis to detect viral proteins and INI1 (FIG. 18c). We found that wild type virus (FIG. 18c lane 2), but not the W235E and R228A mutant virions incorporated INI1, indicating that binding to IN is necessary for this incorporation (FIG. 18c lanes 3 and 4).

Further virological analysis of W235E mutants in both multiday and single cycle infection assays, investigating various post-entry events [Early RT(ERT), Late RT (LRT), nuclear localization and integration] confirmed several reports of a selective defect in integration in vivo^{34,42} (FIG. 18d-j). Our virological studies provided additional information that the W235E mutant is defective for particle morphogenesis, while confirming previous results of a selective defect in integration.

### Example 12: Modeling TAR/IN-CTD Complex and Comparative Analysis Reveals the Structural Basis of Similarity of INI1₁₈₃₋₃₀₄ and TAR Binding to IN-CTD

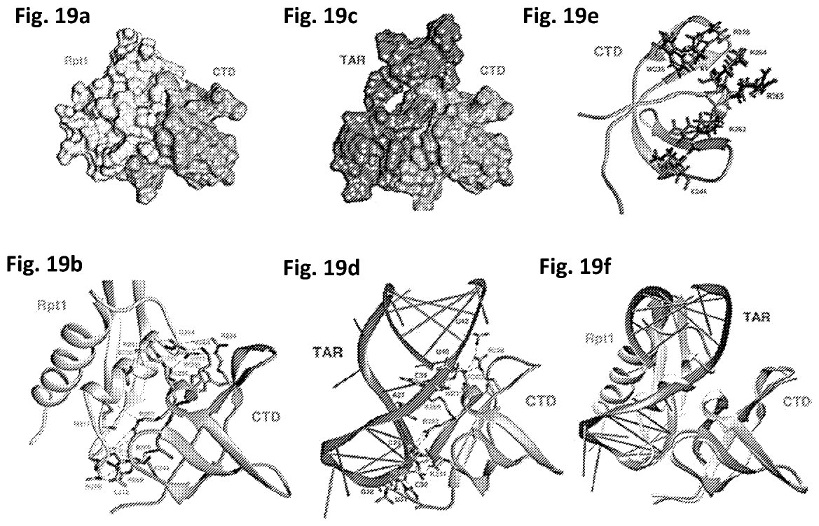

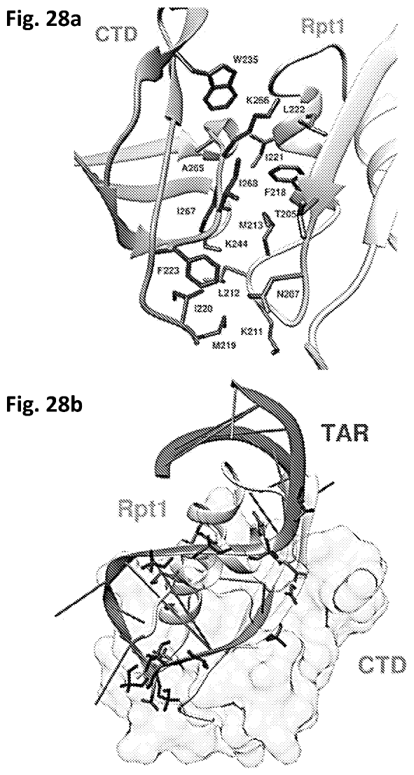

While some of the IN-CTD residues important for HIV-1 RNA interaction are known, the IN-CTD/TAR complex structure is unknown¹⁸. To determine the structural basis of similarity in binding of INI1 and TAR, we further refined _{IN-CTD/INI1183-304} complex model and also generated a second docking model for the IN-CTD/TAR interactions using IN-CTD [1QMC], this time by providing interaction restraints for the interface residues based on experimental data (see Methods), using the in-house docking software, MDockPP⁴³ (FIGS. 19a and b and Supplementary data 1). The docked complex with the lowest (best) score of ITScorePP^{44,45} indicated that upon complex formation ˜865.0 Å² of the solvent-accessible surface was buried (Table 4), which was larger than the buried surface area of the earlier IN-CTD/INI1183-304 model and the comparable SWIRM/INI1 Rpt1 complex (699.1 Å²,³⁵). This second modeled complex structure was in agreement with the earlier model (FIG. 14), where the negatively charged residues from α-1 helix of Rpt1 directly interacted with positively charged residues of IN-CTD. We could identify a total of 14 hydrogen bonds formed across the interface (FIG. 19b and FIG. 14). The 6 residues on IN-CTD (R228, W235, K244, R262, R263, and K264) and 6 residues on INI1-Rpt1 (E210, L212, M217, E220, D224, and D225) constituted a strip of hydrogen-bond network, establishing binding specificity (FIG. 19b). In addition, the binding affinity was conferred by hydrophobic interactions between INI1-Rpt1 and IN-CTD (FIG. 28a). On IN-CTD, a patch of the van der Waal surface that consists of a group of mostly hydrophobic residues (I220, F223, W235, A265, 1267, and 1268) matched the shape of a patch on Rpt1 that was also defined by mostly hydrophobic residues (L212, M213, F218, I221, and L222) (FIG. 28a). The region of the hydrophobic interactions was encircled by the residues forming the hydrogen-bonding network.

To understand the similarity in interaction of INI1-Rpt1 and TAR RNA with IN-CTD, we generated a docking model of IN-CTD/TAR RNA complex by using the lowest energy conformer from the NMR structures of TAR [1ANR] and that of IN-CTD [1QMC] and by using MDockPP⁴³. After applying the experimental restraints (see Methods), the top docked structure by reranking with ITScorePR⁴⁵ showed that the TAR RNA forms a complex with IN-CTD through the same interface region of IN-CTD as in the IN-CTD/INI1₁₈₃₋₃₀₄ complex, covering a similar net surface area of 826 Å² (FIG. 19c, d and Tables 4, 5 and Supplementary Data 1). This model indicated that the arginine and lysine residues of IN-CTD make multiple hydrogen bonds and electrostatic interactions with the phosphate backbone of TAR RNA (nucleotides 27-32 and 37-42) constituting the binding interface. IN-CTD specifically bound the minor groove of TAR RNA through 14 hydrogen bonds (similar to that of INI1-Rpt1) across the interface, including R228-U40, R228-U42, R262-C29, R263-C39, R269-U31, R269-G32, K244-C30, K266-A27, and K266-C39 (FIG. 19d). Meanwhile, the hydrophobic residues at the interface of IN-CTD protruded into the minor groove to form non-polar interaction with the hydrophobic part of the bases (FIG. 19d). The previous report indicated that nucleotide C30 of TAR and the loop were important for IN binding using CLIP-seq assay¹⁸. Furthermore, deletions, but not the nucleotide substitutions, of the loop and the adjacent three-nucleotide bulge of TAR reduced the IN binding, suggesting that IN prefers specific structural elements of TAR rather than specific nucleotide sequences¹⁸. Our model is consistent with this report and suggests that spatial positioning of the phosphate groups from the loop, the bulge, and the strand opposite of the loop in TAR RNA are important for interaction with IN residues.

When IN-CTD/TAR and IN-CTD/Rpt1 complexes were structurally aligned over IN-CTD, it became evident that both TAR and INI1-Rpt1 engage with the same binding interface and same residues of IN-CTD (FIG. 19e, f). There were only minor conformational differences in the side-chains in the IN-CTD interface residues, suggesting that no large side-chain rearrangements were required when switching from INI1-Rpt1 to TAR (FIG. 19e). The structural elements of INI1-Rpt1 and backbones of TAR seemed to perfectly line up in space (FIG. 19f). Moreover, the phosphate groups of TAR RNA were present in close proximity with the negatively charged residues of INI1-Rpt1 when the two docked complexes were superimposed (FIG. 28b). In addition, similar buried solvent accessible surface areas, 865 Å2 for IN-CTD/Rpt1 versus 826 Å² for IN-CTD/TAR, further supported that the two binding sites were the same for Rpt1 and TAR.

In the INI1-Rpt1 structure, the two β-sheets and helices of the INI1-Rpt1 (aa 183-248) come together to form a central hydrophobic barrel-like core, which is decorated by a string of negatively charged surface-exposed residues D192, E194, D196, E220, D224, D225 and D227 (FIG. 6a). Comparison of the arrangement of negatively charged residues of Rpt1 to the phosphate groups on the TAR RNA NMR structure (FIG. 6d), demonstrated a similar placement of negative charges on the two molecules. This observation, combined with the observation of the close proximity of the charged residues of INI1-Rpt1 with the phosphate residues of the TAR RNA when the two complexes were superimposed (FIG. 28b), suggest that the INI1-Rpt1 domain may structurally mimic TAR RNA involved in binding to IN-CTD (FIG. 6b, c).

As disclosed herein, we report a finding that suggests structural mimicry between INI1 Rpt1 domain and TAR RNA. This finding rests on a series of structural and functional studies. First, docking of NMR structures revealed that the IN-CTD interface residues required for INI1₁₈₃₋₂₆₅ binding overlap with those required for TAR RNA association. Second, mutational analyses validated the predicted structure of the INI1₁₈₃₋₂₆₅/IN-CTD complex. Third, binding experiments confirmed the requirement of the same IN-CTD residues for interaction with INI1₁₈₃₋₂₆₅ and TAR, indicating that Rpt1 and TAR recognize the same IN-CTD surface. Fourth, INI1₁₈₃₋₂₆₅ and TAR competed with each other for IN-CTD binding in vitro and in vivo. Finally, modeling the structure of IN-CTD/TAR complex suggested that the same positively charged residues of IN-CTD interacted with the negatively charged surface residues of INI1-Rpt1 and the phosphate groups of the TAR RNA. This was borne by the fact that when the NMR structures of the two molecules were compared, the distribution of negatively charged residues on INI1-Rpt1 were remarkably similar to the arrangement of the phosphate groups of interacting nucleotides on TAR. These studies together suggest that the Rpt1 domain of INI1 may be a TAR RNA mimic.

While Rpt1 (INI1₁₈₃₋₂₄₈) is a well-ordered structure, INI1 linker region (aa 249-265) is disordered. We propose that this region allows flexible positioning of Rpt1 relative to Rpt2 (aa 266-319), permitting Rpt1 to associate with various partners at different times, including IN and components of SWI/SNF. CryoEM studies of the intasome (the minimal unit for integration containing an IN tetramer with bound DNA) reveal that the IN-CTD domain exists in variable spatial positions in relation to CCD, due to the flexible linker region between CTD and CCD domains^{46,47}. It is likely that INI1-Rpt1 can interact with some of the CTD domains within the intasome based on their spatial positioning. We propose that the interaction between INI1-Rpt1 and IN-CTD remains as predicted by our model within the full length IN and INI1 complex, permitted by the flexible linker regions in the two proteins. A future cryoET analysis of the full length IN/INI1 complex or the IN/INI1/TAR RNA will be needed to reveal the higher order structures of these molecules.

Our model reveals extensive side-chain interactions at the IN-CTD/INI1₁₈₃₋₃₀₄ interface. D224 and D225 residues, part of the D224-D225-D227 Asp triad in the INI1-Rpt1 α1 helix, form hydrogen-bonding interactions with basic residues R228, K264, and R263 of IN-CTD. The W235 residue of IN-CTD exhibits hydrophobic interactions with INI1183-304. As predicted by our model, substituting W235 by charged residues W235E or W235K, but not by another aromatic side chain (W235F), disrupts IN/INI1 and the integration activity of IN in vivo³⁵. These studies not only validate the IN-CTD/_{INI1183-304} complex model but suggests that IN/INI1 binding may be important for integration.

While RNA mimicry by INI1 Rpt1 is unexpected, nucleic acid mimicry by proteins exists. Prokaryotic elongation factor-P (EF-P) mimics tRNA^{As}p and facilitates the elongation of difficult-to-synthesize proteins by alleviating ribosomal stalling during translation⁴⁸ and RRF (ribosomal recycling factor) and EF-G proteins mimic tRNA to regulate various stages of translation⁴⁹. Tumor suppressor p53 helix H2 mimics ssDNA and competes with it to bind to RPA (Replication protein A) 70 N complex to signal DNA damage⁵⁰. Moreover, Shq1p, an assembly factor for the biogenesis of ribosomes in yeast, mimics RNA, binds to the RNA-binding domain of Cbf5p and operates as a Cbf5p chaperone and an RNA placeholder during RNP assembly⁵¹.

What might be the role of TAR RNA mimicry by INI1-Rpt1 in HIV-1 replication?INI1 binds to IN within the context of GagPol and is incorporated into the virions in an IN-dependent manner^{10,11} (and current data). Since INI1-Rpt1 and TAR bind to the same IN surface, we propose that these two interactions with IN could be separated by space and time. We also propose that INI1 binding provides a “place-holder” function for RNA binding during assembly. Binding of INI1 to GagPol during assembly may prevent premature binding of RNA to IN to prevent a possible steric hindrance (FIG. 6e, panel 1). Since INI1 and TAR RNA can compete with each other and bind to the same surface of IN, INI1 may compete off RNA binding to IN within GagPol. Class II-mutants defective for binding to RNA or INI1-interaction-defective-IN mutants within GagPol would be expected to fail to bind RNA, relieving the aforementioned steric hindrance permitting assembly (FIG. 6e, panel 2). Thus, INI1 may act as a “place-holder”, which would be critical for assembly and particle production. Indeed, lack of INI1 inhibits assembly and particle production^{6,9,11} (FIG. 6e, panel 3). Once assembly is completed and upon Gag proteolysis, INI1 binding may be displaced by RNA binding to IN during particle maturation. Since INI1 and RNA binding surfaces on IN overlap, it is not possible to distinguish between the roles of these two interacting partners during particle maturation within the virions at this point.

It is conceivable that the suggested mimicry between TAR and INI1-Rpt1 domain may have first evolved to modulate HIV-1 transcription, and the effects on assembly are rather consequential. INI1 directly binds to acetylated Tat¹⁹, and together with the SWI/SNF complex, it facilitates Tat-mediated transcriptional elongation^{12,19,52}. It is possible that the suggested TAR mimicry of INI1-Rpt1 domain allows it bind to Tat, which may be important for recruiting SWI/SNF complex to sites of LTR transcription to facilitate elongation⁵². Future experiments on TAR RNA mimicry of INI1 is likely to lead to a better understanding of Tat-INI1 and IN-INI1 interactions and may lead to the development of a unique class of drugs to inhibit multiple stages of HIV-1 replication.

Since INI1-Rpt1 domain is highly conserved among eukaryotes and HIV-1 is of recent origin, it is likely that Rpt1 domain has evolved to mimic a cellular RNA, rather than HIV-1 RNA. TAR RNA mimics cellular 7SK RNA at the SL1 (stem loop 1)⁵³. Therefore, it is plausible that INI1-Rpt1 may have evolved to mimic 7SK. Thus, future experiments to understand the possible INI1-Rpt1 RNA mimicry are likely to unravel insights about INI1 role not only in HIV-1 replication, but also in cellular transcription and tumor suppressor function.

### REFERENCES FOR EXAMPLES 7-12

1. Savas, S. & Skardasi, G. The SWI/SNF complex subunit genes: their functions, variations, and links to risk and survival outcomes in human cancers. Crit. Rev. Oncol./Hematol. 123, 114-131 (2018).
2. Kadoch, C. & Crabtree, G. R. Mammalian SWI/SNF chromatin remodeling complexes and cancer: mechanistic insights gained from human genomics. Sci. Adv. 1, e1500447 (2015).
3. Kalpana, G. V., Marmon, S., Wang, W., Crabtree, G. R. & Goff, S. P. Binding and stimulation of HIV-1 integrase by a human homolog of yeast transcription factor SNF5 [see comments]. Science 266, 2002-2006 (1994).
4. Cano, J. & Kalpana, G. V. Inhibition of early stages of HIV-1 assembly by INI1/hSNF5 transdominant negative mutant S6. J. Virol. 85, 2254-2265 (2011).
5. Das, S., Cano, J. & Kalpana, G. V. Multimerization and DNA binding properties of INI1/hSNF5 and its functional significance. J. Biol. Chem. 284, 19903-19914 (2009).
6. La Porte, A., Cano, J., Wu, X., Mitra, D. & Kalpana, G. V. An essential role of INI1/hSNF5 chromatin remodeling protein in HIV-1 posttranscriptional events and Gag/Gag-pol stability. J. Virol. 90, 9889-9904 (2016).
7. Mathew, S. et al. INI1/hSNF5-interaction defective HIV-1 IN mutants exhibit impaired particle morphology, reverse transcription and integration in vivo. Retrovirology 10, 66 (2013).
8. Sorin, M. et al. Recruitment of a SAP18-HDAC1 complex into HIV-1 vinons and its requirement for viral replication. PLoS Pathog. 5, e1000463 (2009).
9. Sorin, M., Yung, E., Wu, X. & Kalpana, G. V. HIV-1 replication in cell lines harboring INI1/hSNF5 mutations. Retrovirology 3, 56 (2006).
10. Yung, E. et al. Inhibition of HIV-1 virion production by a transdominant mutant of integrase interactor 1. Nat. Med. 7, 920-926 (2001).
11. Yung, E. et al. Specificity of interaction of INI1/hSNF5 with retroviral integrases and its functional significance. J. Virol. 78, 2222-2231 (2004).
12. Mahmoudi, T. et al. The SWI/SNF chromatin-remodeling complex is a cofactor for Tat transactivation of the HIV promoter. J. Biol. Chem. 281, 19960-19968 (2006).
13. Lesbats, P. et al. Functional coupling between HIV-1 integrase and the SWI/SNF chromatin remodeling complex for efficient in vitro integration into stable nucleosomes. PLoS Pathog. 7, e1001280 (2011).
14. Parissi, V. et al. Inactivation of the SNF5 transcription factor gene abolishes the lethal phenotype induced by the expression of HIV-1 integrase in yeast. Gene 247, 129-136 (2000).
15. Elliott, J. L. et al. Integrase-RNA interactions underscore the critical role of integrase in HIV-1 virion morphogenesis. eLife 9, https://doi.org/10.7554/eLife.54311 (2020).
16. Elliott, J. L. & Kutluay, S. B. Going beyond Integration: The Emerging Role of HIV-1 Integrase in Virion Morphogenesis. Viruses 12, https://doi.org/10.3390/v12091005 (2020).
17. Engelman, A. N. Multifaceted HIV integrase functionalities and therapeutic strategies for their inhibition. J. Biol. Chem. 294, 15137-15157 (2019).
18. Kessl, J. J. et al. HIV-1 integrase binds the viral RNA genome and is essential during virion morphogenesis. Cell 166, 1257-1268 e1212 (2016).
19. Ariumi, Y., Serhan, F., Turelli, P., Telenti, A. & Trono, D. The integrase interactor 1 (INI1) proteins facilitate Tat-mediated human immunodeficiency virus type 1 transcription. Retrovirology 3, 47 (2006). 1742-4690-3-47 [pii].
20. Cheng, S.-W. G. et al. c-MYC interacts with INI1/hSNF5 and requires the SWI/SNF complex for transactivation function. Nat. Genet. 22, 102-105 (1999).
21. Hwang, S., Lee, D., Gwack, Y., Min, H. & Choe, J. Kaposi's sarcoma-associated herpesvirus K8 protein interacts with hSNF5. J. Gen. Virol. 84, 665-676 (2003).
22. Lee, D., Sohn, H., Kalpana, G. V. & Choe, J. Interaction of El and hSNF5 proteins stimulates replication of human papillomavirus DNA. Nature 399, 487-491 (1999).
23. Wu, D. Y., Kalpana, G. V., Goff, S. P. & Schubach, W. H. Epstein-Barr virus nuclear protein 2 (EBNA2) binds to a component of the human SNF-SWI complex, hSNF5/Inil. J. Virol. 70, 6020-6028 (1996).
24. Morozov, A., Yung, E. & Kalpana, G. Structure-function analysis of integrase interactor 1/hSNF5L1 reveals differential properties of two repeat motifs present in the highly conserved region. Proc. Natl Acad. Sci. USA 95, 1120-1125 (1998).
25. Kim, D. E., Chivian, D. & Baker, D. Protein structure prediction and analysis using the Robetta server. Nucleic Acids Res. 32, W526-W531 (2004).
26. Laskowski, R. A., Rullmannn, J. A., MacArthur, M. W., Kaptein, R. & Thornton, J. M. AQUA and PROCHECK-NMR: programs for checking the quality of protein structures solved by NMR. J. Biomol. NMR 8, 477-486 (1996).
27. Vriend, G. WHAT IF: a molecular modeling and drug design program. J. Mol. Graph 8, 52-56 (1990). 29.
28. Eisenberg, D., Luthy, R. & Bowie, J. U. VERIFY3D: assessment of protein models with three-dimensional profiles. Methods Enzymol. 277, 396-404 (1997).
29. Wiederstein, M. & Sippl, M. J. ProSA-web: interactive web service for the recognition of errors in three-dimensional structures of proteins. Nucleic Acids Res. 35, W407-W410 (2007).
30. He, S. et al. Structure of nucleosome-bound human BAF complex. Science 367, 875-881 (2020).
31. Eijkelenboom, A. P. et al. Refined solution structure of the C-terminal DNA-binding domain of human immunovirus-1 integrase. Proteins 36, 556-564 (1999).
32. Eijkelenboom, A. P. A. M. et al. The DNA-binding domain of HIV-1 integrase has an SH3-like fold. Nat. Struct. Biol. 2, 807-810 (1995).
33. Yan, L., Xie, S., Du, Y. & Qian, C. Structural Insights into BAF47 and BAF155 complex formation. J. Mol. Biol. 429, 1650-1660 (2017).
34. Cannon, P. M., Byles, E. D., Kingsman, S. M. & Kingsman, A. J. Conserved sequences in the carboxyl terminus of integrase that are essential for human immunodeficiency virus type 1 replication. J. Virol. 70, 651-657 (1996).
35. Li, M. & Craigie, R. Processing of viral DNA ends channels the HIV-1 integration reaction to concerted integration. J. Biol. Chem. 280, 29334-29339 (2005).
36. Engelman, A. In vivo analysis of retroviral integrase structure and function. Adv. Virus Res. 52, 411-426 (1999).
37. Yasgar, A., Jadhav, A., Simeonov, A. & Coussens, N. P. Alphascreen-based assays: ultra-high-throughput screening for small-molecule inhibitors of challenging enzymes and protein-protein interactions. Methods Mol. Biol. 1439, 77-98 (2016).
38. Cassel, J. A., Blass, B. E., Reitz, A. B. & Pawlyk, A. C. Development of a novel nonradiometric assay for nucleic acid binding to TDP-43 suitable for high-throughput screening using AlphaScreen technology. J. Biomol. Screen 15, 1099-1106 (2010).
39. Lu, R., Limon, A., Ghory, H. Z. & Engelman, A. Genetic analyses of DNA-binding mutants in the catalytic core domain of human immunodeficiency virus type 1 integrase. J. Virol. 79, 2493-2505 (2005). 79/4/2493 [pii].
40. Fontana, J. et al. Distribution and redistribution of HIV-1 nucleocapsid protein in immature, mature, and integrase-inhibited virions: a role for integrase in maturation. J. Virol. 89, 9765-9780 (2015).
41. Jurado, K. A. et al. Allosteric integrase inhibitor potency is determined through the inhibition of HIV-1 particle maturation. Proc. Natl Acad. Sci. USA 110, 8690-8695 (2013).
42. Leavitt, A. D., Robles, G., Alesandro, N. & Varmus, H. E. Human immunodeficiency virus type 1 integrase mutants retain in vitro integrase activity yet fail to integrate viral DNA efficiently during infection. J. Virol. 70, 721-728 (1996).
43. Xu, X. et al. Performance of MDockPP in CAPRI rounds 28-29 and 31-35 including the prediction of water-mediated interactions. Proteins 85, 424-434 (2017).
44. Huang, S. Y. et al. Inclusion of the orientational entropic effect and low-resolution experimental information for protein-protein docking in Critical Assessment of PRedicted Interactions (CAPRI). Proteins 81, 2183-2191 (2013).
45. Huang, S. Y. & Zou, X. A knowledge-based scoring function for protein-RNA interactions derived from a statistical mechanics-based iterative method. Nucleic Acids Res. 42, e55 (2014).
46. Ballandras-Colas, A. et al. Cryo-EM reveals a novel octameric integrase structure for betaretroviral intasome function. Nature 530, 358-361 (2016).
47. Passos, D. O. et al. Cryo-EM structures and atomic model of the HIV-1 strand transfer complex intasome. Science 355, 89-92 (2017).
48. Katz, A., Solden, L., Zou, S. B., Navarre, W. W. & Ibba, M. Molecular evolution of protein-RNA mimicry as a mechanism for translational control. Nucleic Acids Res. 42, 3261-3271 (2014).
49. Tsonis, P. A. & Dwivedi, B. Molecular mimicry: structural camouflage of proteins and nucleic acids. Biochim. Biophys. Acta 1783, 177-187 (2008).
50. Bochkareva, E. et al. Single-stranded DNA mimicry in the p53 transactivation domain interaction with replication protein A. Proc. Natl Acad. Sci. USA 102, 15412-15417 (2005).
51. Walbott, H. et al. The H/ACA RNP assembly factor SHQ1 functions as an RNA mimic. Genes Dev. 25, 2398-2408 (2011).
52. Treand, C. et al. Requirement for SWI/SNF chromatin-remodeling complex in Tat-mediated activation of the HIV-1 promoter. EMBO J. 25, 1690-1699 (2006). 7601074 [pii].
53. Pham, V. V. et al. HIV-1 Tat interactions with cellular 7SK and viral TAR RNAs identifies dual structural mimicry. Nat. Commun. 9, 4266 (2018).
54. Li, M. Z. & Elledge, S. J. Harnessing homologous recombination in vitro to generate recombinant DNA via SLIC. Nat. Methods 4, 251-256 (2007).
55. Kazimierczuk, K. & Orekhov, V. Y. Accelerated NMR spectroscopy by using compressed sensing. Angew. Chem. Int Ed. Engl. 50, 5556-5559 (2011).
56. Delaglio, F. et al. NMRPipe: a multidimensional spectral processing system based on UNIX pipes. J. Biomol. NMR 6, 277-293 (1995).
57. Skinner, S. P. et al. CcpNmr AnalysisAssign: a flexible platform for integrated NMR analysis. J. Biomol. NMR 66, 111-124 (2016).
58. Philo, J. S. A method for directly fitting the time derivative of sedimentation velocity data and an alternative algorithm for calculating sedimentation coefficient distribution functions. Anal. Biochem. 279, 151-163 (2000).
59. Philo, J. S. Improved methods for fitting sedimentation coefficient distributions derived by time-derivative techniques. Anal. Biochem. 354, 238-246 (2006).
60. Ye, Z. et al. WHATIF: An open-source desktop application for extraction and management of the incidental findings from next-generation sequencing variant data. Comput Biol. Med. 68, 165-169 (2016).
61. Colovos, C. & Yeates, T. O. Verification of protein structures: patterns of nonbonded atomic interactions. Protein Sci. 2, 1511-1519 (1993).
62. van Zundert, G. C. P. et al. The HADDOCK2.2 web server: user-friendly integrative modeling of biomolecular complexes. J. Mol. Biol. 428, 720-725 (2016).
63. Pettersen, E. F. et al. UCSF Chimera-a visualization system for exploratory research and analysis. J. Comput. Chem. 25, 1605-1612 (2004).
64. Welker, R., Hohenberg, H., Tessmer, U., Huckhagel, C. & Krausslich, H. G. Biochemical and structural analysis of isolated mature cores of human immunodeficiency virus type 1. J. Virol. 74, 1168-1177 (2000).
65. Butler, S. L., Hansen, M. S. & Bushman, F. D. A quantitative assay for HIV DNA integration in vivo. Nat. Med. 7, 631-634 (2001).
66. Liszewski, M. K., Yu, J. J. & O'Doherty, U. Detecting HIV-1 integration by repetitive-sampling Alu-gag PCR. Methods 47, 254-260 (2009). S1046-2023 (09)00003-6 [pii].
67. Fontana, J., Cardone, G., Heymann, J. B., Winkler, D. C. & Steven, A. C. Structural changes in Influenza virus at low pH characterized by cryo-electron tomography. J. Virol. 86, 2919-2929 (2012).
68. Mastronarde, D. N. Automated electron microscope tomography using robust prediction of specimen movements. J. Struct. Biol. 152, 36-51 (2005).
69. Heymann, J. B., Cardone, G., Winkler, D. C. & Steven, A. C. Computational resources for cryo-electron tomography in Bsoft. J. Struct. Biol. 161, 232-242 (2008).
70. Frangakis, A. S. & Hegerl, R. Noise reduction in electron tomographic reconstructions using nonlinear anisotropic diffusion. J. Struct. Biol. 135, 239-250 (2001).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

1. A composition, comprising
a peptide having at least about 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 1 (lys-Leu-Met-Thr-Pro-Glu-Met-Phe-Ser-Glu-Ile-Leu-Cys-Asp-Leu-Asn); and/or
a peptide having at least about 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO. 1 (lys-Leu-Met-Thr-Pro-Glu-Met-Phe-Ser-Glu-Ile-Leu-Cys-Asp-Leu-Asn) and one or more unnatural amino acids,
or a pharmaceutically acceptable salt, a metabolite, or a carrier thereof.

2. The composition according to claim 1, wherein the peptide is stapled by forming a covalent linkage between the side-chains of the one or more unnatural amino acids.

3. The composition according to claim 1 or 2, wherein the one or more unnatural amino acids comprises (S)-2-(4-pentenyl) alanine and/or (R)-2-(7-octenyl) alanine.

4. The composition according to claim 1, wherein two unnatural amino acids are present within the peptide, and wherein the two unnatural amino acids are located within about 1, 2, 3, 4, 5, 6, 7, 8, 9, 10, 11, 13, 14, or 15 amino acid apart from each other.

5. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-X-Glu-Ile-Leu-X-Cys-Asp-Leu-Asn), wherein X is a non-natural amino acid.

6. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 2 (lys-Leu-Met-Thr-Pro-Glu-Met-Phe-S5-Glu-Ile-Leu-S5-Cys-Asp-Leu-Asn), wherein S5 is (S)-2-(4-pentenyl) alanine.

7. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-S5-Glu-Ile-Leu-R8-Cys-Asp-Leu-Asn), wherein R8 is (R)-2-(7-octenyl) alanine and S5 is (S)-2-(4-pentenyl) alanine.

8. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-R8-Glu-Ile-Leu-S5-Cys-Asp-Leu-Asn), wherein R8 is (R)-2-(7-octenyl) alanine and S5 is (S)-2-(4-pentenyl) alanine.

9. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-X-Met-Phe-Glu-Ile-Leu-X-Cys-Asp-Leu-Asn), wherein X is a non-natural amino acid.

10. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 3 (lys-Leu-Met-Thr-Pro-Glu-R8-Met-Phe-Glu-Ile-Leu-S5-Cys-Asp-Leu-Asn), wherein R8 is (R)-2-(7-octenyl) alanine and S5 is (S)-2-(4-pentenyl) alanine.

11. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 3 (lys-Leu-Met-Thr-Pro-Glu-S5-Met-Phe-Glu-Ile-Leu-R8-Cys-Asp-Leu-Asn), wherein R8 is (R)-2-(7-octenyl) alanine and S5 is (S)-2-(4-pentenyl) alanine.

12. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 3 (lys-Leu-Met-Thr-Pro-Glu-S5-Met-Phe-Glu-Ile-Leu-S5-Cys-Asp-Leu-Asn), wherein S5 is (S)-2-(4-pentenyl) alanine.

13. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-X-Glu-Ile-Leu-X-Cys-Gly-Leu-Asn), wherein X is a non-natural amino acid.

14. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to SEQ ID NO: 4 (lys-Leu-Met-Thr-Pro-Glu-Met-Phe-S5-Glu-Ile-Leu-S5-Cys-Gly-Leu-Asn), wherein S5 is (S)-2-(4-pentenyl) alanine.

15. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-S5-Glu-Ile-Leu-R8-Cys-Gly-Leu-Asn), wherein R8 is (R)-2-(7-octenyl) alanine and S5 is (S)-2-(4-pentenyl) alanine.

16. The composition according to claim 1, wherein the peptide is stapled and has at least 80%, 81%, 82%, 83%, 84%, 85%, 86%, 87%, 88%, 89%, 90%, 91%, 92%, 93%, 94%, 95%, 96%, 97%, 98%, 99%, and/or 100% sequence identity to (lys-Leu-Met-Thr-Pro-Glu-Met-Phe-R8-Glu-Ile-Leu-S5-Cys-Gly-Leu-Asn), wherein R8 is (R)-2-(7-octenyl) alanine and S5 is (S)-2-(4-pentenyl) alanine.

17. (canceled)

18. (canceled)

19. The composition according to claim 1, further comprising at least one antiretroviral agent and/or at least one latency reversing agent (LRA).

20. (canceled)

21. (canceled)

22. (canceled)

23. A method of treating an HIV infection, comprising:
administering to a subject an agent that inhibits or disrupts (i) the interaction between IN and INI1/SMARCB1 complex and/or (ii) the interaction between IN and TAR RNA.

24. The method of claim 23, wherein the subject is administered with the composition according to claim 1.

25. (canceled)

26. The method according to claim 23, wherein the HIV infection further comprises (i) a condition and/or a disease associated with an HIV-infection, (ii) acquired immunodeficiency syndrome (AIDS), or (iii) a combination thereof.

27. (canceled)

28. A method of reducing a side effect of a therapeutic regime, comprising:
administering to a subject the composition according to claim 1, wherein: the subject has received at least one therapeutic regime selected from surgery, antiretroviral therapy (ART), highly active antiretroviral therapy (HAART) or a combination thereof, and the subject is experiencing at least one side effect as a consequence of the therapeutic regime.

29. (canceled)

30. (canceled)

31. (canceled)

32. (canceled)


SEQUENCE LISTING (61 sequences)

SEQ ID NO: 1 - protein, 16 aa, Artificial Sequence
  Description of Artificial Sequence Synthetic peptide
KLMTPEMFSEILCDLN

SEQ ID NO: 2 - protein, 17 aa, Artificial Sequence
  Description of Artificial Sequence Synthetic peptide
  MOD_RES (9)..(9): (S)-2-(4-pentenyl) alanine
  MOD_RES (13)..(13): (S)-2-(4-pentenyl) alanine
KLMTPEMFAEILACDLN

SEQ ID NO: 3 - protein, 17 aa, Artificial Sequence
  Description of Artificial Sequence Synthetic peptide
  MOD_RES (7)..(7): (R)-2-(7-octenyl) alanine
  MOD_RES (13)..(13): (S)-2-(4-pentenyl) alanine
KLMTPEAMFEILACDLN

SEQ ID NO: 4 - protein, 17 aa, Artificial Sequence
  Description of Artificial Sequence Synthetic peptide
  MOD_RES (9)..(9): (S)-2-(4-pentenyl) alanine
  MOD_RES (13)..(13): (S)-2-(4-pentenyl) alanine
KLMTPEMFAEILACGLN

SEQ ID NO: 5 - DNA, 26 bp, Artificial Sequence
  Description of Artificial Sequence Synthetic primer
tccgaattcgagctccgtcgacaagc

SEQ ID NO: 6 - DNA, 27 bp, Artificial Sequence
  Description of Artificial Sequence Synthetic primer
tccaccaatctgttctctgtgagcctc

SEQ ID NO: 7 - DNA, 55 bp, Artificial Sequence
  Description of Artificial Sequence Synthetic primer
ggctcacagagaacagattggtggatcccccgaggtgctggtccccatccggctg

SEQ ID NO: 8 - DNA, 56 bp, Artificial Sequence
  Description of Artificial Sequence Synthetic primer
gcttgtcgacggagctcgaattcggattactacttgatgatgacgcgctggtctga

SEQ ID NO: 9 - DNA, 59 bp, Artificial Sequence
  Description of Artificial Sequence Synthetic primer
gcttgtcgacggagctcgaattcggattagcccaaccccagctccgagcacagcttcag

SEQ ID NO: 10 - DNA, 35 bp, Artificial Sequence
  Description of Artificial Sequence Synthetic primer
gcgggttcaaatccagaccgtcacagaggatttct

SEQ ID NO: 11 - DNA, 35 bp, Artificial Sequence
  Description of Artificial Sequence Synthetic primer
agaaatcctctgtgacggtctggatttgaacccgc

SEQ ID NO: 12 - DNA, 39 bp, Artificial Sequence
  Description of Artificial Sequence Synthetic primer
catgaatgagaagttgatggcgcctgagatgttttcaga

SEQ ID NO: 13 - DNA, 39 bp, Artificial Sequence
  Description of Artificial Sequence Synthetic primer
tctgaaaacatctcaggagccatcaacttctcattcatg

SEQ ID NO: 14 - DNA, 33 bp, Artificial Sequence
  Description of Artificial Sequence Synthetic primer
cgtcagcgggttcaaacccagatcgtcacagag

SEQ ID NO: 15 - DNA, 33 bp, Artificial Sequence
  Description of Artificial Sequence Synthetic primer
ctctgtgacgatctgggtttgaacccgctgacg

SEQ ID NO: 16 - DNA, 35 bp, Artificial Sequence
  Description of Artificial Sequence Synthetic primer
gaaatcctctgtgacgagctggatttgaacccgct

SEQ ID NO: 17 - DNA, 35 bp, Artificial Sequence
  Description of Artificial Sequence Synthetic primer
agcgggttcaaatccagctcgtcacagaggatttc

SEQ ID NO: 18 - DNA, 41 bp, Artificial Sequence
  Description of Artificial Sequence Synthetic primer
tttgctggtcctttgaaaagtggatttctgctgtccctgta

SEQ ID NO: 19 - DNA, 41 bp, Artificial Sequence
  Description of Artificial Sequence Synthetic primer
tacagggacagcagaaatccacttttcaaaggaccagcaaa

SEQ ID NO: 20 - DNA, 40 bp, Artificial Sequence
  Description of Artificial Sequence Synthetic primer
gacctttgctggtcctttctcaagtggatttctgctgctc

SEQ ID NO: 21 - DNA, 40 bp, Artificial Sequence
  Description of Artificial Sequence Synthetic primer
ggacagcagaaatccacttgagaaaggaccagcaaagctc

SEQ ID NO: 22 - DNA, 40 bp, Artificial Sequence
  Description of Artificial Sequence Synthetic primer
gagctttgctggtcctttcgcaagtggatttctgctgctc

SEQ ID NO: 23 - DNA, 40 bp, Artificial Sequence
  Description of Artificial Sequence Synthetic primer
ggacagcagaaatccacttgcgaaaggaccagcaaagctc

SEQ ID NO: 24 - DNA, 40 bp, Artificial Sequence
  Description of Artificial Sequence Synthetic primer
ggacagcagaaatccacttaagaaaggaccagcaaagctc

SEQ ID NO: 25 - DNA, 40 bp, Artificial Sequence
  Description of Artificial Sequence Synthetic primer
gagctttgctggtcctttcttaagtggatttctgctgctc

SEQ ID NO: 26 - DNA, 66 bp, Artificial Sequence
  Description of Artificial Sequence Synthetic primer
ccatctgttttccataatccctaatgatcgctgctgctcttcttggcactacttttatgtcactat

SEQ ID NO: 27 - DNA, 66 bp, Artificial Sequence
  Description of Artificial Sequence Synthetic primer
atagtgacataaaagtagtgccaagaagagcagcagcgatcattagggattatggaaaacagatgg

SEQ ID NO: 28 - DNA, 55 bp, Artificial Sequence
  Description of Artificial Sequence Synthetic primer
cacctgccatctgtgctccataatccgcaatgatctttgcttttcttcttggcac

SEQ ID NO: 29 - DNA, 55 bp, Artificial Sequence
  Description of Artificial Sequence Synthetic primer
gtgccaagaagaaaagcaaagatcattgcggattatggagcacagatggcaggtg

SEQ ID NO: 30 - DNA, 52 bp, Artificial Sequence
  Description of Artificial Sequence Synthetic primer
caaagtggatttctgctgtccgcgtaataaacccgaaaattttgaatttttg

SEQ ID NO: 31 - DNA, 52 bp, Artificial Sequence
  Description of Artificial Sequence Synthetic primer
caaaaattcaaaattttcgggtttattacgcggacagcagaaatccactttg

SEQ ID NO: 32 - DNA, 36 bp, Artificial Sequence
  Description of Artificial Sequence Synthetic primer
ctactgccccttcacctgcccagaggagctttgctg

SEQ ID NO: 33 - DNA, 36 bp, Artificial Sequence
  Description of Artificial Sequence Synthetic primer
cagcaaagctcctctgggcaggtgaaggggcagtag

SEQ ID NO: 34 - DNA, 59 bp, Artificial Sequence
  Description of Artificial Sequence Synthetic oligonucleotide
  Description of Combined DNA/RNA Molecule Synthetic oligonucleotide
  modified_base (1)..(1): Biotin deoxythymidine
  modified_base (59)..(59): Biotin deoxythymidine
tggucucucugguuagaccagaucugagccugggagcucucuggcuaacuagggaacct

SEQ ID NO: 35 - RNA, 57 nt, Human immunodeficiency virus 1
ggucucucugguuagaccagaucugagccugggagcucucuggcuaacuagggaacc

SEQ ID NO: 36 - RNA, 43 nt, Human immunodeficiency virus 1
gcaggacucggcuugcugaagcgcgcacggcaagaggcgaggg

SEQ ID NO: 37 - DNA, 20 bp, Artificial Sequence
  Description of Artificial Sequence Synthetic primer
gtgcccgtctgttgtgtgac

SEQ ID NO: 38 - DNA, 20 bp, Artificial Sequence
  Description of Artificial Sequence Synthetic primer
ggcgccactgctagagattt

SEQ ID NO: 39 - DNA, 34 bp, Artificial Sequence
  Description of Artificial Sequence Synthetic probe
ctagagatccctcagacccttttagtcagtgtgg

SEQ ID NO: 40 - DNA, 20 bp, Artificial Sequence
  Description of Artificial Sequence Synthetic primer
tgtgtgcccgtctgttgtgt

SEQ ID NO: 41 - DNA, 20 bp, Artificial Sequence
  Description of Artificial Sequence Synthetic primer
gagtcctgcgtcgagagagc

SEQ ID NO: 42 - DNA, 20 bp, Artificial Sequence
  Description of Artificial Sequence Synthetic probe
cagtggcgcccgaacaggga

SEQ ID NO: 43 - DNA, 23 bp, Artificial Sequence
  Description of Artificial Sequence Synthetic primer
aactagggaacccactgcttaag

SEQ ID NO: 44 - DNA, 24 bp, Artificial Sequence
  Description of Artificial Sequence Synthetic primer
tccacagatcaaggatatcttgtc

SEQ ID NO: 45 - DNA, 30 bp, Artificial Sequence
  Description of Artificial Sequence Synthetic probe
acactacttgaagcactcaaggcaagcttt

SEQ ID NO: 46 - DNA, 25 bp, Artificial Sequence
  Description of Artificial Sequence Synthetic primer
gcctcccaaagtgctgggattacag

SEQ ID NO: 47 - DNA, 20 bp, Artificial Sequence
  Description of Artificial Sequence Synthetic primer
gttcctgctatgtcacttcc

SEQ ID NO: 48 - protein, 17 aa, Artificial Sequence
  Description of Artificial Sequence Synthetic peptide
  MOD_RES (9)..(9): Any non-natural amino acid
  MOD_RES (13)..(13): Any non-natural amino acid
KLMTPEMFXEILXCDLN

SEQ ID NO: 49 - protein, 17 aa, Artificial Sequence
  Description of Artificial Sequence Synthetic peptide
  MOD_RES (9)..(9): (S)-2-(4-pentenyl) alanine
  MOD_RES (13)..(13): (R)-2-(7-octenyl) alanine
KLMTPEMFAEILACDLN

SEQ ID NO: 50 - protein, 17 aa, Artificial Sequence
  Description of Artificial Sequence Synthetic peptide
  MOD_RES (9)..(9): (R)-2-(7-octenyl) alanine
  MOD_RES (13)..(13): (S)-2-(4-pentenyl) alanine
KLMTPEMFAEILACDLN

SEQ ID NO: 51 - protein, 17 aa, Artificial Sequence
  Description of Artificial Sequence Synthetic peptide
  MOD_RES (7)..(7): Any non-natural amino acid
  MOD_RES (13)..(13): Any non-natural amino acid
KLMTPEXMFEILXCDLN

SEQ ID NO: 52 - protein, 17 aa, Artificial Sequence
  Description of Artificial Sequence Synthetic peptide
  MOD_RES (7)..(7): (S)-2-(4-pentenyl) alanine
  MOD_RES (13)..(13): (R)-2-(7-octenyl) alanine
KLMTPEAMFEILACDLN

SEQ ID NO: 53 - protein, 17 aa, Artificial Sequence
  Description of Artificial Sequence Synthetic peptide
  MOD_RES (7)..(7): (S)-2-(4-pentenyl) alanine
  MOD_RES (13)..(13): (S)-2-(4-pentenyl) alanine
KLMTPEAMFEILACDLN

SEQ ID NO: 54 - protein, 17 aa, Artificial Sequence
  Description of Artificial Sequence Synthetic peptide
  MOD_RES (9)..(9): Any non-natural amino acid
  MOD_RES (13)..(13): Any non-natural amino acid
KLMTPEMFXEILXCGLN

SEQ ID NO: 55 - protein, 17 aa, Artificial Sequence
  Description of Artificial Sequence Synthetic peptide
  MOD_RES (9)..(9): (S)-2-(4-pentenyl) alanine
  MOD_RES (13)..(13): (R)-2-(7-octenyl) alanine
KLMTPEMFAEILACGLN

SEQ ID NO: 56 - protein, 17 aa, Artificial Sequence
  Description of Artificial Sequence Synthetic peptide
  MOD_RES (9)..(9): (R)-2-(7-octenyl) alanine
  MOD_RES (13)..(13): (S)-2-(4-pentenyl) alanine
KLMTPEMFAEILACGLN

SEQ ID NO: 57 - protein, 18 aa, Human immunodeficiency virus 1
MTPEMSFEILCDDLDLNP

SEQ ID NO: 58 - protein, 6 aa, Artificial Sequence
  Description of Artificial Sequence Synthetic 6xHis tag
HHHHHH

SEQ ID NO: 59 - protein, 18 aa, Artificial Sequence
  Description of Artificial Sequence Synthetic peptide
MTPEMSFEILCDGLDLNP

SEQ ID NO: 60 - protein, 18 aa, Artificial Sequence
  Description of Artificial Sequence Synthetic peptide
MAPEMSFEILCDDLDLNP

SEQ ID NO: 61 - protein, 18 aa, Artificial Sequence
  Description of Artificial Sequence Synthetic peptide
MTPEMSFEILCDDLGLNP